(19) United States
(12) Patent Application Publication
(10) Pub. No.: US 2023/0226203 A1
(43) Pub. Date: Jul. 20, 2023

(54) ACTIVATABLE PROCYTOKINES

(71) Applicant: Proviva Therapeutics (Hong Kong) Limited, Hong kong (CN)

(72) Inventors: Zijuan LI, Shanghai (CN); Hongxing ZHOU, Bedford, MA (US)

(21) Appl. No.: 18/010,081

(22) PCT Filed: Jun. 18, 2020
(86) PCT No.: PCT/CN2020/096903

§ 371 Date: Dec. 13, 2022

(51) Int. Cl. A61K 47/64; A61K 47/65

(52) U.S. Cl. CPC A61K 47/6425; A61K 47/642; A61K 47/65

(57) ABSTRACT

Provided are activatable proprotein or procytokine homodimers composed of at least two separate polypeptide chains, each chain comprising a binding moiety such as an Fc region, a cytokine, a cytokine receptor, and at least one a cleavable linker, among other optional features, and related pharmaceutical compositions and methods of use thereof.

### BACKGROUND

### Technical Field

The present disclosure relates to activatable proprotein or procytokine homodimers composed of at least two separate polypeptide chains, each chain comprising a binding moiety such as an Fc region, a cytokine, a cytokine receptor, and at least one a cleavable linker, among other optional features, and related pharmaceutical compositions and methods of use thereof.

### Description of the Related Art

Cytokine and other ligand therapies have utility in a variety of disease indications. For instance, interleukin-2 (IL-2), interleukin-7 (IL-7), interleukin-15 (IL-15), interleukin-21 (IL-21), and interferon immunotherapies have proven or potential utility in the treatment of various diseases or conditions, including cancers such as malignant melanoma and renal cell cancer, chronic infections such as HIV infections, inflammatory diseases, and transplant therapies.

However, there are certain problems associated with most cytokine therapies, such as IL-2 and IL-15 therapies. For example, current forms of IL-2 therapy have a short half-life in circulation and predominantly expand immunosuppressive regulatory T cells, or T_{regs} (see, for example, Arenas-Ramirez et al., Trends in Immunology. 36: 763-777, 2015). Moreover, the effects of IL-2 therapy are predominantly systemic, rather than being localized to target tissues, resulting in many severe side effects such as breathing problems, nausea, low blood pressure, loss of appetite, confusion, serious infections, seizures, allergic reactions, heart problems, renal failure, and vascular leak syndrome.

Similarly, IL-15 has been shown to exhibit a short half-life and high doses can be required to achieve biological responses in vivo, resulting in clinical toxicities and limited anti-tumor responses in patients. IL-15 and IL-15 derivatives are under development to increase therapeutic effectiveness. However, significant drawbacks exist, including high serum Cmax initially causing over-activation of immune system, short PK due to either small molecular size for IL-15 (13-14 kD) or catabolism by the large number of immune cells expressing IL-15 receptors for IL-15 or IL-15 Fc fusion proteins, poor accumulation in the target tumor due to short PK, lack of or ineffective tumor targeting, and undesirable accumulation and immune activation activities in normal tissues. Nonetheless, cytokine therapies can be effective, and there is an unmet need in the art to overcome these and other drawbacks.

Accordingly, there remains a need to optimize the pharmacokinetics and/or biological activities of these and other agents. Embodiments of the present disclosure address these problems and more by providing activatable proproteins comprising cytokines that can be activated within a target tissue, for example, a cancer tissue or tumor.

### BRIEF SUMMARY

Embodiments of the present disclosure include an activatable proprotein homodimer, comprising a first polypeptide and a second polypeptide, wherein the first polypeptide and the second polypeptide comprise, in a C- to N-terminal orientation, a binding moiety, a first linker, a cytokine, a second linker, and a cytokine receptor,

wherein the binding moiety of the first polypeptide binds to the binding moiety of the second polypeptide, wherein the cytokine of the first polypeptide binds to the cytokine receptor of the second polypeptide, and wherein the cytokine receptor of the first polypeptide binds to the cytokine of the second polypeptide, wherein said binding masks a binding site of the cytokine that otherwise binds to its wild-type cognate receptor on the surface of an immune cell in vitro or in vivo, and wherein at least one of the first or the second linker is a cleavable linker.

In some embodiments, the cytokine receptor is a variant that comprises one or more amino acid alterations relative to the corresponding wild-type cytokine receptor, and which has reduced binding affinity to the cytokine relative to that of the wild-type cytokine receptor. In some embodiments, the cytokine receptor variant has reduced binding affinity to the cytokine of about or at least about 2-fold, 5-fold, 10-fold, 50-fold, 100-fold, 1000-fold or more, relative to the binding affinity of the wild-type cytokine receptor to the cytokine.

In some embodiments, the cytokine is a variant that comprises one or more amino acid alterations relative to the corresponding wild-type cytokine, and has altered (increased, decreased) binding affinity to its wild-type cognate receptor on the surface of the immune cell in vitro or in vivo. In some embodiments, the cytokine variant has altered (increased, decreased) binding affinity to its wild-type cognate receptor of about or at least about 2-fold, 5-fold, 10-fold, 50-fold, 100-fold, 1000-fold or more, relative to the binding affinity of the wild-type cytokine to the wild-type cognate receptor. In some embodiments, the cytokine receptor comprises, consists, or consists essentially of the extracellular domain (ECD) of the cytokine receptor.

In some embodiments:

the cytokine comprises an interleukin-2 (IL-2) protein, the cytokine receptor comprises an IL-2Rβ protein, and the wild-type cognate receptor comprises IL-2Rβ/λc on the surface of the immune cell;

the cytokine comprises an interleukin-7 (IL-7) protein, the cytokine receptor comprises an IL-7Rα protein, and the wild-type cognate receptor comprises IL-7Rα/λc on the surface of the immune cell;

the cytokine comprises an interleukin-15 (IL-15) protein, the cytokine receptor comprises an IL-15Rβ protein, and the wild-type cognate receptor comprises IL-15Rβ/λc on the surface of the immune cell;

the cytokine comprises an interleukin-21 (IL-21) protein, the cytokine receptor comprises an IL-21R protein, and the wild-type cognate receptor comprises IL-21R/λc on the surface of the immune cell;

the cytokine comprises a type I interferon (IFN) protein, the cytokine receptor comprises a IFNAR2 protein, and the wild-type cognate receptor comprises IFNAR1/IFNAR2 on the surface of the immune cell;

the cytokine comprises a type II IFN protein, the cytokine receptor comprises a IFNGR1 protein, and the wild-type cognate receptor comprises IFNGR1/IFNGR2 on the surface of the immune cell; or

the cytokine comprises a type III IFN protein, the cytokine receptor comprises a IL10Rβ or IFN-λR1 protein, and the wild-type cognate receptor comprises IL10Rβ/IFN-λR1 on the surface of the immune cell.

In some embodiments,

the IL-2 protein or variant thereof comprises, consists, or consists essentially of an amino acid sequence that is at least 80, 85, 90, 95, 98, or 100% identical to a sequence selected from Table S1; and

the IL-2Rβ protein or variant thereof comprises, consists, or consists essentially of an amino acid sequence that is at least 80, 85, 90, 95, 98, or 100% identical to a sequence selected from Table S8.

In some embodiments,

the IL-7 protein or variant thereof comprises, consists, or consists essentially of an amino acid sequence that is at least 80, 85, 90, 95, 98, or 100% identical to a sequence selected from Table S2, and optionally comprises or retains a substitution at E106, as defined by the mature IL-7 sequence, including an E106A substitution; and

the IL-7Rα protein or variant thereof comprises, consists, or consists essentially of an amino acid sequence that is at least 80, 85, 90, 95, 98, or 100% identical to a sequence selected from Table S9, which optionally comprises or retains a substitution at any one or more of I46, K77, and/or L80, as defined by the mature IL-7Rα sequence, optionally an I46T substitution, a K77A substitution, and/or an L80A substitution.

In some embodiments,

the IL-15 protein or variant thereof comprises, consists, or consists essentially of an amino acid sequence that is at least 80, 85, 90, 95, 98, or 100% identical to a sequence selected from Table S3; and

the IL-15Rβ protein or variant thereof comprises, consists, or consists essentially of an amino acid sequence that is at least 80, 85, 90, 95, 98, or 100% identical to a sequence selected from Table S10.

In some embodiments,

the IL-21 protein or variant thereof comprises, consists, or consists essentially of an amino acid sequence that is at least 80, 85, 90, 95, 98, or 100% identical to a sequence selected from Table S4; and

the IL-21R protein or variant thereof comprises, consists, or consists essentially of an amino acid sequence that is at least 80, 85, 90, 95, 98, or 100% identical to a sequence selected from Table S11, which optionally comprises or retains a substitution at any one or more of Y36, M70, D72, D73, and/or Y129, as defined by the mature IL-21R sequence, optionally any one or more of Y36A, M70A, D72A, D73A, and/or Y129A.

In some embodiments, the type I IFN protein is selected from IFNA1, IFNA2, IFNA4, IFNA5, IFNA6, IFNA7, IFNA8, IFNA10, IFNA13, IFNA14, IFNA16, IFNA17, IFNA21, IFNB1, IFNB3, IFNW1, and IFNK, including cytokine variants thereof, optionally wherein the IFNA1, IFNA2, IFNA4, IFNA5, IFNA6, IFNA7, IFNA8, IFNA10, IFNA13, IFNA14, IFNA16, IFNA17, IFNA21, IFNB1, IFNB3, IFNW1, or IFNK protein or variant thereof comprises, consists, or consists essentially of an amino acid sequence that is at least 80, 85, 90, 95, 98, or 100% identical to a sequence selected from Table S5, which is optionally a human IFNA2 variant that comprises or retains a K23 substitution, as defined by the mature IFNA2 sequence, optionally a K23R substitution; and

the IFNAR2 protein or variant thereof comprises, consists, or consists essentially of an amino acid sequence that is at least 80, 85, 90, 95, 98, or 100% identical to a sequence selected from Table S12, which optionally comprises or retains a substitution at M47 and/or E78, as defined by the mature IFNAR sequence, optionally an M47A, M47V, and/or E78A substitution.

In some embodiments, the type II IFN protein is IFNγ, optionally the IFNG1 subunit and/or the IFNG2 subunit, including cytokine variants thereof, optionally wherein the IFNγ protein or variant thereof comprises, consists, or consists essentially of an amino acid sequence that is at least 80, 85, 90, 95, 98, or 100% identical to a sequence selected from Table S6; and

the IFNGR1 protein or variant thereof comprises, consists, or consists essentially of an amino acid sequence that is at least 80, 85, 90, 95, 98, or 100% identical to a sequence selected from Table S13. In some embodiments, the type III IFN protein is IFN-λ, optionally selected from one or more of IFN-λ1, IFN-λ2, IFN-λ3, and IFN-λ4 protein, including cytokine variants thereof.

In some embodiments,

the IFN-λ protein or variant thereof comprises, consists, or consists essentially of an amino acid sequence that is at least 80, 85, 90, 95, 98, or 100% identical to a sequence selected from Table S7; and

the IL10Rβ or IFN-λR1 protein or variant thereof comprises, consists, or consists essentially of an amino acid sequence that is at least 80, 85, 90, 95, 98, or 100% identical to a sequence selected from Table S14.

In some embodiments, the binding moieties do not bind to the cytokine or the cytokine receptor. In some embodiments, the binding moieties of the first polypeptide and the second polypeptide bind together, optionally homodimerize, via at least one non-covalent interaction. In some embodiments, the binding moieties of the first polypeptide and the second polypeptide bind together, optionally homodimerize, via at least one covalent bond. In some embodiments, the at least one covalent bond comprises at least one disulfide bond. In some embodiments, the binding moieties of the first polypeptide and the second polypeptide are selected from Table M1.

In some embodiments, the binding moieties of the first polypeptide and the second polypeptide comprise an antigen binding domain of an immunoglobulin, including antigen binding fragments and variants thereof. In some embodiments, the binding moieties of the first polypeptide and the second polypeptide comprise, consist, or consist essentially of a CH1, CH2, CH3, CH1CH3, CH2CH3, CH1CH2CH3, and/or CL domain of an immunoglobulin, including fragments and variants thereof. In some embodiments, the binding moieties of the first polypeptide and the second polypeptide comprise, in an N- to C-terminal orientation: (1) an antigen binding domain of an immunoglobulin, including antigen binding fragments and variants thereof; and (2) a CH1, CH2, CH3, CH1CH3, CH2CH3, CH1CH2CH3, and/or CL domain of an immunoglobulin, including fragments and variants thereof. In some embodiments, the antigen binding domain comprises a VH or VL domain of an immunoglobulin, including antigen binding fragments and variants thereof. In some embodiments, the binding moieties of the first polypeptide and the second polypeptide do not bind to an antigen. In some embodiments, the binding moieties of the first polypeptide and the second polypeptide comprise a CH2CH3 domain of an immunoglobulin. In some embodiments, the immunoglobulin is from an immunoglobulin class selected from IgG1, IgG2, IgG3, IgG4, IgA, IgD, IgE, and IgM. In some embodiments, the binding moieties of the first polypeptide and the second polypeptide comprise a leucine zipper peptide.

In some embodiments, the cleavable linker comprises a protease cleavage site, optionally wherein the cleavable linker is selected from Table L2. In some embodiments, the protease cleavage site is cleavable by a protease selected from one or more of a metalloprotease, a serine protease, a cysteine protease, and an aspartic acid protease. In some embodiments, the protease cleavage site is cleavable by a protease selected from one or more of MMP1, MMP2, MMP3, MMP4, MMP5, MMP6, MMP7, MMP8, MMP9, MMP10, MMP11, MMP12, MMP13, MMP14, TEV protease, matriptase, uPA, FAP, Legumain, PSA, Kallikrein, Cathepsin A, and Cathepsin B.

In some embodiments, the first linker and/or the second linker are about 1-50 1-40, 1-30, 1-20, 1-10, 1-5, 1-4, 1-3 amino acids in length, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acids in length.

In some embodiments, the first linker is a cleavable linker, and the second linker is a non-cleavable linker. In some embodiments, cleavage, optionally protease cleavage, of the first linker exposes the binding site(s) of the cytokine that binds to its wild-type cognate receptor on the surface of the immune cell in vitro or in vivo. In some embodiments, the first linker is non-cleavable linker, and the second linker is a cleavable linker. In some embodiments, cleavage, optionally protease cleavage, of the second linker exposes the binding site(s) of the cytokine that binds to its wild-type cognate receptor on the surface of the immune cell in vitro or in vivo. In some embodiments, the immune cell is selected from one or more of a T cell, a B cell, a natural killer cell, a monocyte, and a macrophage.

In some embodiments, the first polypeptide and the second polypeptide comprise, consist, or consist essentially of an amino acid sequence that is at least 80, 85, 90, 95, 98, or 100% identical to a sequence selected from Table S15. In some embodiments, the activatable proprotein homodimer is substantially in homodimeric form in a physiological solution, or under physiological conditions, optionally in vivo conditions.

Also included is a recombinant nucleic acid molecule encoding an activatable proprotein homodimer described herein, a vector comprising the recombinant nucleic acid molecule of, and a host cell comprising the recombinant nucleic acid molecule or the vector.

Certain embodiments include methods of producing an activatable proprotein, comprising culturing the host cell described herein under culture conditions suitable for the expression of the activatable proprotein homodimer, and isolating the activatable proprotein from the culture.

Some embodiments include a pharmaceutical composition, comprising an activatable proprotein homodimer described herein, and a pharmaceutically acceptable carrier.

Also included are methods of treating disease in a subject, comprising administering to the subject a therapeutically effective amount of a pharmaceutical composition described herein. In some embodiments, the disease is selected from one or more of a cancer, a viral infection, and an immune disorder. In some embodiments, the cancer is a primary cancer or a metastatic cancer, and is selected from one or more of melanoma (optionally metastatic melanoma), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer.

In some embodiments, following administration, the activatable proprotein homodimer is activated through protease cleavage in a cell or tissue, optionally a cancer cell or cancer tissue, which exposes the binding site(s) of the cytokine that binds its wild-type cognate receptor on the surface of the immune cell in vitro or in vivo, and thereby generates an activated protein.

In some embodiments:

the activated protein binds via the IL-7 protein to IL-7Rα/λc on the surface of the immune cell;

the activated protein binds via the IL-21 protein to IL-21R/λc on the surface of the immune cell;

the activated protein binds via the type I IFN protein to IFNAR1/IFNAR2 on the surface of the immune cell;

the activated protein binds via the type II IFN protein to IFNGR1/IFNGR2 on the surface of the immune cell; or

the activated protein binds via the type III IFN protein to IL10Rβ/IFN-λR1 on the surface of the immune cell.

In some embodiments, the immune cell is selected from one or more of a T cell, a B cell, a natural killer cell, a monocyte, and a macrophage. In some embodiments, administration and activation of the activatable proprotein increases an immune response in the subject by about or at least about 5, 10, 15, 20, 25, 30, 35, 40, 45, 50, 60, 70, 80, 90, 100, 200, 300, 400, 500, 600, 700, 800, 900, 1000, 2000/o or more, relative to a control, optionally wherein the immune response is an anti-cancer or anti-viral immune response. In some embodiments, administration and activation of the activatable proprotein increases cell-killing in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control, optionally wherein the cell-killing is cancer cell-killing or virally-infected cell-killing.

In some embodiments, the viral infection is selected from one or more of human immunodeficiency virus (HIV), Hepatitis A, Hepatitis B, Hepatitis C, Hepatitis E, Caliciviruses associated diarrhoea, Rotavirus diarrhoea, Haemophilus influenzae B pneumonia and invasive disease, influenza, measles, mumps, rubella, Parainfluenza associated pneumonia, Respiratory syncytial virus (RSV) pneumonia, Severe Acute Respiratory Syndrome (SARS), Human papillomavirus, Herpes simplex type 2 genital ulcers, Dengue Fever, Japanese encephalitis, Tick-borne encephalitis, West-Nile virus associated disease, Yellow Fever, Epstein-Barr virus, Lassa fever, Crimean-Congo haemorrhagic fever, Ebola haemorrhagic fever, Marburg haemorrhagic fever, Rabies, Rift Valley fever, Smallpox, upper and lower respiratory infections, and poliomyelitis, optionally wherein the subject is HIV-positive.

In some embodiments, the immune disorder is selected from one or more of type 1 diabetes, vasculitis, and an immunodeficiency.

In some embodiments, the pharmaceutical composition is administered to the subject by parenteral administration. In some embodiments, the parenteral administration is intravenous administration.

Some embodiments include the use of a pharmaceutical composition described herein in the preparation of a medicament for treating a disease in a subject.

### BRIEF DESCRIPTION OF THE FIGURES

FIG. 1 provides a key to the structural features of exemplary activatable procytokine homodimers.

FIG. 2 provides the structural features of an exemplary procytokine homodimer, which is composed of an Fc domain with hinge, a flexible/cleavable linker, a cytokine, a flexible/non-cleavable linker, and a wild-type cytokine receptor (or ECD of the receptor). The procytokine has very low activity before protease cleavage, and somewhat low but significantly increased activity after protease cleavage.

FIG. 3 provides the structural features of an exemplary procytokine homodimer, which is composed of an Fc domain with hinge, a flexible/cleavable linker, a cytokine, a flexible/non-cleavable linker, and a cytokine receptor variant (or ECD of the variant) that binds to the cytokine with lower affinity than the wild-type cytokine receptor. The procytokine has low activity before protease cleavage, and medium to high activity after protease cleavage.

FIG. 4 illustrates the interactions between the activated procytokines from FIGS. 2-3 (center and right) and their wild-type cognate receptors on the surface of a cell, relative to that of a standalone cytokine (left).

FIG. 5A illustrates the IL-7/IL-7Rα and IL-21/IL-221R interactions on the surface of a cell, and FIG. 5B illustrates IL-2/IL-2R and IL-15 and IL-15R interactions on the surface of a cell.

FIG. 6 illustrates the various IFN/IFNR interactions on the surface of a cell.

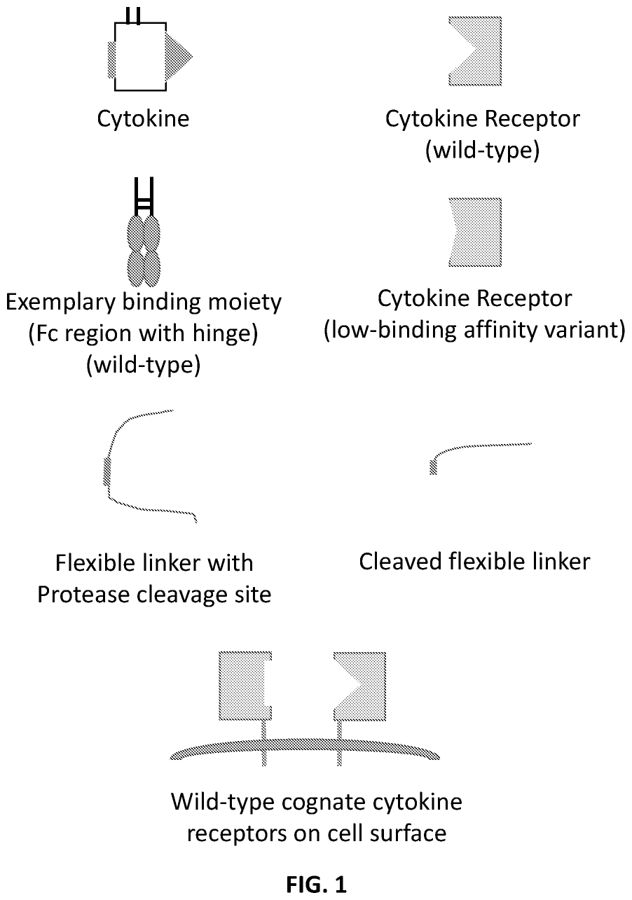

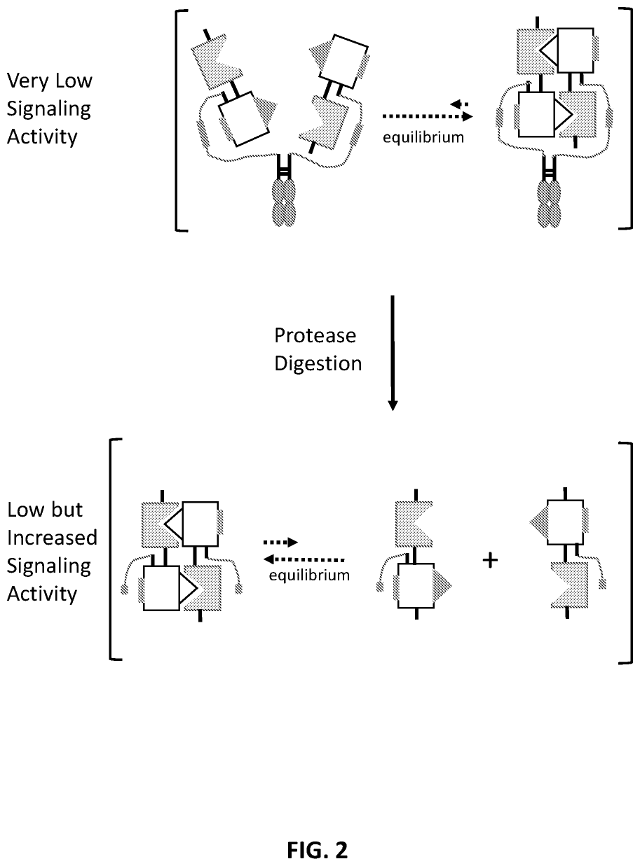

### DETAILED DESCRIPTION

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

For the purposes of the present disclosure, the following terms are defined below.

The articles “a” and “an” are used herein to refer to one or to more than one (i.e., to at least one) of the grammatical object of the article. By way of example, “an element” includes “one element”, “one or more elements” and/or “at least one element”.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The terms “activatable proprotein,” “activatable prodrug”, “prodrug” or “proprotein” are used interchangeably herein and refer to an activatable proprotein comprising at least a masking moiety and an active domain, or derivatives/variants therefrom, as described herein. In one embodiment, the proprotein may also comprise one or more protein domains.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes. As used herein, the term “antigen” includes substances that are capable, under appropriate conditions, of inducing an immune response to the substance and of reacting with the products of the immune response. More broadly, the term “antigen” includes any substance to which an antibody binds, or for which antibodies are desired, regardless of whether the substance is immunogenic. For such antigens, antibodies can be identified by recombinant methods, independently of any immune response.

An “antagonist” refers to biological structure or chemical agent that interferes with or otherwise reduces the physiological action of another agent or molecule. In some instances, the antagonist specifically binds to the other agent or molecule. Included are full and partial antagonists.

An “agonist” refers to biological structure or chemical agent that increases or enhances the physiological action of another agent or molecule. In some instances, the agonist specifically binds to the other agent or molecule. Included are full and partial agonists.

As used herein, the term “amino acid” is intended to mean both naturally occurring and non-naturally occurring amino acids as well as amino acid analogs and mimetics. Naturally-occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

As used herein, a subject “at risk” of developing a disease, or adverse reaction may or may not have detectable disease, or symptoms of disease, and may or may not have displayed detectable disease or symptoms of disease prior to the treatment methods described herein. “At risk” denotes that a subject has one or more risk factors, which are measurable parameters that correlate with development of a disease, as described herein and known in the art. A subject having one or more of these risk factors has a higher probability of developing disease, or an adverse reaction than a subject without one or more of these risk factor(s).

“Biocompatible” refers to materials or compounds which are generally not injurious to biological functions of a cell or subject and which will not result in any degree of unacceptable toxicity, including allergenic and disease states.

The term “binding” refers to a direct association between two molecules, due to, for example, covalent, electrostatic, hydrophobic, and ionic and/or hydrogen-bond interactions, including interactions such as salt bridges and water bridges.

By “coding sequence” is meant any nucleic acid sequence that contributes to the code for the polypeptide product of a gene. By contrast, the term “non-coding sequence” refers to any nucleic acid sequence that does not directly contribute to the code for the polypeptide product of a gene.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “endotoxin free” or “substantially endotoxin free” relates generally to compositions, solvents, and/or vessels that contain at most trace amounts (e.g., amounts having no clinically adverse physiological effects to a subject) of endotoxin, and preferably undetectable amounts of endotoxin. Endotoxins are toxins associated with certain micro-organisms, such as bacteria, typically gram-negative bacteria, although endotoxins may be found in gram-positive bacteria, such as Listeria monocytogenes. The most prevalent endotoxins are lipopolysaccharides (LPS) or lipo-oligo-saccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C. and a holding time of 30 minutes is often sufficient to achieve a 3 log reduction in endotoxin levels. Other methods of removing endotoxins are contemplated, including, for example, chromatography and filtration methods, as described herein and known in the art.

Endotoxins can be detected using routine techniques known in the art. For example, the Limulus Amoebocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/mg of active compound. Typically, 1 ng lipopolysaccharide (LPS) corresponds to about 1-10 EU.

The term “half maximal effective concentration” or “EC₅₀” refers to the concentration of an agent (e.g., activatable proprotein) as described herein at which it induces a response halfway between the baseline and maximum after some specified exposure time; the EC₅₀ of a graded dose response curve therefore represents the concentration of a compound at which 50% of its maximal effect is observed. EC50 also represents the plasma concentration required for obtaining 50% of a maximum effect in vivo. Similarly, the “EC₉₀” refers to the concentration of an agent or composition at which 90% of its maximal effect is observed. The “EC₉₀” can be calculated from the “EC50” and the Hill slope, or it can be determined from the data directly, using routine knowledge in the art. In some embodiments, the EC₀ of an agent (e.g., activatable proprotein) is less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, an agent will have an EC₅₀ value of about 1 nM or less.

“Immune response” means any immunological response originating from immune system, including responses from the cellular and humeral, innate and adaptive immune systems. Exemplary cellular immune cells include for example, lymphocytes, macrophages, T cells, B cells, NK cells, neutrophils, eosinophils, dendritic cells, mast cells, monocytes, and all subsets thereof. Cellular responses include for example, effector function, cytokine release, phagocytosis, efferocytosis, translocation, trafficking, proliferation, differentiation, activation, repression, cell-cell interactions, apoptosis, etc. Humeral responses include for example IgG, IgM, IgA, IgE, responses and their corresponding effector functions.

The “half-life” of an agent such as an activatable proprotein can refer to the time it takes for the agent to lose half of its pharmacologic, physiologic, or other activity, relative to such activity at the time of administration into the serum or tissue of an organism, or relative to any other defined time-point. “Half-life” can also refer to the time it takes for the amount or concentration of an agent to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 1.5, 2, 3, 4, 5, 6, 7, 8, 9, 10, 15, 20, 30, 40, 50, 60, 70, 80, 90, 100 or more times (e.g., 500, 1000 times) (including all integers and ranges in between e.g., 1.5, 1.6, 1.7. 1.8, etc.) the amount produced by no composition (e.g., the absence of agent) or a control composition. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease (including all integers and ranges in between) in the amount produced by no composition (e.g., the absence of an agent) or a control composition. Examples of comparisons and “statistically significant” amounts are described herein.

The terms “polypeptide,” “protein” and “peptide” are used interchangeably and mean a polymer of amino acids not limited to any particular length. The term “enzyme” includes polypeptide or protein catalysts. The terms include modifications such as myristoylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. In certain embodiments, the polypeptide is a “recombinant” polypeptide, produced by recombinant cell that comprises one or more recombinant DNA molecules, which are typically made of heterologous polynucleotide sequences or combinations of polynucleotide sequences that would not otherwise be found in the cell.

The term “polynucleotide” and “nucleic acid” includes mRNA, RNA, cRNA, cDNA, and DNA. The term typically refers to polymeric form of nucleotides of at least 10 bases in length, either ribonucleotides or deoxynucleotides or a modified form of either type of nucleotide. The term includes single and double stranded forms of DNA. The terms “isolated DNA” and “isolated polynucleotide” and “isolated nucleic acid” refer to a molecule that has been isolated free of total genomic DNA of a particular species. Therefore, an isolated DNA segment encoding a polypeptide refers to a DNA segment that contains one or more coding sequences yet is substantially isolated away from, or purified free from, total genomic DNA of the species from which the DNA segment is obtained. Also included are non-coding polynucleotides (e.g., primers, probes, oligonucleotides), which do not encode a polypeptide. Also included are recombinant vectors, including, for example, expression vectors, viral vectors, plasmids, cosmids, phagemids, phage, viruses, and the like.

Additional coding or non-coding sequences may, but need not, be present within a polynucleotide described herein, and a polynucleotide may, but need not, be linked to other molecules and/or support materials. Hence, a polynucleotide or expressible polynucleotides, regardless of the length of the coding sequence itself, may be combined with other sequences, for example, expression control sequences.

The term “isolated” polypeptide or protein referred to herein means that a subject protein (1) is free of at least some other proteins with which it would typically be found in nature, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or non-covalent interaction) with portions of a protein with which the “isolated protein” is associated in nature, (6) is operably associated (by covalent or non-covalent interaction) with a polypeptide with which it is not associated in nature, or (7) does not occur in nature. Such an isolated protein can be encoded by genomic DNA, cDNA, mRNA or other RNA, of may be of synthetic origin, or any combination thereof. In certain embodiments, the isolated protein is substantially free from proteins or polypeptides or other contaminants that are found in its natural environment that would interfere with its use (therapeutic, diagnostic, prophylactic, research or otherwise).

In certain embodiments, the “purity” of any given agent (e.g., activatable proprotein) in a composition may be defined. For instance, certain compositions may comprise an agent such as a polypeptide agent that is at least 80%, 85%, 90%, 91%, 92%, 93%, 94%, 95%, 96%, 97%, 98%, 99%, or 100% pure on a protein basis or a weight-weight basis, including all decimals and ranges in between, as measured, for example and by no means limiting, by high performance liquid chromatography (HPLC), a well-known form of column chromatography used frequently in biochemistry and analytical chemistry to separate, identify, and quantify compounds.

The term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

Certain embodiments include biologically active “variants” and “fragments” of the proteins/polypeptides described herein, and the polynucleotides that encode the same. “Variants” contain one or more substitutions, additions, deletions, and/or insertions relative to a reference polypeptide or polynucleotide (see, e.g., the Tables and the Sequence Listing). A variant polypeptide or polynucleotide comprises an amino acid or nucleotide sequence with at least about 50%, 55%, 60%, 65%, 70%, 75%, 80%, 85%, 90%, 91%, 92%, 93%, 94%, 95%, 96%, 97%, 98%, 99% or more sequence identity or similarity or homology to a reference sequence, as described herein, and substantially retains the activity of that reference sequence. Also included are sequences that consist of or differ from a reference sequences by the addition, deletion, insertion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150 or more amino acids or nucleotides and which substantially retain at least one activity of that reference sequence. In certain embodiments, the additions or deletions include C-terminal and/or N-terminal additions and/or deletions.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997.

The term “solubility” refers to the property of an agent (e.g., activatable proprotein) provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, pH 7.4, pH 7.6, pH 7.8, or pH 8.0 (e.g., about pH 5-8). In certain embodiments, solubility is measured in water or a physiological buffer such as PBS or NaCl (with or without NaPO₄). In specific embodiments, solubility is measured at relatively lower pH (e.g., pH 6.0) and relatively higher salt (e.g., 500 mM NaCl and 10 mM NaPO₄). In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90 or 100 mg/ml at room temperature or at 37° C.

A “subject” or a “subject in need thereof” or a “patient” or a “patient in need thereof” includes a mammalian subject such as a human subject.

“Substantially” or “essentially” means nearly totally or completely, for instance, 95%, 96%, 97%, 98%, 99% or greater of some given quantity.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Therapeutic response” refers to improvement of symptoms (whether or not sustained) based on administration of one or more therapeutic agents.

As used herein, the terms “therapeutically effective amount”, “therapeutic dose,” “prophylactically effective amount,” or “diagnostically effective amount” is the amount of an agent (e.g., activatable proprotein, activated protein) needed to elicit the desired biological response following administration.

As used herein, “treatment” of a subject (e.g., a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

Each embodiment in this specification is to be applied to every other embodiment unless expressly stated otherwise.

Activatable Proprotein Homodimers

Embodiments of the present disclosure relate to activatable proprotein homodimers, or procytokines, comprising cytokine proteins that remain relatively inactive in the proprotein form (or prodrug), and which can be “activated” upon contact with the appropriate environment. The activatable proproteins described herein comprise at least two identical (or nearly identical) but separate polypeptide chains, which bind together via non-covalent and/or certain covalent bonds, for example, disulfide bonds, but not via peptide or amide bonds. Generally, each polypeptide chain comprises, in a C- to N-terminal orientation, a binding moiety, a first linker, a cytokine, a second linker, and a corresponding cytokine receptor. The combined binding between the cytokine proteins and their corresponding cytokine receptors sterically hinders the cytokine proteins from interacting with certain of their cognate receptor(s) on a cell. Each polypeptide chain also comprises a binding moiety, which binds to the binding moiety in the other chain and further stabilizes the interaction that sterically hinders the cytokine proteins from interacting with or binding to their cognate receptor(s) on a cell. Typically, at least one of linkers comprises a cleavable linker, which upon cleavage in a target tissue releases at least part of the steric hindrance and restores or increases cytokine protein activity by exposing at least one active or binding site of the cytokine proteins. Such allows the cytokine portion(s) of the now activated protein to interact with or bind to certain of their cognate receptor(s) on an immune cell, and thereby effect downstream immune cell-signaling pathways.

Embodiments of the present disclosure thus include an activatable proprotein homodimer (complex), comprising a first polypeptide (chain) and a second polypeptide (chain), wherein:

the first polypeptide and the second polypeptide comprise, in a C- to N-terminal orientation, a binding moiety, a first linker, a cytokine, a second linker, and a cytokine receptor,

wherein the binding moiety of the first polypeptide binds to the binding moiety of the second polypeptide, wherein the cytokine of the first polypeptide binds to the cytokine receptor of the second polypeptide, and wherein the cytokine receptor of the first polypeptide binds to the cytokine of the second polypeptide, wherein said binding masks a binding site of the cytokine that otherwise binds to its wild-type cognate receptor on the surface of an immune cell in vitro or in vivo, and wherein at least one of the first or the second linker is a cleavable linker.

In some embodiments, the cytokine proteins and the cytokine receptor proteins interact or bind together, for example, via non-covalent or certain covalent bonds (e.g., disulfide bonds). In some instances, the binding of the cytokine proteins to the cytokine receptor proteins sterically blocks or hinders binding of the cytokine proteins to their cognate wild-type receptors expressed on an immune cell.

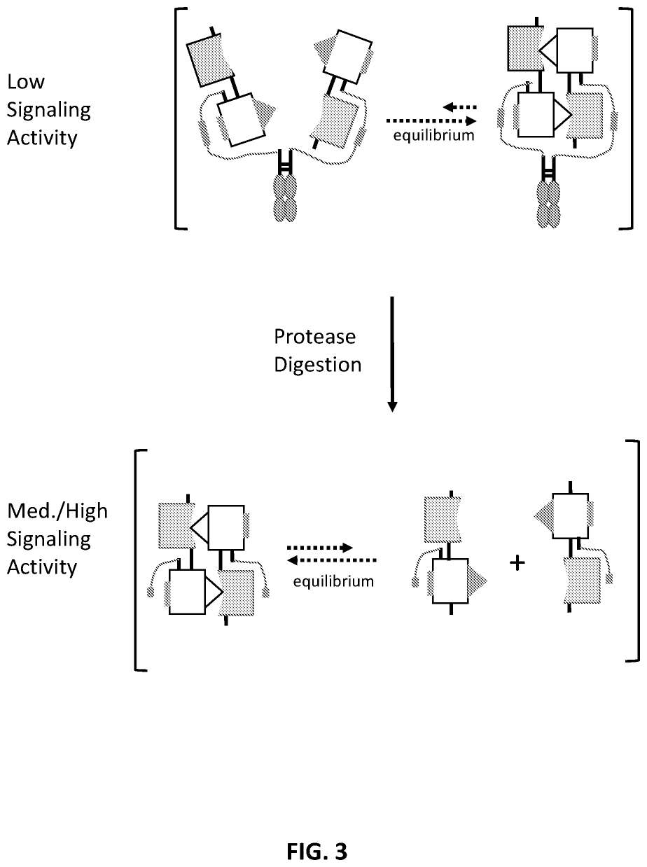

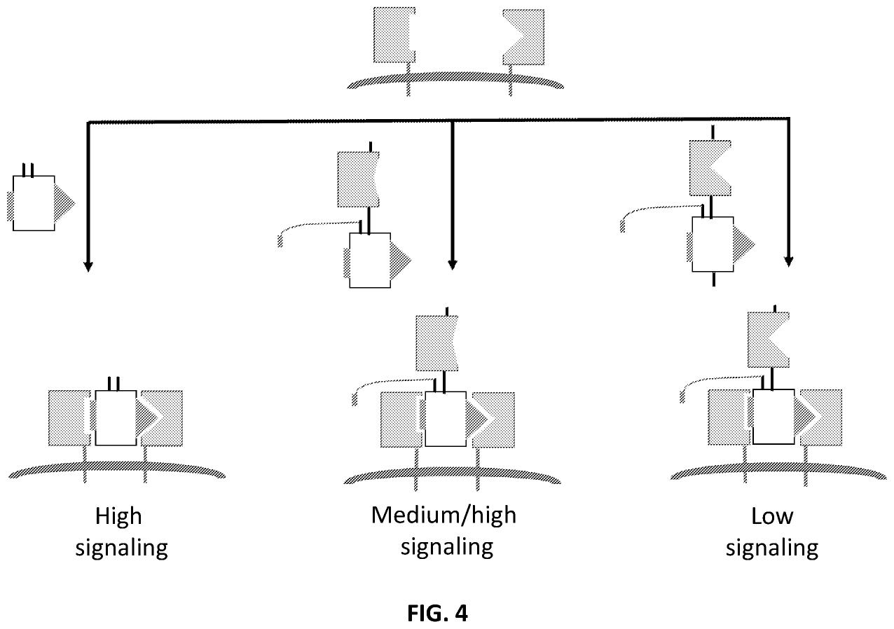

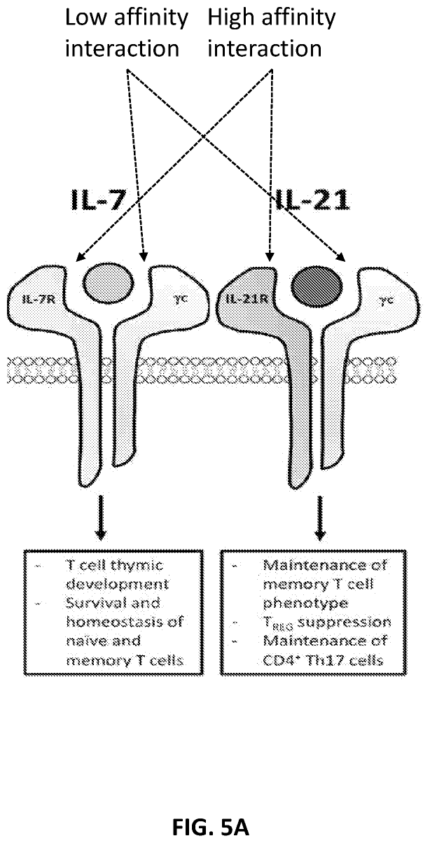

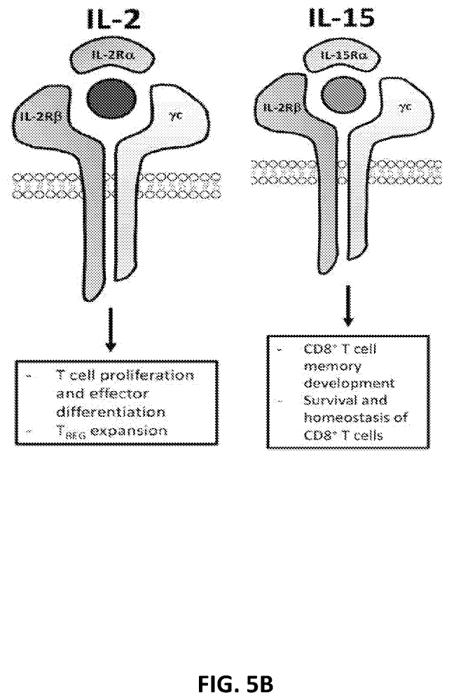

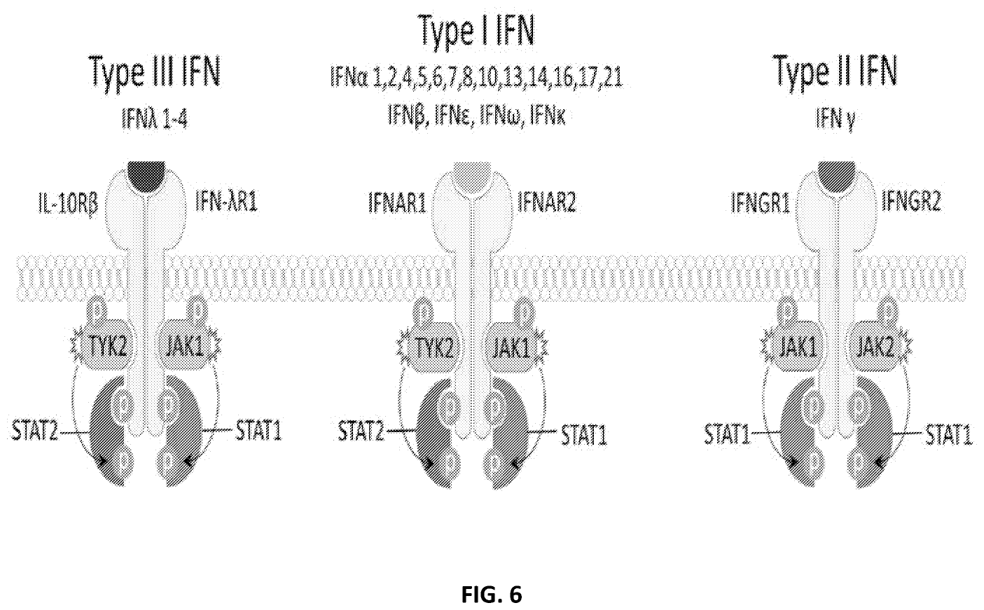

In some embodiments, the cytokine receptor is a variant that comprises one or more amino acid alterations relative to the corresponding wild-type cytokine receptor, and has reduced binding affinity to the cytokine relative to that of the wild-type cytokine receptor. In these and related embodiments, upon cleavage of a linker and release of the proprotein homodimer structure (see FIGS. 3-4), the cytokine will be less likely to continue interacting with the cytokine receptor variant and more likely to bind to its wild-type cognate receptor on the surface of an immune cell. This strategy increases the relative activity of the “activated” cytokine portion of the proprotein.

In some instances, the binding moieties of the first and second polypeptides dimerize together via at least one non-covalent interaction, at least one covalent bond (for example, at least one disulfide bond), or any combination of non-covalent interactions and covalent bonds, to further stabilize the activatable proprotein and/or to further mask the binding of the cytokine proteins to their cognate wild-type receptors on a cell. Typically, however, binding moieties of the first and second polypeptide do not bind together or dimerize via a peptide or amide bond. In some embodiments, the binding moieties bind together as a heterodimer, that is, a heterodimer composed of two different binding moieties. In some embodiments, the binding moieties bind together as a homodimer, that is, a homodimer composed of two identical or nearly identical binding moieties. Thus, the binding moieties of the first and second polypeptides can be the same (or substantially the same) or different. In most instances, the binding moieties of the first and second polypeptides are the same, and do not bind to the cytokine proteins or the cytokine receptor proteins. However, in some instances, one or both of the binding moieties can bind to any one or more of the cytokine proteins or cytokine receptor proteins. Exemplary binding moieties are described herein.

As noted above, at least one of the linkers comprises a cleavable linker, for example, a linker cleavable by a protease. In some instances, one linker comprises a cleavable linker and the other linker is a stable (e.g., physiologically stable) linker, for example, a flexible linker. In some instances, both linkers comprise cleavable linkers. In some instances, the protease is expressed in target tissues or cells, for example, cancer tissues or cancer cells. Cleavage of the linker in that context releases removes the steric hindrance of the cytokine proteins, and allows selective activation of the cytokine proteins in diseased tissues or cells, relative to normal or healthy tissues or cells. Such selective and localized activation not only reduces needless consumption of administered cytokine proteins, thereby increasing their half-life, but also enhances tissue penetration and reduces undesirable systemic effects of cytokines, among other advantages. Exemplary linkers are described herein.

In some embodiments, the homodimeric binding between the first and second polypeptides allosterically inhibits the binding of the cytokine proteins to their cognate wild-type receptors on the surface of an immune cell. In these and related embodiments, the cytokine protein portion of the activatable proprotein homodimer shows no binding or substantially no binding to its wild-type cognate receptor, for example, in vitro or on the surface of an immune cell, or no more than 0.001%, 0.01%, 0.1%, 1%, 2%, 3%, 4%, 5%, 6%, 7%, 8%, 9%, 10%1, 15%, 20%, 25%, 30%, 35%, 40%, or 50% binding to its target, as compared to the binding of the corresponding cytokine protein alone, optionally for at least 2, 4, 6, 8, 12, 28, 24, 30, 36, 48, 60, 72, 84, 96 hours, or 5, 10, 15, 30, 45, 60, 90, 120, 150, 180 days, or 1, 2, 3, 4, 5, 6, 7, 8, 9, 10, 11, 12 months or greater, optionally as measured in vivo or in a Target Displacement in vitro assay available in the art.

Certain activatable proproteins are composed only of two of the foregoing protein chains, that is, they are composed only of a first polypeptide and a second polypeptide, as described herein. In some instances, however, certain activatable proproteins comprise multiple chains, for example, where the first and second polypeptide chains form a “core structure” upon which additional or higher-order structures can be built, the various core structures being optionally bound together via additional protein binding domains.

The individual components of the activatable proprotein homodimers are described in greater detail herein.

Cytokines. The activatable proprotein homodimers described herein comprise at least one cytokine or cytokine protein, including wild-type cytokines and active variants and fragments thereof. In certain embodiments, the cytokine comprises a human cytokine sequence. Exemplary cytokines include interleukin-2 (IL-2), interleukin-7 (IL-7), interleukin-15 (IL-15), interleukin-21 (IL-21), and the interferons (IFNs) such as type I IFNs, type II IFNs, and type III IFNs, as described herein.

In some embodiments, the cytokine is a variant that comprises one or more amino acid alterations relative to the corresponding wild-type cytokine, and has altered (e.g., increased, decreased) binding affinity to its wild-type cognate receptor on the surface of the immune cell in vitro or in vivo. For example, in some embodiments, the cytokine variant has increased binding affinity to its wild-type cognate receptor of about or at least about 2-fold, 5-fold, 10-fold, 50-fold, 100-fold, 1000-fold or more, relative to the binding affinity of the wild-type cytokine to the wild-type cognate receptor. In certain embodiments, the cytokine variant has decreased binding affinity to its wild-type cognate receptor of about or at least about 2-fold, 5-fold, 10-fold, 50-fold, 100-fold, 1000-fold or more, relative to the binding affinity of the wild-type cytokine to the wild-type cognate receptor.

Interleukin-2 (IL-2). In certain embodiments, the cytokine is IL-2, and the wild-type cognate receptor comprises IL-2Rβ/λc on the surface of an immune cell. IL-2 is a cytokine that signals through the IL-2 receptor (IL-2R), a complex composed of up to three chains, termed the α (CD25), β (CD122) and γc (CD132) chains. IL-2 is produced by T-cells in response to antigenic or mitogenic stimulation, and is required for T-cell proliferation and other activities crucial to regulation of the immune response. IL-2 can stimulate B-cells, monocytes, lymphokine-activated killer cells, natural killer cells, and glioma cells, among other immune cells.

IL-2 is a 15-16 kDA protein composed of a signal peptide (residues 1-20) and an active mature protein (residues 21-153). Exemplary human IL-2 protein sequences are provided in Table S1.

TABLE S1
Exemplary IL-2 Protein sequences

| | | SEQ ID |
|---|---|---|
| Name | Sequence | NO: |
| Human IL-2 | MYRMQLLSCIALSLALVTNSAPTSSSTKKTQLQLEHLLLDLQMILNGINN | 1 |
| FL | YKNPKLTRMLTFKFYMPKKATELKHLQCLEEELKPLEEVLNLAQSKNFHL | |
| Precursor | RPRDLISNINVIVLELKGSETTFMCEYADETATIVEFLNRWITFCQSIIS | |
| | TLT | |
| Human IL-2 | APTSSSTKKTQLQLEHLLLDLQMILNGINNYKNPKLTRMLTFKFYMPKKA | 2 |
| mature form | TELKHLQCLEEELKPLEEVLNLAQSKNFHLRPRDLISNINVIVLELKGSE | |
| (C125S) | TTFMCEYADETATIVEFLNRWITFSQSIISTLT | |
| Human IL-2 | APTSSSTKKTQLQLEHLLLDLQMILNGINNYKNPKLTRMLTFKFYMPKKA | 3 |
| mature form | TELKHLQCLEEELKPLEEVLNLAHSKNFHFDPRDWSNINVFVLELKGSE | |
| (D10) | TTFMCEYADETATIVEFLNRWITFCQSIISTLT | |
| Q74H, L80F, | | |
| R81D, L85V, | | |
| I86V, and | | |
| I92F | | |
| Human IL-2 | APASSSTKKTQLQLEHLLLDLQMILNGINNYKNPKLTRMLTFKFYMPKKA | 4 |
| mature form | TELKHLQCLEEELKPLEEVLNLAQSKNFHLRPRDLISNINVIVLELKGSE | |
| with T3A | TTFMCEYADETATIVEFLNRWITFSQSIISTLT | |
| Human IL-2 | APASSSTKKTQLQLEHLLLDLQMILNGINNYKNPKLTRMLTAKFYMPKKA | 5 |
| mature form | TELKHLQCLEEELKPLEEVLNLAQSKNFHLRPRDLISNINVIVLELKGSE | |
| with T3A, | TTFMCEYADETATIVEFLNRWITFSQSIISTLT | |
| F42A | | |
| Human IL-2 | APASSSTKKTQLQLEHLLLDLQMILNGINNYKNPKLTRMLTFKFYMPKKA | 6 |
| mature form | TELKHLQCLEEELKPLEEALNLAPSKNFHLRPRDLISNINVIVLELKGSE | |
| with T3A, | TTFMCEYADETATIVEFLNRWITFSQSTISTLT | |
| V69A, Q74P, | | |
| I128T | | |
| Human IL-2 | APASSSTKKTQLQLEHLLLTLQMILNGINNYKNPKLTRMLTFKFYMPKKA | 7 |
| mature form | TELKHLQCLEEELKPLEEVLNLAQSKNFHLRPRDLISNINVIVLELKGSE | |
| with T3A, | TTFMCEYADETATIVEFLNRWITFSQSIISTLT | |
| D20T | | |
| Human IL-2 | APASSSTKKTQLQLEHLLLTLQMILNGINNYKNPKLTRMLTAKFYMPKKA | 8 |
| mature form | TELKHLQCLEEELKPLEEVLNLAQSKNFHLRPRDLISNINVIVLELKGSE | |
| with T3A, | TTFMCEYADETATIVEFLNRWITFSQSIISTLT | |
| D20T, F42A | | |
| Human IL-2 | APASSSTKKTQLQLEHLLLDLQMILNGINNYKNPKLTEMLTFKFYMPKKA | 9 |
| mature form | TELKHLQCLEEELKPLEEVLNLAQSKNFHLRPRDLISNINVIVLELKGSE | |
| with R38E, | TTFMCEYADETATIVEFLNRWITFSQSIISTLT | |
| T3A | | |
| Human IL-2 | APASSSTKKTQLQLEHLLLDLQMILNGINNYKNPKLTRMLTFKFYMPKKA | 10 |
| mature form | TELKHLQCLEKELKPLEEVLNLAQSKNFHLRPRDLISNINVIVLELKGSE | |
| with E61K, | TTFMCEYADETATIVEFLNRWITFSQSIISTLT | |
| T3A | | |
| Human IL-2 | APASSSTKKTQLQLEHLLLDLQMILNGINNYKNPKLTRMLTFKFYMPKKA | 11 |
| mature form | TELKHLQCLEEKLKPLEEVLNLAQSKNFHLRPRDLISNINVIVLELKGSE | |
| with E62K, | TTFMCEYADETATIVEFLNRWITFSQSIISTLT | |
| T3A | | |
| Human IL-2 | APASSSTKKTQLQLEHLLLDLQMILNGINNYKNPKLTRMLTFKFYMPKKA | 12 |
| mature form | TELKHLQCLEKKLKPLEEVLNLAQSKNFHLRPRDLISNINVIVLELKGSE | |
| with E61K, | TTFMCEYADETATIVEFLNRWITFSQSIISTLT | |
| E62K, T3A | | |
| Human IL-2 | APASSSTKKTQLQLEHLLLDLQMILNGINNYKNPKLTEMLTFKFYMPKKA | 13 |
| mature form | TELKHLQCLEKKLKPLEEVLNLAQSKNFHLRPRDLISNINVIVLELKGSE | |
| with R38E, | TTFMCEYADETATIVEFLNRWITFSQSIISTLT | |
| E61K, E62K, | | |
| T3A | | |
| Human IL-2 | APASSSTKKTQLQLEHLLLDLQMILNGINNYKNPKLTEMLTAKFYMPKKA | 14 |
| mature form | TELKHLQCLEEELKPLEEVLNLAQSKNFHLRPRDLISNINVIVLELKGSE | |
| with F42A, | TTFMCEYADETATIVEFLNRWITFSQSIISTLT | |
| R38E, T3A | | |
| Human IL-2 | APASSSTKKTQLQLEHLLLDLQMILNGINNYKNPKLTRMLTAKFYMPKKA | 15 |
| mature form | TELKHLQCLEKELKPLEEVLNLAQSKNFHLRPRDLISNINVIVLELKGSE | |
| with F42A, | TTFMCEYADETATIVEFLNRWITFSQSIISTLT | |
| E61K, T3A | | |
| Human IL-2 | APASSSTKKTQLQLEHLLLDLQMILNGINNYKNPKLTRMLTAKFYMPKKA | 16 |
| mature form | TELKHLQCLEEKLKPLEEVLNLAQSKNFHLRPRDLISNINVIVLELKGSE | |
| with F42A, | TTFMCEYADETATIVEFLNRWITFSQSIISTLT | |
| E62K, T3A | | |
| Human IL-2 | APASSSTKKTQLQLEHLLLDLQMILNGINNYKNPKLTRMLTAKFYMPKKA | 17 |
| mature form | TELKHLQCLEKKLKPLEEVLNLAQSKNFHLRPRDLISNINVIVLELKGSE | |
| with F42A, | TTFMCEYADETATIVEFLNRWITFSQSIISTLT | |
| E61K, E62K, | | |
| T3A | | |
| Human IL-2 | APASSSTKKTQLQLEHLLLDLQMILNGINNYKNPKLTEMLTAKFYMPKKA | 18 |
| mature form | TELKHLQCLEKKLKPLEEVLNLAQSKNFHLRPRDLISNINVIVLELKGSE | |
| with F42A, | TTFMCEYADETATIVEFLNRWITFSQSIISTLT | |
| R38E, E61K, | | |
| E62K, T3A | | |

Thus, in certain embodiments, an IL-2 protein comprises, consists, or consists essentially of an amino acid sequence selected from Table S1, or an active variant or fragment thereof that is at least 80, 85, 90, 95, 98, or 10000 identical to an amino acid sequence selected from Table S1, and binds to and signals through IL-2Rβ/γc and/or IL-2Rα/β/γc receptors.

In some embodiments, an “active” IL-2 protein or fragment or variant is characterized, for example, by its ability to bind to an IL-2Rβ/γc and/or IL-2Rα/β/γc receptor chain present on the surface of an immune cell in vitro or in vivo, and stimulate downstream signaling activities, absent steric hindrance by the binding moieties described herein. Examples of downstream signaling activities include IL-2 mediated signaling via one or more of the JAK-STAT, PI3K/Akt/mTOR, and MAPK/ERK pathways, including combinations thereof. Altogether, IL-2 signaling stimulates an array of downstream pathways leading to responses that have a significant role in the development, function, and survival of CD4 T cells, CD8 T cells, NK cells, NKT cells, macrophages, and intestinal intraepithelial lymphocytes, among others.

In particular embodiments, the IL-2 protein is a mature form of IL-2, or an active variant or fragment thereof, which comprises, consists, or consists essentially of an amino acid sequence that is at least 80, 85, 90, 95, 98, or 1000% identical to amino acids 21-153 of SEQ ID NO: 1. In some embodiments, the IL-2 protein comprises a C145X substitution, as defined by SEQ ID NO: 1, wherein X is any amino acid. In specific embodiments, the IL-2 protein comprises a C145S substitution as defined by SEQ ID NO: 1.

Certain IL-2 proteins comprise, consist, or consist essentially of an amino acid sequence that is at least 80, 85, 90, 95, 98, or 100% identical to SEQ ID NO: 2 (mature human IL-2 with C125S substitution). In some embodiments, an active variant or fragment of SEQ ID NO: 2 retains the S125 residue as defined therein. In some embodiments, the IL-2 protein comprises, consists, or consists essentially of an amino acid sequence that is at least 80, 85, 90, 95, 98, or 100% identical to SEQ ID NO: 3 (mature human IL-2 “D10” variant), optionally wherein the IL-2 protein retains any one or more of the Q74H, L80F, R81D, L85V, I86V, and/or I92F substitutions as defined by SEQ ID NO: 3.

Certain IL-2 proteins comprise one or more amino acid substitutions relative to the exemplary amino acid sequences in Table S1. In certain embodiments, the IL-2 protein is a variant that has increased binding affinity to the wild-type IL-2Rβ/λc complex (e.g., in an in vitro binding assay, on the surface of an immune cell) of about or at least about 2-fold, 5-fold, 10-fold, 50-fold, 100-fold, 1000-fold or more, relative to the binding affinity of the wild-type IL-2 protein to the wild-type IL-2Rβ/γc complex. In certain embodiments, the IL-2 protein is a variant that has reduced binding affinity to the wild-type IL-2Rβ/γc complex (e.g., in an in vitro binding assay, on the surface of an immune cell) of about or at least about 2-fold, 5-fold, 10-fold, 50-fold, 100-fold, 1000-fold or more, relative to the binding affinity of the wild-type IL-2 protein to the wild-type IL-2Rβ/γc complex.

For example, some IL-2 proteins comprise one or more amino acid substitutions selected from K35C, R38C, T41C, F42C, E61C, and V69C as defined by SEQ ID NO: 2. In some embodiments, the IL-2 protein forms a disulfide bond with the IL-2 binding protein (e.g., IL-2Ra) via one or more of the cysteine substitutions selected from K35C, R38C, T41C, F42C, E61C, and V69C. Certain IL-2 proteins comprise one or more amino acid substitutions at position V69, Q74, and/or 1128 as defined by SEQ ID NO: 2, including combinations thereof and including, for example, wherein the one or more amino acid substitutions are selected from V69A, Q74P, and I128T as defined by SEQ ID NO: 2. Some IL-2 proteins comprise one or more amino acid substitutions at position T3, D20, R38, F42, Y45, E61, E62, E68, and/or L72 as defined by SEQ ID NO: 2, including combinations thereof. Exemplary amino acid substitutions include T3A; D20T; R38A, R38E, and R38K; F42A, F42G, F42S, F42T, F42Q, F42E, F42N, F42D, F42R, F42K, and F421; Y45A, Y45G, Y45S, Y45T, Y45Q, Y45E, Y45N, Y45D, Y45R, and Y45K; E61S and E61K; E62A, E62L, and E62K; E68A and E68V; and L72A, L72G, L72S, L72T, L72Q, L72E, L72N, L72D, L72R, and L72K, including combinations thereof. Particular examples of combinations of substitutions include T3A and F42A; T3A, V69A, Q74P, and I128T; T3A and D20T; T3A, D20T, and F42A; T3A and R38E; T3A and E62K; T3A, E61K, and E62K; T3A, R38E, E61K, E62K; T3A, R38E, and F42A; T3A, F42A, and E61K; T3A, F42A, and E62K; and T3A, F42A, E61K, and E62K. Thus, an IL-2 protein can comprise any one or more of the foregoing amino acid substitutions, including combinations thereof.

In certain embodiments, a potential O-glycosylation site in IL-2 is substituted with alanine (T3A). In some embodiments, an F42A substitution in IL-2 reduces IL-2 binding affinity towards IL-2Rα. In some embodiments, a triple mutant (V69A, Q74P, and I128T) of IL-2 has higher binding affinity towards IL-2Rα. In certain embodiments, a D20T substitution in IL-2 does not significantly reduce binding affinity to IL-2Rα binding affinity but significantly reduces signaling activity towards intermediate affinity IL-2R receptors.

In some embodiments, the IL-2 protein comprises one or more amino acid substitutions at residues selected from A1, P2, A3, S4, and S5, as defined by SEQ ID NO: 2 or 3, or comprises N-terminal deletion of 1, 2, 3, 4, or 5 amino acids, as defined by SEQ ID NO: 2 or 3.

Any one or more of the foregoing IL-2 proteins can be combined with any of the binding moieties, IL-2Rβ proteins, and linkers described herein, to generate one or more fusion proteins or larger, multi-chain structures comprising the same.

Interleukin-7 (IL-7). In certain embodiments, the cytokine is IL-7, and the wild-type cognate receptor comprises the IL-7R/λc complex on the surface of an immune cell. IL-7 is a hematopoietic growth factor secreted by stromal cells in the bone marrow and thymus. It is important for proliferation during certain stages of B-cell maturation, and T cell and NK cell survival, development, and homeostasis.

IL-7 binds to the IL-7 receptor (IL-7Rα), a heterodimer composed of two subunits, an interleukin-7 receptor-α (CD127) and common-γ chain receptor (CD132). Receptor binding results in a cascade of signals important for T-cell development within the thymus and survival within the periphery, among other activities. IL-7 as an immunotherapy agent has been examined for treatment of various malignancies and during HIV infection. For example, administration of IL-7 in patients with cancer has been shown to transiently disrupt the homeostasis of both CD8+ and CD4+ T cells with a commensurate decrease in the percentage of CD4+CD25+Foxp3+T regulatory cells. Associated with antiretroviral therapy, IL-7 has been shown to decrease local and systemic inflammations in patients that had incomplete T-cell reconstitution. Exemplary human IL-7 protein sequences are provided in Table S2.

TABLE S2
Exemplary IL-7 Polypeptides

| | | SEQ ID |
|---|---|---|
| Name | Sequence | NO. |
| Human IL-7 | MFHVSFRYIFGLPPLILVLLPVASSDCDIEGKDGKQYESVLMVSIDQLL | |
| Full-length | DSMKEIGSNCLNNEFNFFKRHICDANKEGMFLFRAARKLRQFLKMNSTG | |
| | DFDLHLLKVSEGTTILLNCTGQVKGRKPAALGEAQPTKSLEENKSLKEQ | |
| | KKLNDLCFLKRLLQEIKTCWNKILMGTKEH | |
| Human IL-7 | DCDIEGKDGKQYESVLMVSIDQLLDSMKEIGSNCLNNEFNFFKRHICDA | |
| Mature | NKEGMFLFRAARKLRQFLKMNSTGDFDLHLLKVSEGTTILLNCTGQVKG | |
| | RKPAALGEAQPTKSLEENKSLKEQKKLNDLCFLKRLLQEIKTCWNKILM | |
| | GTKEH | |
| Human IL-7 | DCDIEGKDGKQYESVLMVSIDQLLDSMKEIGSNCLNNEFNFFKRHICDA | |
| Mature | NKEGMFLFRAARKLRQFLKMNSTGDFDLHLLKVSEGTTILLNCTGQVKG | |
| E106A | RKPAALGAAQPTKSLEENKSLKEQKKLNDLCFLKRLLQEIKTCWNKILM | |
| | GTKEH | |

Thus, in certain embodiments, an IL-7 protein comprises, consists, or consists essentially of an amino acid sequence selected from Table S2, or an active variant or fragment thereof that is at least 80, 85, 90, 95, 98, or 100% identical to an amino acid sequence selected from Table S2, and binds to and signals through the IL-7Rα/λc complex.

In some embodiments, an “active” IL-7 protein or fragment or variant is characterized, for example, by its ability to bind to an IL-7Rα (CD127) or common-γ chain receptor (CD132) chain present on the surface of an immune cell in vitro or in vivo, and stimulate downstream signaling activities. Exemplary downstream signaling activities include stimulating the differentiation of multipotent (pluripotent) hematopoietic stem cells into lymphoid progenitor cells, and stimulating the proliferation of cells in the lymphoid lineage, including B cells, T cells, and NK cells.

Certain IL-7 proteins comprise one or more amino acid substitutions relative to the exemplary amino acid sequences in Table S_. In certain embodiments, the IL-7 protein is a variant that has increased binding affinity to the wild-type IL-7Rα/λc complex (e.g., in an in vitro binding assay, on the surface of an immune cell) of about or at least about 2-fold, 5-fold, 10-fold, 50-fold, 100-fold, 1000-fold or more, relative to the binding affinity of the wild-type IL-7 protein to the wild-type IL-7Rα/λc complex. In certain embodiments, the IL-7 protein is a variant that has reduced binding affinity to the wild-type IL-7Rα/λc complex (e.g., in an in vitro binding assay, on the surface of an immune cell) of about or at least about 2-fold, 5-fold, 10-fold, 50-fold, 100-fold, 1000-fold or more, relative to the binding affinity of the wild-type IL-7 protein to the wild-type IL-7Rα/λc complex. In some embodiments, an IL-7 protein variant comprises or retains a substitution at E106, as defined by the mature IL-7 sequence, including an E106A substitution.

Any one or more of the foregoing IL-7 polypeptides can be combined with any of the binding moieties, IL-7Rα proteins, and linkers described herein, to generate one or more fusion proteins or larger, multi-chain structures comprising the same.

Interleukin-15 (IL-15). In certain embodiments, the cytokine is IL-15, and the wild-type cognate receptor comprises IL-15Rβ/λc on the surface of an immune cell. IL-15 is a pleiotropic cytokine that has been shown to induce and regulate a myriad of immune functions. For example, IL-15 is critical for lymphoid development, peripheral maintenance of innate immune cells, and immunological memory of T cells, mainly natural killer (NK) and CD8+ T cell populations. Like IL-2, IL-15 binds to and signals through a complex composed of IL-2/IL-15 receptor beta chain (CD122) and the common gamma chain (gamma-C, CD132).

IL-15 is a 14-15 kDA protein composed of a signal peptide (residues 1-29), a propeptide (residues 30-48), and an active mature protein (residues 49-162). Exemplary human IL-15 protein sequences are provided in Table S3.

TABLE S3
Exemplary IL-15 polypeptide sequences

| | | SEQ ID |
|---|---|---|
| Name | Sequence | NO: |
| Human IL-15 | MRISKPHLRSISIQCYLCLLLNSHFLTEAGIHVFILGCFSAGLPKTEANW | |
| FL precursor | VNVISDLKKIEDLIQSMHIDATLYTESDVHPSCKVTAMKCFLLELQVISL | |
| | ESGDASIHDTVENLIILANNSLSSNGNVTESGCKECEELEEKNIKEFLQS | |
| | FVHIVQMFINTS | |
| Human IL-15 | NWVNVISDLKKIEDLIQSMHIDATLYTESDVHPSCKVTAMKCFLLELQVI | |
| mature form | SLESGDASIHDTVENLIILANNSLSSNGNVTESGCKECEELEEKNIKEFL | |
| | QSFVHIVQMFINTS | |
| Human IL-15 | NWVNVISDLKKIEDLIQSMHIDATLYTESDVHPSCKVTAMKCFLLELQVI | |
| mature form | SLESGDASIHDTVENLIILANNSLSSNGNVTESGCKECEELEEKNIKEFL | |
| with S162A | QSFVHIVQMFINTA | |
| Human IL-15 | NWVNVISDLKKIEDLIQSMHIDATLYTESDVHPSCKVTAMKCFLLELQDI | |
| mature form | SLESGDASIHDTVENLIILANNSLSSNGNVTESGCKECEELEEKNIKEFL | |
| with V49D, | QSFVHIVQMFINTA | |
| S162A | | |
| Human IL-15 | NWVNVISDLKKIEDLIQSMHIDATLYTESDVHPSCKVTAMKCFLLELQVD | |
| mature form | SLESGDASIHDTVENLIILANNSLSSNGNVTESGCKECEELEEKNIKEFL | |
| with I50D, | QSFVHIVQMFINTA | |
| S162A | | |
| Human IL-15 | NWVNVISDLKKIEDLIQSMHIDATLYTESDVHPSCKVTAMKCFLLELQVI | |
| mature form | SLESGDASIHDTVENEIILANNSLSSNGNVTESGCKECEELEEKNIKEFL | |
| with L66E, | QSFVHIVQMFINTA | |
| S162A | | |
| Human IL-15 | NWVNVISNLKKIEDLIQSMHIDATLYTESDVHPSCKVTAMKCFLLELQVI | |
| mature form | SLESGDASIHDTVENLIILANNSLSSNGNVTESGCKECEELEEKNIKEFL | |
| with D8N, | QSFVHIVQMFINTA | |
| S162A | | |
| Human IL-15 | NWVNVISDLKKIEDLIQSMHIDATLYTESDVHPSCKVTAMKCFLLKLQVI | |
| mature form | SLESGDASIHDTVENLIILANNSLSSNGNVTESGCKECEELEEKNIKEFL | |
| with E46K, | QSFVHIVQMFINTA | |
| S162A | | |
| Human IL-15 | NWVNVISDLKKIEDLIQSMHIDATLYTESDVHPSCKVTAMKCFLLKLQVI | |
| mature form | SLKSGDASIHDTVENLIILANNSLSSNGNVTESGCKECEELEEKNIKEFL | |
| with E46K, | QSFVHIVQMFINTA | |
| E53K, S162A | | |
| Human IL-15 | NWVNVISDLKKIEDLIQSMHIKATLYTESDVHPSCKVTAMKCFLLKLQVI | |
| mature form | SLESGDASIHDTVENLIILANNSLSSNGNVTESGCKECEELEEKNIKEFL | |
| with D22K, | QSFVHIVQMFINTA | |
| E46K, S162A | | |
| Human IL-15 | NWVNVISDLKKIEDLIQSMHIKATLYTESDVHPSCKVTAMKCFLLKLQVI | |
| mature form | SLKSGDASIHDTVENLIILANNSLSSNGNVTESGCKECEELEEKNIKEFL | |
| with D22K, | QSFVHIVQMFINTA | |
| E46K, E53K, | | |
| S162A | | |

Thus, in certain embodiments, an IL-15 protein comprises, consists, or consists essentially of an amino acid sequence selected from Table S3, or an active variant or fragment thereof that is at least 80, 85, 90, 95, 98, or 100% identical to an amino acid sequence selected from Table S3, and binds to and signals through IL-15Rβ/γc and/or IL-15Rα/β/γc.

In particular embodiments, the IL-15 protein is a mature form of IL-15, or an active variant or fragment thereof, which comprises, consists, or consists essentially of an amino acid sequence that is at least 80, 85, 90, 95, 98, or 100% identical to amino acids 49-162 of SEQ ID NO: 27 (Human IL-15 FL precursor). Certain IL-15 polypeptides comprise, consist, or consist essentially of an amino acid sequence that is at least 80, 85, 90, 95, 98, or 100% identical to SEQ ID NO: 28 (mature human IL-15).

In some embodiments, an “active” IL-15 protein or fragment or variant is characterized, for example, by its ability to bind to an IL-15Rβ/γc and/or IL-15Rα/β/γc receptor chain present on the surface of an immune cell in vitro or in vivo, and stimulate downstream signaling activities. Examples of downstream signaling activities include IL-15 mediated signaling via Janus kinase 1 (Jak1) and γc subunit Janus kinase 3 (Jak3), which leads to phosphorylation and activation of signal transducer and activator of transcription 3 (STAT3) and STAT5 pathways. Additional examples include activation of Src family kinases including Lek and Fyn, and subsequent activation of PI3K and MAPK signaling pathways. Altogether, IL-15 signaling stimulates an array of downstream pathways leading to responses that have a significant role in the regulating the activation and proliferation of T and natural killer (NK) cells, and the survival of memory T cells, among others. In particular embodiments, an activated IL-15 polypeptide elicits potent antitumor responses upon activation in target tissues, that is, a tumor microenvironment.

Certain IL-15 polypeptides comprise one or more amino acid substitutions relative to the exemplary amino acid sequences in Table S3. In certain embodiments, the IL-15 protein is a variant that has increased binding affinity to the wild-type IL-15Rβ/γc complex (e.g., in an in vitro binding assay, on the surface of an immune cell) of about or at least about 2-fold, 5-fold, 10-fold, 50-fold, 100-fold, 1000-fold or more, relative to the binding affinity of the wild-type IL-15 protein to the wild-type IL-15Rβ/γc complex. In certain embodiments, the IL-15 protein is a variant that has reduced binding affinity to the wild-type IL-15Rβ/γc complex (e.g., in an in vitro binding assay, on the surface of an immune cell) of about or at least about 2-fold, 5-fold, 10-fold, 50-fold, 100-fold, 1000-fold or more, relative to the binding affinity of the wild-type IL-15 protein to the wild-type IL-15Rβ/γc complex.

In certain embodiments, an IL-15 protein comprises or retains one or more amino acid substitutions at position D8, D22, E46, V49, 150, L66, and/or S162 as defined by SEQ ID NO: 26 (mature human IL-15). Specific examples of substitutions are selected from one or more of D8N, D22K, E46K, V49D, I50D, L66E, and 162A, including combinations thereof (see Table S3). Exemplar combinations of substitutions are selected from V49D and S162A; I50D and S162A; L66E and S162A; D8N and S162A; V49D and S162A; E46K and S162A; E46K, E53K, and S162A; D22K, E46K, and S162A; and D22K, E46K, E53K, and S162A.

In some embodiments, a D8N substitution in IL-15 does not significantly reduce binding affinity to IL-15Rα significantly reduces or all but eliminates IL-15 signaling activity. In some embodiments a V49D substitution in IL-15 has significantly lower (e.g., about 13 fold lower) binding affinity to IL-15Rα and retains about or at least about 90-100% of IL-15 signaling activity. In some embodiments, an I50D substitution in IL-15 has significantly lower (e.g., about 100 fold lower) binding affinity to IL-15Rα and retains about 10% of IL-15 signaling activity. In some embodiments, a L66E substitution in IL-15 has significantly lower (e.g., about 15 fold lower) binding affinity to IL-15Rα and retains little to no IL-15 signaling activity.

Any one or more of the foregoing IL-15 proteins can be combined with any of the binding moieties, IL-15Rβ proteins, and linkers described herein, to generate one or more fusion proteins or larger, multi-chain structures comprising the same.

Interleukin-21 (IL-21). In certain embodiments, the cytokine is IL-21, and the wild-type cognate receptor comprises IL-21R on the surface of an immune cell. IL-21 has potent regulatory effects on cells of the immune system, including NK cells and cytotoxic T cells, for example, by inducing cell division/proliferation in its target cells. IL-21 is expressed in activated human CD4+ T cells and NK T cells, and is also up-regulated in Th2 and Th17 subsets of T helper cells and T follicular cells.

IL-21 binds to the IL-21 receptor (IL-21R), which is expressed on the surface of T cells, B cells, and NK cells. IL-21R is similar in structure to the receptors for other type I cytokines like IL-2R and IL-15R and requires dimerization with the common gamma chain (γc) in order to bind IL-21. When bound to IL-21, the IL-21R acts through the Jak/STAT pathway, utilizing Jak1 and Jak3 and a STAT3 homodimer to activate its target genes. IL-21 has shown utility in the treatment of cancers, viral infections, and a variety of inflammatory diseases. Exemplary human IL-21 amino acid sequences are provided in Table S4.

TABLE S4
Exemplary IL-21 Polypeptides

| | | SEQ ID |
|---|---|---|
| Name | Sequence | NO. |
| Human IL-21 | MRSSPGNMERIVICLMVIFLGTLVHKSSSQGQDRHMIRMRQLIDIVDQL | |
| Full-length | KNYVNDLVPEFLPAPEDVETNCEWSAFSCFQKAQLKSANTGNNERIINV | |
| | SIKKLKRKPPSTNAGRRQKHRLTCPSCDSYEKKPPKEFLERFKSLLQKM | |
| | IHQHLSSRTHGSEDS | |
| Human IL-21 | HKSSSQGQDRHMIRMRQLIDIVDQLKNYVNDLVPEFLPAPEDVETNCEW | |
| Mature | SAFSCFQKAQLKSANTGNNERIINVSIKKLKRKPPSTNAGRRQKHRLTC | |
| | PSCDSYEKKPPKEFLERFKSLLQKMIHQHLSSRTHGSEDS | |
| Human IL-21 | QGQDRHMIRMRQLIDIVDQLKNYVNDLVPEFLPAPEDVETNCEWSAFSC | |
| (30-162) | FQKAQLKSANTGNNERIINVSIKKLKRKPPSTNAGRRQKHRLTCPSCDS | |
| | YEKKPPKEFLERFKSLLQKMIHQHLSSRTHGSEDS | |

Thus, in certain embodiments, an IL-21 protein comprises, consists, or consists essentially of an amino acid sequence selected from Table S4, or an active variant or fragment thereof that is at least 80, 85, 90, 95, 98, or 100% identical to an amino acid sequence selected from Table S4, and binds to and signals through IL-21R/γc.

In some embodiments, an “active” IL-21 protein or fragment or variant is characterized, for example, by its ability to bind to the IL-21R/γc complex present on the surface of an immune cell in vitro or in vivo, and stimulate downstream signaling activities. Exemplary downstream signaling activities include inducing cell division/proliferation of NK cells and cytotoxic T cells.

Certain IL-21 proteins comprise one or more amino acid substitutions relative to the exemplary amino acid sequences in Table S4. In certain embodiments, the IL-21 protein is a variant that has increased binding affinity to the wild-type IL-21R/λc complex (e.g., in an in vitro binding assay, on the surface of an immune cell) of about or at least about 2-fold, 5-fold, 10-fold, 50-fold, 100-fold, 1000-fold or more, relative to the binding affinity of the wild-type IL-21 protein to the wild-type IL-21R/λc complex. In certain embodiments, the IL-21 protein is a variant that has reduced binding affinity to the wild-type IL-21R/λc complex (e.g., in an in vitro binding assay, on the surface of an immune cell) of about or at least about 2-fold, 5-fold, 10-fold, 50-fold, 100-fold, 1000-fold or more, relative to the binding affinity of the wild-type IL-21 protein to the wild-type IL-21R/λc complex.

Any one or more of the foregoing IL-21 polypeptides can be combined with any of the binding moieties, IL-21R proteins, and linkers described herein, to generate one or more fusion proteins or larger, multi-chain structures comprising the same.

Interferons (IFNs). In certain embodiments, the cytokine is an IFN protein. IFNs are a class of cytokines that modulate immune responses, including anti-viral immune responses, for example, by up-regulating major histocompatibility complex molecules (MHC I and MHC II) and increasing immunoproteasome activity. IFNs also suppress angiogenesis, for example, by down-regulating angiogenic stimuli from tumor cells and suppressing the proliferation of endothelial cells. In some embodiments, the IFN protein is a type I IFN protein, a type II IFN protein, or a type III IFN protein, and the wild-type cognate receptor comprises a corresponding IFN receptor (IFNR) on the surface of an immune cell.

Type I IFNs are a subgroup of IFNs that bind to the IFN-α/β receptor (IFNAR), which is composed of IFNAR1 and IFNAR2 chains. Thus, in certain embodiments, the cytokine is a type I IFN and the wild-type cognate receptor is the IFNAR on the surface of an immune cell. In some embodiments, the type I IFN protein is selected from interferon-α (e.g., IFNA1, IFNA2, IFNA4, IFNA5, IFNA6, IFNA7, IFNA8, IFNA10, IFNA13, IFNA14, IFNA16, IFNA17, IFNA21), interferon-β (e.g., IFNB1, IFNB3), interferon-ω (IFNW1), and interferon-κ (IFNK), including active variants and fragments thereof.

IFN-α proteins are produced mainly by plasmacytoid dendritic cells (pDCs), and are primarily involved in regulating innate immunity against viral infection. Recombinant IFN-α is used for the treatment of cancer, including hairy cell leukemia, malignant melanoma and AIDS-related Kaposi's sarcoma, venereal or genital warts caused by the Human Papilloma Virus, hepatitis B, and hepatitis C.

IFN-β proteins are produced largely by fibroblasts, and have antiviral activity that is mostly involved in innate immune responses. Examples of IFN-β proteins include IFN-β1 (IFNB1) and IFN-β3 (IFNB3). IFN-β1 is used to treat multiple sclerosis and reduces the relapse rate in that disease.

Interferon-ω (IFNW1) in humans includes four pseudogenes and one full gene that is expressed in leukocytes, and shares about 62% amino acid sequence homology and similar functions with IFN-α, and about 33% amino acid similarity with IFN-β. Similar to other IFNs, IFN-ω is produced by cells in response to viral infection, among other stimuli. IFNW1 has been demonstrated to have antiviral, anti-proliferation, and antitumor activities that are similar to those of IFN-α.

Interferon-κ (IFNK) is expressed in keratinocytes and imparts cellular protection against viral infections in a species-specific manner. It activates the interferon-stimulated response element signaling pathway, and directly modulates cytokine release from monocytes and dendritic cells.

Exemplary human type I IFN amino acid sequences are provided in Table S5 below.

TABLE S5
Type I Interferons

| | | SEQ ID |
|---|---|---|
| Name | Sequence | NO: |
| IFNA1 | | |
| Human IFN-α2 | MALTFALLVALLVLSCKSSCSVGCDLPQTHSLGSRRTLMLLAQMRKISLFSCL | |
| (IFNA2) | KDRHDFGFPQEEFGNQFQKAETIPVLHEMIQQIFNLFSTKDSSAAWDETLLDK | |
| FL precursor | FYTELYQQLNDLEACVIQGVGVTETPLMKEDSILAVRKYFQRITLYLKEKKYS | |
| | PCAWEVVRAEIMRSFSLSTNLQESLRSKE | |
| Human IFN-α2 | CDLPQTHSLGSRRTLMLLAQMRKISLFSCLKDRHDFGFPQEEFGNQFQKAETI | |
| (IFNA2) | PVLHEMIQQIFNLFSTKDSSAAWDETLLDKFYTELYQQLNDLEACVIQGVGVT | |
| Mature | ETPLMKEDSILAVRKYFQRITLYLKEKKYSPCAWEVVRAEIMRSFSLSTNLQE | |
| (24-188) | SLRSKE | |
| Human IFN-α2 | CDLPQTHSLGSRRTLMLLAQMRRISLFSCLKDRHDFGFPQEEFGNQFQKAETI | |
| (IFNA2) | PVLHEMIQQIFNLFSTKDSSAAWDETLLDKFYTELYQQLNDLEACVIQGVGVT | |
| Mature variant | ETPLMKEDSILAVRKYFQRITLYLKEKKYSPCAWEVVRAEIMRSFSLSTNLQE | |
| (24-188) | SLRSKE | |
| IFN alfacon- | CDLPQTHSLGNRRALILLAQMRRISPFSCLKDRHDFGFPQEEFDGNQFQKAQA | |
| 1 | ISVLHEMIQQTFNLFSTKDSSAAWDESLLEKFYTELYQQLNDLEACVIQEVGV | |
| | EETPLMNVDSILAVKKYFQRITLYLTEKKYSPCAWEVVRAEIMRSFSLSTNLQ | |
| | ERLRRKE | |
| IFN-α2B | CDLPQTHSLGSRRTLMLLAQMRRISLFSCLKDRHDFGFPQEEFGNQFQKAETI | |
| | PVLHEMIQQIFNLFSTKDSSAAWDETLLDKFYTELYQQLNDLEACVIQGVGVT | |
| | ETPLMNEDSILAVRKYFQRITLYLKEKKYSPCAWEVVRAEIMRSFSLSTNLQE | |
| | SLRSKE | |
| IFN-α2c | CDLPQTHSLGSRRTLMLLAQMRRISLFSCLKDRRDFGFPQEEFGNQFQKAETI | |
| | PVLHEMIQQIFNLFSTKDSSAAWDETLLDKFYTELYQQLNDLEACVIQGVGVT | |
| | ETPLMNEDSILAVRKYFQRITLYLKEKKYSPCAWEVVRAEIMRSFSLSTNLQE | |
| | SLRSKE | |
| IFNA4 mature | CDLPQTHSLGNRRALILLAQMGRISHFSCLKDRHDFGFPEEEFDGHQFQKAQA | |
| | ISVLHEMIQQTFNLFSTEDSSAAWEQSLLEKFSTELYQQLNDLEACVIQEVGV | |
| | EETPLMNEDSILAVRKYFQRITLYLTEKKYSPCAWEVVRAEIMRSLSFSTNLQ | |
| | KRLRRKD | |
| IFNA5 mature | LGCDLPQTHSLSNRRTLMIMAQMGRISPFSCLKDRHDFGFPQEEFDGNQFQKA | |
| | QAISVLHEMIQQTFNLFSTKDSSATWDETLLDKFYTELYQQLNDLEACMMQEV | |
| | GVEDTPLMNVDSILTVRKYFQRITLYLTEKKYSPCAWEVVRAEIMRSFSLSAN | |
| | LQERLRRKE | |
| IFNA6 mature | SLDCDLPQTHSLGHRRTMMLLAQMRRISLFSCLKDRHDFRFPQEEFDGNQFQK | |
| | AEAISVLHEVIQQTFNLFSTKDSSVAWDERLLDKLYTELYQQLNDLEACVMQE | |
| | VWVGGTPLMNEDSILAVRKYFQRITLYLTEKKYSPCAWEVVRAEIMRSFSSSR | |
| | NLQERLRRKE | |
| IFNA7 mature | CDLPQTHSLRNRRALILLAQMGRISPFSCLKDRHEFRFPEEEFDGHQFQKTQA | |
| | ISVLHEMIQQTFNLFSTEDSSAAWEQSLLEKFSTELYQQLNDLEACVIQEVGV | |
| | EETPLMNEDFILAVRKYFQRITLYLMEKKYSPCAWEVVRAEIMRSFSFSTNLK | |
| | KGLRRKD | |
| IFNA8 mature | CDLPQTHSLGNRRALILLAQMRRISPFSCLKDRHDFEFPQEEFDDKQFQKAQA | |
| | ISVLHEMIQQTFNLFSTKDSSAALDETLLDEFYIELDQQLNDLESCVMQEVGV | |
| | IESPLMYEDSILAVRKYFQRITLYLTEKKYSSCAWEVVRAEIMRSFSLSINLQ | |
| | KRLKSKE | |
| IFNA10 | CDLPQTHSLGNRRALILLGQMGRISPFSCLKDRHDFRIPQEEFDGNQFQKAQA | |
| mature | ISVLHEMIQQTFNLFSTEDSSAAWEQSLLEKFSTELYQQLNDLEACVIQEVGV | |
| | EETPLMNEDSILAVRKYFQRITLYLIERKYSPCAWEVVRAEIMRSLSFSTNLQ | |
| | KRLRRKD | |
| IFNA1/13 | CDLPETHSLDNRRTLMLLAQMSRISPSSCLMDRHDFGFPQEEFDGNQFQKAPA | |
| mature | ISVLHELIQQIFNLFTTKDSSAAWDEDLLDKFCTELYQQLNDLEACVMQEERV | |
| | GETPLMNADSILAVKKYFRRITLYLTEKKYSPCAWEVVRAEIMRSLSLSTNLQ | |
| | ERLRRKE | |
| IFNA14 | CNLSQTHSLNNRRTLMLMAQMRRISPFSCLKDRHDFEFPQEEFDGNQFQKAQA | |
| mature | ISVLHEMMQQTFNLFSTKNSSAAWDETLLEKFYIELFQQMNDLEACVIQEVGV | |
| | EETPLMNEDSILAVKKYFQRITLYLMEKKYSPCAWEVVRAEIMRSLSFSTNLQ | |
| | KRLRRKD | |
| IFNA16 | CDLPQTHSLGNRRALILLAQMGRISHFSCLKDRYDFGFPQEVFDGNQFQKAQA | |
| mature | ISAFHEMIQQTFNLFSTKDSSAAWDETLLDKFYIELFQQLNDLEACVTQEVGV | |
| | EEIALMNEDSILAVRKYFQRITLYLMGKKYSPCAWEVVRAEIMRSFSFSTNLQ | |
| | KGLRRKD | |
| IFNA17 | CDLPQTHSLGNRRALILLAQMGRISPFSCLKDRHDFGLPQEEFDGNQFQKTQA | |
| mature | ISVLHEMIQQTFNLFSTEDSSAAWEQSLLEKFSTELYQQLNNLEACVIQEVGM | |
| | EETPLMNEDSILAVRKYFQRITLYLTEKKYSPCAWEVVRAEIMRSLSFSTNLQ | |
| | KILRRKD | |
| IFNA21 | CDLPQTHSLGNRRALILLAQMGRISPFSCLKDRHDFGFPQEEFDGNQFQKAQA | |
| mature | ISVLHEMIQQTFNLFSTKDSSATWEQSLLEKFSTELNQQLNDLEACVIQEVGV | |
| | EETPLMNVDSILAVKKYFQRITLYLTEKKYSPCAWEVVRAEIMRSFSLSKIFQ | |
| | ERLRRKE | |
| IFNB1 mature | MSYNLLGFLQRSSNFQCQKLLWQLNGRLEYCLKDRMNFDIPEEIKQLQQFQKE | |
| | DAALTIYEMLQNIFAIFRQDSSSTGWNETIVENLLANVYHQINHLKTVLEEKL | |
| | EKEDFTRGKLMSSLHLKRYYGRILHYLKAKEYSHCAWTIVRVEILRNFYFINR | |
| | LTGYLRN | |
| IFNB3 | | |
| IFNW1 mature | CDLPQNHGLLSRNTLVLLHQMRRISPFLCLKDRRDFRFPQEMVKGSQLQKAHV | |
| | MSVLHEMLQQIFSLFHTERSSAAWNMTLLDQLHTGLHQQLQHLETCLLQVVGE | |
| | GESAGAISSPALTLRRYFQGIRVYLKEKKYSDCAWEVVRMEIMKSLFLSTNMQ | |
| | ERLRSKDRDLGSS | |
| IFNK mature | LDCNLLNVHLRRVTWQNLRHLSSMSNSFPVECLRENIAFELPQEFLQYTQPMK | |
| | RDIKKAFYEMSLQAFNIFSQHTFKYWKERHLKQIQIGLDQQAEYLNQCLEEDK | |
| | NENEDMKEMKENEMKPSEARVPQLSSLELRRYFHRIDNFLKEKKYSDCAWEIV | |
| | RVEIRRCLYYFYKFTALFRRK | |

Thus, in certain embodiments, a cytokine is a type I IFN protein that comprises, consists, or consists essentially of an amino acid sequence selected from Table S5, or an active variant or fragment thereof that is at least 80, 85, 90, 95, 98, or 100% identical to an amino acid sequence selected from Table S5, and binds to and signals through IFNAR.

In some embodiments, an “active” type I IFN protein, or variant of fragment thereof, is characterized, for example, by its ability to bind to IFNAR on the surface of an immune cell in vitro or in vivo, and stimulate downstream signaling activities, absent steric hindrance by the binding moieties described herein. Certain exemplary downstream activities include upregulating the expression of MHC I proteins, which increases presentation of peptides derived from viral antigens and thereby enhances the activation of CD8+ cytotoxic T cells, inducing antiviral mediators such as 2′-5′ oligoadenylate synthetase (2′-5′ A synthetase) and protein kinase R, and activating Jak (Janus kinase) tyrosine kinases (Jak1 and Tyk2) and Stat1 and Stat2 (signal transducers and activators of transcription). In some embodiments, an IFN-α polypeptide has anti-viral and/or anti-cancer activities.

Certain type I IFN polypeptides comprise one or more amino acid substitutions relative to the exemplary amino acid sequences in Table S5. In certain embodiments, the type I IFN protein is a variant that has increased binding affinity to wild-type IFNAR (e.g., in an in vitro binding assay, on the surface of an immune cell) of about or at least about 2-fold, 5-fold, 10-fold, 50-fold, 100-fold, 1000-fold or more, relative to the binding affinity of the corresponding wild-type type I IFN protein to wild-type IFNAR. In certain embodiments, the type I IFN protein is a variant that has reduced binding affinity to wild-type IFNAR (e.g., in an in vitro binding assay, on the surface of an immune cell) of about or at least about 2-fold, 5-fold, 10-fold, 50-fold, 100-fold, 1000-fold or more, relative to the binding affinity of the corresponding wild-type type I IFN protein to wild-type IFNAR. In some embodiments, the type I IFN protein is a human IFNA2 variant that comprises or retains a K23 substitution, as defined by the mature IFNA2 sequence, including a K23R substitution.

Type II IFNs have only one member, interferon-γ (IFN-γ), which is an anti-parallel homodimer that binds to the IFN-γ receptor (IFNGR) complex. Generally, IFN-γ regulates immune and inflammatory responses, and is produced, for example, in activated T-cells and natural killer cells. The IFNGR complex is composed of two ligand-binding IFNGR1 chains linked to two signal-transducing IFNGR2 chains, and the associated signaling machinery. The intracellular domain of the IFNGR1 chain contains binding motifs for Jak1 and Stat1, and the IFNGR2 is an intracellular region that has a noncontiguous binding motif for recruitment of Jak2 kinase for signaling.

Thus, in certain embodiments, the type II IFN protein is IFN-γ, for example, the IFNG1 subunit, the IFNG2 subunit, or both subunits. Exemplary human type II IFN amino acid sequences are provided in Table S6 below.

TABLE S6
Type II IFNs

| | | SEQ ID |
|---|---|---|
| Name | Sequence | NO: |
| IFN-γ | MKYTSYILAFQLCIVLGSLGCYCQDPYVKEAENLKKYFNAGHSDVADNGTLFLGILKN | |
| FL | WKEESDRKIMQSQIVSFYFKLFKNFKDDQSIQKSVETIKEDMNVKFFNSNKKKRDDFE | |
| | KLTNYSVTDLNVQRKAIHELIQVMAELSPAAKTGKRKRSQMLFRGRRASQ | |
| IFN-γ | QDPYVKEAENLKKYFNAGHSDVADNGTLFLGILKNWKEESDRKIMQSQIVSFYFKLFK | |
| mature | NFKDDQSIQKSVETIKEDMNVKFFNSNKKKRDDFEKLTNYSVTDLNVQRKAIHELIQV | |
| | MAELSPAAKTGKRKRSQMLFRG | |

Thus, in certain embodiments, a cytokine is a type II IFN protein that comprises, consists, or consists essentially of an amino acid sequence selected from Table S6, or an active variant or fragment thereof that is at least 80, 85, 90, 95, 98, or 100% identical to an amino acid sequence selected from Table S6, and binds to and signals through the IFNGR complex.

In some embodiments, an “active” type II IFN protein, or variant of fragment thereof, is characterized, for example, by its ability to bind to the IFNGR complex on the surface of an immune cell in vitro or in vivo, and stimulate downstream signaling activities, absent steric hindrance by the binding moieties described herein. Certain exemplary downstream activities include activating macrophages (e.g., by increasing antigen presentation and lysosome activity), reducing the proliferation of tumor cells (e.g., by inducing apoptosis or autophagy), inducing increased expression of MHC molecules, promoting leukocyte migration (e.g., via promoting adhesion and binding), inducing expression of intrinsic defense factors (e.g., TRIM5α, APOBEC, tetherin), inducing the production of IgG2a and IgG3 from activated plasma B cells, and potentiating the anti-viral and anti-tumor activities of type I interferons.

Certain type II IFN polypeptides comprise one or more amino acid substitutions relative to the exemplary amino acid sequences in Table S6. In certain embodiments, the type II IFN protein is a variant that has increased binding affinity to the wild-type IFNGR complex (e.g., in an in vitro binding assay, on the surface of an immune cell) of about or at least about 2-fold, 5-fold, 10-fold, 50-fold, 100-fold, 1000-fold or more, relative to the binding affinity of the corresponding wild-type type II IFN protein to the wild-type IFNGR complex. In certain embodiments, the type II IFN protein is a variant that has reduced binding affinity to the wild-type IFNGR complex (e.g., in an in vitro binding assay, on the surface of an immune cell) of about or at least about 2-fold, 5-fold, 10-fold, 50-fold, 100-fold, 1000-fold or more, relative to the binding affinity of the corresponding wild-type type II IFN protein to the wild-type IFNGR complex.

Type III IFNs refer to a group that consists of four IFN-λ (lambda) molecules. In certain embodiments, the type III IFN protein is IFN-λ, for example, which is selected from one or more of IFN-λ1, IFN-λ2, IFN-λ3, and IFN-λ4. Type III IFNs bind to and signal through a receptor complex consisting of IL10Rβ and IFN-λR1 (IFNLR1), the latter being largely expressed in tissues of epithelial origin. These lambda interferons are mainly involved in immune responses to viral infections and tumors, and signaling initiated by IFN-λ triggers the JAK-STAT pathway, leading to the expression of numerous interferon-stimulated genes with anti-viral and anti-proliferative effects. Exemplary human type III IFN amino acid sequences are provided in Table S7 below.

TABLE S7
Type III IFNs

| | | SEQ ID |
|---|---|---|
| Name | Sequence | NO: |
| IFN-λ1 | GPVPTSKPTTTGKGCHIGRFKSLSPQELASFKKARDALEESLKLKNWSCSSPVFPGNW | |
| mature | DLRLLQVRERPVALEAELALTLKVLEAAAGPALEDVLDQPLHTLHHILSQLQACIQPQ | |
| | PTAGPRPRGRLHHWLHRLQEAPKKESAGCLEASVTFNLFRLLTRDLKYVADGNLCLRT | |
| | STHPEST | |
| IFN-λ2 | VPVARLHGALPDARGCHIAQFKSLSPQELQAFKRAKDALEESLLLKDCRCHSRLFPRT | |
| mature | WDLRQLQVRERPMALEAELALTLKVLEATADTDPALVDVLDQPLHTLHHILSQFRACI | |
| | QPQPTAGPRTRGRLHHWLYRLQEAPKKESPGCLEASVTFNLFRLLTRDLNCVASGDLC | |
| | V | |
| IFN-λ3 | VPVARLRGALPDARGCHIAQFKSLSPQELQAFKRAKDALEESLLLKDCKCRSRLFPRT | |
| mature | WDLRQLQVRERPVALEAELALTLKVLEATADTDPALGDVLDQPLHTLHHILSQLRACI | |
| | QPQPTAGPRTRGRLHHWLHRLQEAPKKESPGCLEASVTFNLFRLLTRDLNCVASGDLC | |
| | V | |
| IFN-λ4 | AAPRRCELSHYRSLEPRTLAAAKALRDRYEEEALSWGQRNCSFRPRRDPPRPSSCARE | |
| mature | RHVARGIADAQAVLSGLHRSELLPGAGPILELLAAAGRDVAACLELARPGSSRKVPGA | |
| | QKRRHKPRRADSPRCRKASVVFNLLRLLTWELRLAAHSGPCL | |

Thus, in certain embodiments, a cytokine is a type III IFN protein that comprises, consists, or consists essentially of an amino acid sequence selected from Table S7, or an active variant or fragment thereof that is at least 80, 85, 90, 95, 98, or 100% identical to an amino acid sequence selected from Table S7, and binds to and signals through the IL10Rβ/IFNLR1 complex.

In some embodiments, an “active” type II IFN protein, or variant of fragment thereof, is characterized, for example, by its ability to bind to the IFNGR complex on the surface of an immune cell in vitro or in vivo, and stimulate downstream signaling activities, absent steric hindrance by the binding moieties described herein. Certain exemplary downstream activities include activating the JAK-STAT pathway, up-regulating certain interferon-stimulated genes (ISGs), and up-regulating MHC class I antigen expression.

Certain type III IFN polypeptides comprise one or more amino acid substitutions relative to the exemplary amino acid sequences in Table S7. In certain embodiments, the type III IFN protein is a variant that has increased binding affinity to the wild-type IL10Rβ/IFNLR1 complex (e.g., in an in vitro binding assay, on the surface of an immune cell) of about or at least about 2-fold, 5-fold, 10-fold, 50-fold, 100-fold, 1000-fold or more, relative to the binding affinity of the corresponding wild-type type III IFN protein to the wild-type IL10Rβ/IFNLR1 complex. In certain embodiments, the type III IFN protein is a variant that has reduced binding affinity to the wild-type IL10Rβ/IFNLR1 complex (e.g., in an in vitro binding assay, on the surface of an immune cell) of about or at least about 2-fold, 5-fold, 10-fold, 50-fold, 100-fold, 1000-fold or more, relative to the binding affinity of the corresponding wild-type type III IFN protein to the wild-type IL10Rβ/IFNLR1 complex.

Any of the foregoing IFN proteins can be combined with any of the binding moieties, corresponding IFNRs, and linkers described herein, to generate one or more activatable proprotein or procytokine homodimers, or larger, multi-chain structures comprising the same.

Cytokine Receptors. The activatable proprotein homodimers described herein comprise at least one cytokine receptor or cytokine receptor protein, including wild-type cytokine receptors and active variants and fragments thereof. In certain embodiments, the cytokine receptor comprises a human cytokine receptor sequence. Typically, the cytokine receptor is the natural receptor to the cytokine, for example, if the cytokine is IL-2 the cytokine receptor is IL-2Rβ; if the cytokine is IL-7, the cytokine receptor is IL-7Rα; if the cytokine is IL-15, the cytokine receptor is IL-15Rβ; if the cytokine is IL-21, the cytokine receptor is IL-21R; if the cytokine is a type I IFN, the cytokine receptor is IFNAR2; if the cytokine is a type II IFN, the cytokine receptor is IFNGR1; or if the cytokine is a type III IFN, the cytokine receptor is IL10Rβ or IFN-λR1.

In some embodiments, the cytokine receptor is a variant that comprises one or more amino acid alterations relative to the corresponding wild-type cytokine receptor, and has reduced binding affinity to the cytokine (in the proprotein or procytokine) relative to that of the wild-type cytokine receptor. For example, in some embodiments, the cytokine receptor variant has reduced binding affinity to the cytokine of about or at least about 2-fold, 5-fold, 10-fold, 50-fold, 100-fold, 1000-fold or more, relative to the binding affinity of the wild-type cytokine receptor to the cytokine. In some embodiments, the cytokine receptor comprises, consists, or consists essentially of the extracellular domain (ECD) of the cytokine receptor. Individual cytokine receptors are described in further detail herein.

Interleukin-2 Receptor β (IL-2Rβ). In certain embodiments, if the cytokine is an IL-2 protein, the cytokine receptor is an IL-2Rβ protein (or CD122), or a variant or fragment (e.g., extracellular domain or ECD) thereof. Exemplary human IL-2Rβ amino acid sequences are provided in Table S8 below.

TABLE S8
Exemplary IL-2Rβ Sequences

| | | SEQ ID |
|---|---|---|
| Name | Sequence | NO: |
| IL-2Rβ FL | MAAPALSWRLPLLILLLPLATSWASAAVNGTSQFTCFYNSRAN1SCVWSQDGALQ | |
| | DTSCQVHAWPDRRRWNQTCELLPVSQASWACNLILGAPDSQKLTTVDIVTLRVLC | |
| | REGVRWRVMAIQDFKPFENLRLMAPISLQVVHVETHRCNISWEISQASHYFERHL | |
| | EFEARTLSPGHTWEEAPLLTLKQKQEWICLETLTPDTQYEFQVRVKPLQGEFTTW | |
| | SPWSQPLAFRTKPAALGKDTIPWLGHLLVGLSGAFGFIILVYLLINCRNTGPWLK | |
| | KVLKCNTPDPSKFFSQLSSEHGGDVQKWLSSPFPSSSFSPGGLAPEISPLEVLER | |
| | DKVTQLLLQQDKVPEPASLSSNHSLTSCFTNQGYFFFHLPDALEIEACQVYFTYD | |
| | PYSEEDPDEGVAGAPTGSSPQPLQPLSGEDDAYCTFPSRDDLLLFSPSLLGGPSP | |
| | PSTAPGGSGAGEERMPPSLQERVPRDWDPQPLGPPTPGVPDLVDFQPPPELVLRE | |
| | AGEEVPDAGPREGVSFPWSRPPGQGEFRALNARLPLNTDAYLSLQELQGQDPTHL | |
| | V | |
| IL-2Rβ w/o | AVNGTSQFTCFYNSRANISCVWSQDGALQDTSCQVHAWPDRRRWNQTCELLPVSQ | |
| signal | ASWACNLILGAPDSQKLTTVDIVTLRVLCREGVRWRVMAIQDFKPFENLRLMAPI | |
| peptide | SLQVVHVETHRCNISWEISQASHYFERHLEFEARTLSPGHTWEEAPLLTLKQKQE | |
| (27-551) | WICLETLTPDTQYEFQVRVKPLQGEFTTWSPWSQPLAFRTKPAALGKDTIPWLGH | |
| | LLVGLSGAFGFIILVYLLINCRNTGPWLKKVLKCNTPDPSKFFSQLSSEHGGDVQ | |
| | KWLSSPFPSSSFSPGGLAPEISPLEVLERDKVTQLLLQQDKVPEPASLSSNHSLT | |
| | SCFTNQGYFFFHLPDALEIEACQVYFTYDPYSEEDPDEGVAGAPTGSSPQPLQPL | |
| | SGEDDAYCTFPSRDDLLLFSPSLLGGPSPPSTAPGGSGAGEERMPPSLQERVPRD | |
| | WDPQPLGPPTPGVPDLVDFQPPPELVLREAGEEVPDAGPREGVSFPWSRPPGQGE | |
| | FRALNARLPLNTDAYLSLQELQGQDPTHLV | |
| IL-2Rβ ECD | AVNGTSQFTCFYNSRANISCVWSQDGALQDTSCQVHAWPDRRRWNQTCELLPVSQ | |
| (27-240) | ASWACNLILGAPDSQKLTTVDIVTLRVLCREGVRWRVMAIQDFKPFENLRLMAPI | |
| | SLQVVHVETHRCNISWEISQASHYFERHLEFEARTLSPGHTWEEAPLLTLKQKQE | |
| | WICLETLTPDTQYEFQVRVKPLQGEFTTWSPWSQPLAFRTKPAALGKDT | |

Thus, in certain embodiments, the cytokine receptor is an IL-2Rβ3 protein that comprises, consists, or consists essentially of an amino acid sequence selected from Table S8, or an active variant or fragment thereof that is at least 80, 85, 90, 95, 98, or 1000% identical to an amino acid sequence selected from Table S8, and which binds to an IL-2 portion of the activatable proprotein homodimer.

In certain embodiments, the IL-2Rβ3 protein is a variant that comprises one or more amino acid alterations relative to wild-type IL-2Rβ, and which has reduced binding affinity to IL-2 relative to that of wild-type IL-2Rβ. For instance, in some embodiments, the IL-2Rβ protein variant has reduced binding affinity to IL-2 of about or at least about 2-fold, 5-fold, 10-fold, 50-fold, 100-fold, 1000-fold or more, relative to the binding affinity of IL-2Rβ to IL-2.

Any of the foregoing IL-2Rβ proteins can be combined with any of the binding moieties, corresponding IL-2 proteins, and linkers described herein, to generate one or more activatable proprotein or procytokine homodimers, or larger, multi-chain structures comprising the same.

Interleukin-7 Receptor (IL-7Rα). In certain embodiments, if the cytokine is an IL-7 protein, the cytokine receptor is an IL-7Rα protein (or CD127), or a variant or fragment (e.g., extracellular domain or ECD) thereof. Exemplary human IL-7Rα amino acid sequences are provided in Table S9 below.

TABLE S9
Exemplary IL-7Rα Sequences

| | | SEQ ID |
|---|---|---|
| Name | Sequence | NO: |
| IL-7Rα FL | MTILGTTFGMVFSLLQVVSGESGYAQNGDLEDAELDDYSFSCYSQLEVNGSQHS | |
| | LTCAFEDPDVNITNLEFEICGALVEVKCLNFRKLQEIYFIETKKFLLIGKSNIC | |
| | VKVGEKSLTCKKIDLTTIVKPEAPFDLSVVYREGANDFVVTFNTSHLQKKYVKV | |
| | LMHDVAYRQEKDENKWTHVNLSSTKLTLLQRKLQPAAMYEIKVRSIPDHYFKGF | |
| | WSEWSPSYYFRTPEINNSSGEMDPILLTISILSFFSVALLVILACVLWKKRIKP | |
| | IVWPSLPDHKKTLEHLCKKPRKNLNVSFNPESFLDCQIHRVDDIQARDEVEGFL | |
| | QDTFPQQLEESEKQRLGGDVQSPNCPSEDVVITPESFGRDSSLTCLAGNVSACD | |
| | APILSSSRSLDCRESGKNGPHVYQDLLLSLGTTNSTLPPPFSLQSGILTLNPVA | |
| | QGQPILTSLGSNQEEAYVTMSSFYQNQ | |
| IL-7Rα w/o | ESGYAQNGDLEDAELDDYSFSCYSQLEVNGSQHSLTCAFEDPDVNITNLEFEIC | |
| signal | GALVEVKCLNFRKLQEIYFIETKKFLLIGKSNICVKVGEKSLTCKKIDLTTIVK | |
| peptide | PEAPFDLSVVYREGANDFVVTFNTSHLQKKYVKVLMHDVAYRQEKDENKWTHVN | |
| (21-459) | LSSTKLTLLQRKLQPAAMYEIKVRSIPDHYFKGFWSEWSPSYYFRTPEINNSSG | |
| | EMDPILLTISILSFFSVALLVILACVLWKKRIKPIVWPSLPDHKKTLEHLCKKP | |
| | RKNLNVSFNPESFLDCQIHRVDDIQARDEVEGFLQDTFPQQLEESEKQRLGGDV | |
| | QSPNCPSEDVVITPESFGRDSSLTCLAGNVSACDAPILSSSRSLDCRESGKNGP | |
| | HVYQDLLLSLGTTNSTLPPPFSLQSGILTLNPVAQGQPILTSLGSNQEEAYVTM | |
| | SSFYQNQ | |
| IL-7Rα ECD | ESGYAQNGDLEDAELDDYSFSCYSQLEVNGSQHSLTCAFEDPDVNITNLEFEIC | |
| (21-239) | GALVEVKCLNFRKLQEIYFIETKKFLLIGKSNICVKVGEKSLTCKKIDLTTIVK | |
| | PEAPFDLSVVYREGANDFVVTFNTSHLQKKYVKVLMHDVAYRQEKDENKWTHVN | |
| | LSSTKLTLLQRKLQPAAMYEIKVRSIPDHYFKGFWSEWSPSYYFRTPEINNSSG | |
| | EMD | |
| IL-7Rα ECD | ESGYAQNGDLEDAELDDYSFSCYSQLEVNGSQHSLTCAFEDPDVNTTNLEFEIC | |
| variant | GALVEVKCLNFRKLQEIYFIETKKFLLIGKSNICVKVGEKSLTCKKIDLTTIVK | |
| (21-239) | PEAPFDLSVVYREGANDFVVTFNTSHLQKKYVKVLMHDVAYRQEKDENKWTHVN | |
| | LSSTKLTLLQRKLQPAAMYEIKVRSIPDHYFKGFWSEWSPSYYFRTPEINNSSG | |
| | EMD | |
| IL-7Rα ECD | ESGYAQNGDLEDAELDDYSFSCYSQLEVNGSQHSLTCAFEDPDVNTTNLEFEIC | |
| variant | GALVEVKCLNFRKLQEIYFIETAKFLLIGKSNICVKVGEKSLTCKKIDLTTIVK | |
| (21-239) | PEAPFDLSVVYREGANDFVVTFNTSHLQKKYVKVLMHDVAYRQEKDENKWTHVN | |
| | LSSTKLTLLQRKLQPAAMYEIKVRSIPDHYFKGFWSEWSPSYYFRTPEINNSSG | |
| | EMD | |
| IL-7Rα ECD | ESGYAQNGDLEDAELDDYSFSCYSQLEVNGSQHSLTCAFEDPDVNTTNLEFEIC | |
| variant | GALVEVKCLNFRKLQEIYFIETKKFALIGKSNICVKVGEKSLTCKKIDLTTIVK | |
| (21-239) | PEAPFDLSVVYREGANDFVVTFNTSHLQKKYVKVLMHDVAYRQEKDENKWTHVN | |
| | LSSTKLTLLQRKLQPAAMYEIKVRSIPDHYFKGFWSEWSPSYYFRTPEINNSSG | |
| | EMD | |

Thus, in certain embodiments, the cytokine receptor is an IL-7Rα protein that comprises, consists, or consists essentially of an amino acid sequence selected from Table S9, or an active variant or fragment thereof that is at least 80, 85, 90, 95, 98, or 1000% identical to an amino acid sequence selected from Table S9, and which binds to an IL-7 portion of the activatable proprotein homodimer.

In certain embodiments, the IL-7Rα protein is a variant that comprises one or more amino acid alterations relative to wild-type IL-7Rα, and which has reduced binding affinity to IL-7 relative to that of wild-type IL-7Rα. For instance, in some embodiments, the IL-7Rα protein variant has reduced binding affinity to IL-7 of about or at least about 2-fold, 5-fold, 10-fold, 50-fold, 100-fold, 1000-fold or more, relative to the binding affinity of IL-7Rα to IL-7. For example, in certain embodiments, an IL-7Rα protein variant comprises or retains a substitution at any one or more of I43, K77, and/or L80, as defined by the mature IL-7Rα sequence, including any one or more an 46T substitution, a K77A substitution, and/or an L80A substitution.

Any of the foregoing IL-7Rα proteins can be combined with any of the binding moieties, corresponding IL-7 proteins, and linkers described herein, to generate one or more activatable proprotein or procytokine homodimers, or larger, multi-chain structures comprising the same.

Interleukin-15 Receptor β (IL-15Rβ). In certain embodiments, if the cytokine is an IL-15 protein, the cytokine receptor is an IL-15Rβ protein (also CD122 or IL-2Rβ; IL-2 and IL-15 share a common beta receptor chain), or a variant or fragment (e.g., extracellular domain or ECD) thereof. Exemplary human IL-15Rβ) amino acid sequences are provided in Table S10 below.

TABLE S10
Exemplary IL-15Rβ Sequences

| | | SEQ ID |
|---|---|---|
| Name | Sequence | NO: |
| IL-15Rβ | MAAPALSWRLPLLILLLPLATSWASAAVNGTSQFTCFYNSRANISCVWSQDGALQD | |
| FL | TSCQVHAWPDRRRWNQTCELLPVSQASWACNLILGAPDSQKLTTVDIVTLRVLCRE | |
| | GVRWRVMAIQDFKPFENLRLMAPISLQVVHVETHRCNISWEISQASHYFERHLEFE | |
| | ARTLSPGHTWEEAPLLTLKQKQEWICLETLTPDTQYEFQVRVKPLQGEFTTWSPWS | |
| | QPLAFRTKPAALGKDTIPWLGHLLVGLSGAFGFIILVYLLINCRNTGPWLKKVLKC | |
| | NTPDPSKFFSQLSSEHGGDVQKWLSSPFPSSSFSPGGLAPEISPLEVLERDKVTQL | |
| | LLQQDKVPEPASLSSNHSLTSCFTNQGYFFFHLPDALEIEACQVYFTYDPYSEEDP | |
| | DEGVAGAPTGSSPQPLQPLSGEDDAYCTFPSRDDLLLFSPSLLGGPSPPSTAPGGS | |
| | GAGEERMPPSLQERVPRDWDPQPLGPPTPGVPDLVDFQPPPELVLREAGEEVPDAG | |
| | PREGVSFPWSRPPGQGEFRALNARLPLNTDAYLSLQELQGQDPTHLV | |
| IL-15Rβ | AVNGTSQFTCFYNSRANISCVWSQDGALQDTSCQVHAWPDRRRWNQTCELLPVSQA | |
| w/o | SWACNLILGAPDSQKLTTVDIVTLRVLCREGVRWRVMAIQDFKPFENLRLMAPISL | |
| signal | QVVHVETHRCNISWEISQASHYFERHLEFEARTLSPGHTWEEAPLLTLKQKQEWIC | |
| peptide | LETLTPDTQYEFQVRVKPLQGEFTTWSPWSQPLAFRTKPAALGKDTIPWLGHLLVG | |
| (27-551) | LSGAFGFIILVYLLINCRNTGPWLKKVLKCNTPDPSKFFSQLSSEHGGDVQKWLSS | |
| | PFPSSSFSPGGLAPEISPLEVLERDKVTQLLLQQDKVPEPASLSSNHSLTSCFTNQ | |
| | GYFFFHLPDALEIEACQVYFTYDPYSEEDPDEGVAGAPTGSSPQPLQPLSGEDDAY | |
| | CTFPSRDDLLLFSPSLLGGPSPPSTAPGGSGAGEERMPPSLQERVPRDWDPQPLGP | |
| | PTPGVPDLVDFQPPPELVLREAGEEVPDAGPREGVSFPWSRPPGQGEFRALNARLP | |
| | LNTDAYLSLQELQGQDPTHLV | |
| IL-15Rβ | AVNGTSQFTCFYNSRANISCVWSQDGALQDTSCQVHAWPDRRRWNQTCELLPVSQA | |
| ECD | SWACNLILGAPDSQKLTTVDIVTLRVLCREGVRWRVMAIQDFKPFENLRLMAPISL | |
| (27-240) | QVVHVETHRCNISWEISQASHYFERHLEFEARTLSPGHTWEEAPLLTLKQKQEWIC | |
| | LETLTPDTQYEFQVRVKPLQGEFTTWSPWSQPLAFRTKPAALGKDT | |

Thus, in certain embodiments, the cytokine receptor is an IL-15Rβ protein that comprises, consists, or consists essentially of an amino acid sequence selected from Table S10, or an active variant or fragment thereof that is at least 80, 85, 90, 95, 98, or 10000 identical to an amino acid sequence selected from Table S10, and which binds to an IL-15 portion of the activatable proprotein homodimer.

In certain embodiments, the IL-15β protein is a variant that comprises one or more amino acid alterations relative to wild-type IL-15Rβ, and which has reduced binding affinity to IL-15 relative to that of wild-type IL-15Rβ. For instance, in some embodiments, the IL-15Rβ protein variant has reduced binding affinity to IL-15 of about or at least about 2-fold, 5-fold, 10-fold, 50-fold, 100-fold, 1000-fold or more, relative to the binding affinity of IL-15Rβ to IL-15.

Any of the foregoing IL-15Rβ proteins can be combined with any of the binding moieties, corresponding IL-15 proteins, and linkers described herein, to generate one or more activatable proprotein or procytokine homodimers, or larger, multi-chain structures comprising the same.

Interleukin-21 Receptor (IL-21R). In certain embodiments, if the cytokine is an IL-21 protein, the cytokine receptor is an IL-21R protein, or a variant or fragment (e.g., extracellular domain or ECD) thereof. Exemplary human IL-21R amino acid sequences are provided in Table Sit below.

TABLE S11
Exemplary IL-21R Sequences

| | | SEQ ID |
|---|---|---|
| Name | Sequence | NO: |
| IL-21R | MPRGWAAPLLLLLLQGGWGCPDLVCYTDYLQTVICILEMWNLHPSTLTLTWQDQYE | |
| FL | ELKDEATSCSLHRSAHNATHATYTCHMDVFHFMADDIFSVNITDQSGNYSQECGSF | |
| | LLAESIKPAPPFNVTVTFSGQYNISWRSDYEDPAFYMLKGKLQYELQYRNRGDPWA | |
| | VSPRRKLISVDSRSVSLLPLEFRKDSSYELQVRAGPMPGSSYQGTWSEWSDPVIFQ | |
| | TQSEELKEGWNPHLLLLLLLVIVFIPAFWSLKTHPLWRLWKKIWAVPSPERFFMPL | |
| | YKGCSGDFKKWVGAPFTGSSLELGPWSPEVPSTLEVYSCHPPRSPAKRLQLTELQE | |
| | PAELVESDGVPKPSFWPTAQNSGGSAYSEERDRPYGLVSIDTVTVLDAEGPCTWPC | |
| | SCEDDGYPALDLDAGLEPSPGLEDPLLDAGTTVLSCGCVSAGSPGLGGPLGSLLDR | |
| | LKPPLADGEDWAGGLPWGGRSPGGVSESEAGSPLAGLDMDTFDSGFVGSDCSSPVE | |
| | CDFTSPGDEGPPRSYLRQWVVIPPPLSSPGPQAS | |
| IL-21R | CPDLVCYTDYLQTVICILEMWNLHPSTLTLTWQDQYEELKDEATSCSLHRSAHNAT | |
| mature | HATYTCHMDVFHFMADDIFSVNITDQSGNYSQECGSFLLAESIKPAPPFNVTVTFS | |
| w/o | GQYNISWRSDYEDPAFYMLKGKLQYELQYRNRGDPWAVSPRRKLISVDSRSVSLLP | |
| signal | LEFRKDSSYELQVRAGPMPGSSYQGTWSEWSDPVIFQTQSEELKEGWNPHLLLLLL | |
| peptide | LVIVFIPAFWSLKTHPLWRLWKKIWAVPSPERFFMPLYKGCSGDFKKWVGAPFTGS | |
| (20-538) | SLELGPWSPEVPSTLEVYSCHPPRSPAKRLQLTELQEPAELVESDGVPKPSFWPTA | |
| | QNSGGSAYSEERDRPYGLVSIDTVTVLDAEGPCTWPCSCEDDGYPALDLDAGLEPS | |
| | PGLEDPLLDAGTTVLSCGCVSAGSPGLGGPLGSLLDRLKPPLADGEDWAGGLPWGG | |
| | RSPGGVSESEAGSPLAGLDMDTFDSGFVGSDCSSPVECDFTSPGDEGPPRSYLRQW | |
| | WIPPPLSSPGPQAS | |
| IL-21R | CPDLVCYTDYLQTVICILEMWNLHPSTLTLTWQDQYEELKDEATSCSLHRSAHNAT | |
| ECD | HATYTCHMDVFHFMADDIFSVNITDQSGNYSQECGSFLLAESIKPAPPFNVTVTFS | |
| (20-232) | GQYNISWRSDYEDPAFYMLKGKLQYELQYRNRGDPWAVSPRRKLISVDSRSVSLLP | |
| | LEFRKDSSYELQVRAGPMPGSSYQGTWSEWSDPVIFQTQSEELKE | |
| IL-21R | CPDLVCYTDYLQTVICILEMWNLHPSTLTLTWQDQAEELKDEATSCSLHRSAHNAT | |
| ECD | HATYTCHMDVFHFMADDIFSVNITDQSGNYSQECGSFLLAESIKPAPPFNVTVTFS | |
| variant | GQYNISWRSDYEDPAFYMLKGKLQYELQYRNRGDPWAVSPRRKLISVDSRSVSLLP | |
| (20-232) | LEFRKDSSYELQVRAGPMPGSSYQGTWSEWSDPVIFQTQSEELKE | |
| IL-21R | CPDLVCYTDYLQTVICILEMWNLHPSTLTLTWQDQYEELKDEATSCSLHRSAHNAT | |
| ECD | HATYTCHMDVFHFAADDIFSVNITDQSGNYSQECGSFLLAESIKPAPPFNVTVTFS | |
| variant | GQYNISWRSDYEDPAFYMLKGKLQYELQYRNRGDPWAVSPRRKLISVDSRSVSLLP | |
| (20-232) | LEFRKDSSYELQVRAGPMPGSSYQGTWSEWSDPVIFQTQSEELKE | |
| IL-21R | CPDLVCYTDYLQTVICILEMWNLHPSTLTLTWQDQYEELKDEATSCSLHRSAHNAT | |
| ECD | HATYTCHMDVFHFMAADIFSVNITDQSGNYSQECGSFLLAESIKPAPPFNVTVTFS | |
| variant | GQYNISWRSDYEDPAFYMLKGKLQYELQYRNRGDPWAVSPRRKLISVDSRSVSLLP | |
| (20-232) | LEFRKDSSYELQVRAGPMPGSSYQGTWSEWSDPVIFQTQSEELKE | |
| IL-21R | CPDLVCYTDYLQTVICILEMWNLHPSTLTLTWQDQYEELKDEATSCSLHRSAHNAT | |
| ECD | HATYTCHMDVFHFMADAIFSVNITDQSGNYSQECGSFLLAESIKPAPPFNVTVTFS | |
| variant | GQYNISWRSDYEDPAFYMLKGKLQYELQYRNRGDPWAVSPRRKLISVDSRSVSLLP | |
| (20-232) | LEFRKDSSYELQVRAGPMPGSSYQGTWSEWSDPVIFQTQSEELKE | |
| IL-21R | CPDLVCYTDYLQTVICILEMWNLHPSTLTLTWQDQYEELKDEATSCSLHRSAHNAT | |
| ECD | HATYTCHMDVFHFMADDIFSVNITDQSGNYSQECGSFLLAESIKPAPPFNVTVTFS | |
| variant | GQYNISWRSDYEDPAFAMLKGKLQYELQYRNRGDPWAVSPRRKLISVDSRSVSLLP | |
| (20-232) | LEFRKDSSYELQVRAGPMPGSSYQGTWSEWSDPVIFQTQSEELKE | |
| IL-21R | CPDLVCYTDYLQTVICILEMWNLHPSTLTLTWQDQAEELKDEATSCSLHRSAHNAT | |
| ECD | HATYTCHMDVFHFMADDIFSVNITDQSGNYSQECGSFLLAESIKPAPPFNVTVTFS | |
| variant | GQYNISWRSDYEDPAFAMLKGKLQYELQYRNRGDPWAVSPRRKLISVDSRSVSLLP | |
| (20-232) | LEFRKDSSYELQVRAGPMPGSSYQGTWSEWSDPVIFQTQSEELKE | |

Thus, in certain embodiments, the cytokine receptor is an IL-21R protein that comprises, consists, or consists essentially of an amino acid sequence selected from Table S11, or an active variant or fragment thereof that is at least 80, 85, 90, 95, 98, or 100% identical to an amino acid sequence selected from Table S11, and which binds to an IL-21 portion of the activatable proprotein homodimer.

In certain embodiments, the IL-21R protein is a variant that comprises one or more amino acid alterations relative to wild-type IL-21R, and which has reduced binding affinity to IL-21 relative to that of wild-type IL-21R. For instance, in some embodiments, the IL-21R protein variant has reduced binding affinity to IL-21 of about or at least about 2-fold, 5-fold, 10-fold, 50-fold, 100-fold, 1000-fold or more, relative to the binding affinity of IL-21R to IL-21. In some embodiments, an IL-21R protein variant comprises or retains a substitution at any one or more of Y36, M70, D72, D73, and/or Y129, as defined by the mature IL-21R sequence, including any one or more of Y36A, M70A, D72A, D73A, and/or Y129A.

Any of the foregoing IL-21R proteins can be combined with any of the binding moieties, corresponding IL-21 proteins, and linkers described herein, to generate one or more activatable proprotein or procytokine homodimers, or larger, multi-chain structures comprising the same.

Interferon Receptors (IFNRs). In certain embodiments, if the cytokine is an IFN protein, the cytokine receptor is an IFNR protein, or a variant or fragment (e.g., extracellular domain or ECD) thereof.

In some embodiments, if the cytokine is a type I IFN, the cytokine receptor is IFNAR2, that is, the IFNAR2 chain of the interferon α/β receptor. Exemplary human IFNAR amino acid sequences are provided in Table S12 below.

TABLE S12
IFNAR Sequences

| | | SEQ ID |
|---|---|---|
| Name | Sequence | NO: |
| IFNAR FL | MLLSQNAFIFRSLNLVLMVYISLVFGISYDSPDYTDESCTFKISLRNFRSILSWEL | |
| | KNHSIVPTHYTLLYTIMSKPEDLKVVKNCANTTRSFCDLTDEWRSTHEAYVTVLEG | |
| | FSGNTTLFSCSHNFWLAIDMSFEPPEFEIVGFTNHINVMVKFPSIVEEELQFDLSL | |
| | VIEEQSEGIVKKHKPEIKGNMSGNFTYIIDKLIPNTNYCVSVYLEHSDEQAVIKSP | |
| | LKCTLLPPGQESESAESAKIGGIITVFLIALVLTSTIVTLKWIGYICLRNSLPKVL | |
| | NFHNFLAWPFPNLPPLEAMDMVEVIYINRKKKVWDYNYDDESDSDTEAAPRTSGGG | |
| | YTMHGLTVRPLGQASATSTESQLIDPESEEEPDLPEVDVELPTMPKDSPQQLELLS | |
| | GPCERRKSPLQDPFPEEDYSSTEGSGGRITFNVDLNSVFLRVLDDEDSDDLEAPLM | |
| | LSSHLEEMVDPEDPDNVQSNHLLASGEGTQPTFPSPSSEGLWSEDAPSDQSDTSES | |
| | DVDLGDGYIMR | |
| IFNAR | ISYDSPDYTDESCTFKISLRNFRSILSWELKNHSIVPTHYTLLYTIMSKPEDLKVV | |
| mature | KNCANTTRSFCDLTDEWRSTHEAYVTVLEGFSGNTTLFSCSHNFWLAIDMSFEPPE | |
| (27-515) | FEIVGFTNHINVMVKFPSIVEEELQFDLSLVIEEQSEGIVKKHKPEIKGNMSGNFT | |
| | YIIDKLIPNTNYCVSVYLEHSDEQAVIKSPLKCTLLPPGQESESAESAKIGGIITV | |
| | FLIALVLTSTIVTLKWIGYICLRNSLPKVLNFHNFLAWPFPNLPPLEAMDMVEVIY | |
| | INRKKKVWDYNYDDESDSDTEAAPRTSGGGYTMHGLTVRPLGQASATSTESQLIDP | |
| | ESEEEPDLPEVDVELPTMPKDSPQQLELLSGPCERRKSPLQDPFPEEDYSSTEGSG | |
| | GRITFNVDLNSVFLRVLDDEDSDDLEAPLMLSSHLEEMVDPEDPDNVQSNHLLASG | |
| | EGTQPTFPSPSSEGLWSEDAPSDQSDTSESDVDLGDGYIMR | |
| IFNAR | ISYDSPDYTDESCTFKISLRNFRSILSWELKNHSIVPTHYTLLYTIMSKPEDLKVV | |
| ECD | KNCANTTRSFCDLTDEWRSTHEAYVTVLEGFSGNTTLFSCSHNFWLAIDMSFEPPE | |
| (27-243) | FEIVGFTNHINVMVKFPSIVEEELQFDLSLVIEEQSEGIVKKHKPEIKGNMSGNFT | |
| | YIIDKLIPNTNYCVSVYLEHSDEQAVIKSPLKCTLLPPGQESESAESAK | |
| IFNAR | ESCTFKISLRNFRSILSWELKNHSIVPTHYTLLYTIMSKPEDLKVVKNCANTTRSF | |
| ECD | CDLTDEWRSTHEAYVTVLEGFSGNTTLFSCSHNFWLAIDMSFEPPEFEIVGFTNHI | |
| fragment | NVMVKFPSIVEEELQFDLSLVIEEQSEGIVKKHKPEIKGNMSGNFTYIIDKLIPNT | |
| (37-232) | NYCVSVYLEHSDEQAVIKSPLKCTLLPP | |
| IFNAR | ESCTFKISLRNFRSILSWELKNHSIVPTHYTLLYTIASKPEDLKVVKNCANTTRSF | |
| ECD | CDLTDEWRSTHEAYVTVLEGFSGNTTLFSCSHNFWLAIDMSFEPPEFEIVGFTNHI | |
| variant | NVMVKFPSIVEEELQFDLSLVIEEQSEGIVKKHKPEIKGNMSGNFTYIIDKLIPNT | |
| (37-232) | NYCVSVYLEHSDEQAVIKSPLKCTLLPP | |
| IFNAR | ESCTFKISLRNFRSILSWELKNHSIVPTHYTLLYTIMSKPEDLKVVKNCANTTRSF | |
| ECD | CDLTDEWRSTHAAYVTVLEGFSGNTTLFSCSHNFWLAIDMSFEPPEFEIVGFTNHI | |
| variant | NVMVKFPSIVEEELQFDLSLVIEEQSEGIVKKHKPEIKGNMSGNFTYIIDKLIPNT | |
| (37-232) | NYCVSVYLEHSDEQAVIKSPLKCTLLPP | |
| IFNAR | ESCTFKISLRNFRSILSWELKNHSIVPTHYTLLYTIVSKPEDLKVVKNCANTTRSF | |
| ECD | CDLTDEWRSTHAAYVTVLEGFSGNTTLFSCSHNFWLAIDMSFEPPEFEIVGFTNHI | |
| variant | NVMVKFPSIVEEELQFDLSLVIEEQSEGIVKKHKPEIKGNMSGNFTYIIDKLIPNT | |
| (37-232) | NYCVSVYLEHSDEQAVIKSPLKCTLLPP | |

Thus, in certain embodiments, a cytokine receptor is an IFNAR protein that comprises, consists, or consists essentially of an amino acid sequence selected from Table S12, or an active variant or fragment thereof that is at least 80, 85, 90, 95, 98, or 10000 identical to an amino acid sequence selected from Table S12, and binds to a type I IFN portion of the activatable proprotein homodimer.

In certain embodiments, the IFNAR is a variant that comprises one or more amino acid alterations relative to wild-type IFNAR, and which has reduced binding affinity to the type I IFN relative to that of the wild-type IFNAR. For instance, in some embodiments, the IFNAR has reduced binding affinity to the type I IFN of about or at least about 2-fold, 5-fold, 10-fold, 50-fold, 100-fold, 1000-fold or more, relative to the binding affinity of wild-type IFNAR to the type I IFN. In some embodiments, an IFNAR variant comprises or retains a substitution at M47 and/or E78, as defined by the mature IFNAR sequence, including any one or more of an M47A, M47V, and/or E78A substitution.

In some embodiments, if the cytokine is a type II IFN, the cytokine receptor is IFNGR1. Exemplary human IFNGR1 amino acid sequences are provided in Table S13 below.

TABLE S13
Exemplary IFNGR1 Sequences

| | | SEQ ID |
|---|---|---|
| Name | Sequence | NO: |
| IFNGR1 | MALLFLLPLVMQGVSRAEMGTADLGPSSVPTPTNVTIESYNMNPIVYWEYQIMPQV | |
| FL | PVFTVEVKNYGVKNSEWIDACINISHHYCNISDHVGDPSNSLWVRVKARVGQKESA | |
| | YAKSEEFAVCRDGKIGPPKLDIRKEEKQIMIDIFHPSVFVNGDEQEVDYDPETTCY | |
| | IRVYNVYVRMNGSEIQYKILTQKEDDCDEIQCQLAIPVSSLNSQYCVSAEGVLHVW | |
| | GVTTEKSKEVCITIFNSSIKGSLWIPVVAALLLFLVLSLVFICFYIKKINPLKEKS | |
| | IILPKSLISVVRSATLETKPESKYVSLITSYQPFSLEKEVVCEEPLSPATVPGMHT | |
| | EDNPGKVEHTEELSSITEVVTTEENIPDVVPGSHLTPIERESSSPLSSNQSEPGSI | |
| | ALNSYHSRNCSESDHSRNGFDTDSSCLESHSSLSDSEFPPNNKGEIKTEGQELITV | |
| | IKAPTSFGYDKPHVLVDLLVDDSGKESLIGYRPTEDSKEFS | |
| IFNGR1 | EMGTADLGPSSVPTPTNVTIESYNMNPIVYWEYQIMPQVPVFTVEVKNYGVKNSEW | |
| Mature | IDACINISHHYCNISDHVGDPSNSLWVRVKARVGQKESAYAKSEEFAVCRDGKIGP | |
| | PKLDIRKEEKQIMIDIFHPSVFVNGDEQEVDYDPETTCYIRVYNVYVRMNGSEIQY | |
| | KILTQKEDDCDEIQCQLAIPVSSLNSQYCVSAEGVLHVWGVTTEKSKEVCITIFNS | |
| | SIKGSLWIPVVAALLLFLVLSLVFICFYIKKINPLKEKSIILPKSLISVVRSATLE | |
| | TKPESKYVSLITSYQPFSLEKEVVCEEPLSPATVPGMHTEDNPGKVEHTEELSSIT | |
| | EVVTTEENIPDVVPGSHLTPIERESSSPLSSNQSEPGSIALNSYHSRNCSESDHSR | |
| | NGFDTDSSCLESHSSLSDSEFPPNNKGEIKTEGQELITVIKAPTSFGYDKPHVLVD | |
| | LLVDDSGKESLIGYRPTEDSKEFS | |
| IFNGR1 | EMGTADLGPSSVPTPTNVTIESYNMNPIVYWEYQIMPQVPVFTVEVKNYGVKNSEW | |
| ECD | IDACINISHHYCNISDHVGDPSNSLWVRVKARVGQKESAYAKSEEFAVCRDGKIGP | |
| (18-245) | PKLDIRKEEKQIMIDIFHPSVFVNGDEQEVDYDPETTCYIRVYNVYVRMNGSEIQY | |
| | KILTQKEDDCDEIQCQLAIPVSSLNSQYCVSAEGVLHVWGVTTEKSKEVCITIFNS | |
| | SIKG | |

Thus, in certain embodiments, a cytokine receptor is an IFNGR1 protein that comprises, consists, or consists essentially of an amino acid sequence selected from Table S13, or an active variant or fragment thereof that is at least 80, 85, 90, 95, 98, or 100 identical to an amino acid sequence selected from Table S13, and binds to a type II IFN portion of the activatable proprotein homodimer.

In certain embodiments, the IFNGR1 is a variant that comprises one or more amino acid alterations relative to wild-type IFNGR1, and which has reduced binding affinity to the type II IFN relative to that of wild-type IFNGR1. For instance, in some embodiments, the IFNGR1 variant has reduced binding affinity to the type II IFN of about or at least about 2-fold, 5-fold, 10-fold, 50-fold, 100-fold, 1000-fold or more, relative to the binding affinity of wild-type IFNGR1 to the type II IFN.

In some embodiments, if the cytokine is a type III IFN, the cytokine receptor is IL10Rβ or IFN-λR1 (IFNLR1). Exemplary human IL10Rβ and IFN-λR1 amino acid sequences are provided in Table S13 below.

TABLE S14

| | | SEQ ID |
|---|---|---|
| Name | Sequence | NO: |
| IFNLR1 | MAGPERWGPLLLCLLQAAPGRPRLAPPQNVTLLSQNFSVYLTWLPGLGNPQDVTYF | |
| FL | VAYQSSPTRRRWREVEECAGTKELLCSMMCLKKQDLYNKFKGRVRTVSPSSKSPWV | |
| | ESEYLDYLFEVEPAPPVLVLTQTEEILSANATYQLPPCMPPLDLKYEVAFWKEGAG | |
| | NKTLFPVTPHGQPVQITLQPAASEHHCLSARTIYTFSVPKYSKFSKPTCFLLEVPE | |
| | ANWAFLVLPSLLILLLVIAAGGVIWKTLMGNPWFQRAKMPRALDFSGHTHPVATFQ | |
| | PSRPESVNDLFLCPQKELTRGVRPTPRVRAPATQQTRWKKDLAEDEEEEDEEDTED | |
| | GVSFQPYIEPPSFLGQEHQAPGHSEAGGVDSGRPRAPLVPSEGSSAWDSSDRSWAS | |
| | TVDSSWDRAGSSGYLAEKGPGQGPGGDGHQESLPPPEFSKDSGFLEELPEDNLSSW | |
| | ATWGTLPPEPNLVPGGPPVSLQTLTFCWESSPEEEEEARESEIEDSDAGSWGAEST | |
| | QRTEDRGRTLGHYMAR | |
| IFNLR1 | RPRLAPPQNVTLLSQNFSVYLTWLPGLGNPQDVTYFVAYQSSPTRRRWREVEECAG | |
| Mature | TKELLCSMMCLKKQDLYNKFKGRVRTVSPSSKSPWVESEYLDYLFEVEPAPPVLVL | |
| | TQTEEILSANATYQLPPCMPPLDLKYEVAFWKEGAGNKTLFPVTPHGQPVQITLQP | |
| | AASEHHCLSARTIYTFSVPKYSKFSKPTCFLLEVPEANWAFLVLPSLLILLLVIAA | |
| | GGVIWKTLMGNPWFQRAKMPRALDFSGHTHPVATFQPSRPESVNDLFLCPQKELTR | |
| | GVRPTPRVRAPATQQTRWKKDLAEDEEEEDEEDTEDGVSFQPYIEPPSFLGQEHQA | |
| | PGHSEAGGVDSGRPRAPLVPSEGSSAWDSSDRSWASTVDSSWDRAGSSGYLAEKGP | |
| | GQGPGGDGHQESLPPPEFSKDSGFLEELPEDNLSSWATWGTLPPEPNLVPGG | |
| IFNLR1 | RPRLAPPQNVTLLSQNFSVYLTWLPGLGNPQDVTYFVAYQSSPTRRRWREVEECAG | |
| ECD | TKELLCSMMCLKKQDLYNKFKGRVRTVSPSSKSPWVESEYLDYLFEVEPAPPVLVL | |
| (21-228) | TQTEEILSANATYQLPPCMPPLDLKYEVAFWKEGAGNKTLFPVTPHGQPVQITLQP | |
| | AASEHHCLSARTIYTFSVPKYSKFSKPTCFLLEVPEANWA | |
| IL10Rβ | MAWSLGSWLGGCLLVSALGMVPPPENVRMNSVNFKNILQWESPAFAKGNLTFTAQY | |
| FL | LSYRIFQDKCMNTTLTECDFSSLSKYGDHTLRVRAEFADEHSDWVNITFCPVDDTI | |
| | IGPPGMQVEVLADSLHMRFLAPKIENEYETWTMKNVYNSWTYNVQYWKNGTDEKFQ | |
| | ITPQYDFEVLRNLEPWTTYCVQVRGFLPDRNKAGEWSEPVCEQTTHDETVPSWMVA | |
| | VILMASVFMVCLALLGCFALLWCVYKKTKYAFSPRNSLPQHLKEFLGHPHHNTLLF | |
| | FSFPLSDENDVFDKLSVIAEDSESGKQNPGDSCSLGTPPGQGPQS | |
| IL10Rβ | MVPPPENVRMNSVNFKNILQWESPAFAKGNLTFTAQYLSYRIFQDKCMNTTLTECD | |
| mature | FSSLSKYGDHTLRVRAEFADEHSDWVNITFCPVDDTIIGPPGMQVEVLADSLHMRF | |
| | LAPKIENEYETWTMKNVYNSWTYNVQYWKNGTDEKFQITPQYDFEVLRNLEPWTTY | |
| | CVQVRGFLPDRNKAGEWSEPVCEQTTHDETVPSWMVAVILMASVFMVCLALLGCFA | |
| | LLWCVYKKTKYAFSPRNSLPQHLKEFLGHPHHNTLLFFSFPLSDENDVFDKLSVIA | |
| | EDSESGKQNPGDSCSLGTPPGQGPQS | |
| IL10Rβ | MVPPPENVRMNSVNFKNILQWESPAFAKGNLTFTAQYLSYRIFQDKCMNTTLTECD | |
| ECD (20- | FSSLSKYGDHTLRVRAEFADEHSDWVNITFCPVDDTIIGPPGMQVEVLADSLHMRF | |
| 220) | LAPKIENEYETWTMKNVYNSWTYNVQYWKNGTDEKFQITPQYDFEVLRNLEPWTTY | |
| | CVQVRGFLPDRNKAGEWSEPVCEQTTHDETVPS | |

Thus, in certain embodiments, a cytokine receptor is an IL10Rβ or IFN-λR1 protein that comprises, consists, or consists essentially of an amino acid sequence selected from Table S14, or an active variant or fragment thereof that is at least 80, 85, 90, 95, 98, or 10000 identical to an amino acid sequence selected from Table S14, and binds to a type III IFN portion of the activatable proprotein homodimer.

In certain embodiments, the IL10Rβ or IFN-λR1 is a variant that comprises one or more amino acid alterations relative to wild-type IL10Rβ or IFN-λR1, and which has reduced binding affinity to the type III IFN relative to that of the wild-type IL10Rβ or IFN-λR1 or a complex thereof. For instance, in some embodiments, the IL10Rβ or IFN-λR1 variant has reduced binding affinity to the type III IFN of about or at least about 2-fold, 5-fold, 10-fold, 50-fold, 100-fold, 1000-fold or more, relative to the binding affinity of wild-type IL10Rβ or IFN-λR1 or a complex thereof to the type III IFN.

Any of the foregoing IFNR proteins can be combined with any of the binding moieties, corresponding IFNs, and linkers described herein, to generate one or more activatable proprotein or procytokine homodimers, or larger, multi-chain structures comprising the same.

Binding Moieties. As noted above, the activatable proprotein homodimers described herein comprise a first polypeptide and a second polypeptide, each of which comprises a “binding moiety”. In some aspects, the binding moiety of the first polypeptide binds to the binding moiety of the second polypeptide, which further stabilizes the binding interaction between the first and second polypeptides.

The binding moieties of the first and second polypeptides can be the same or substantially same, interacting together as a “binding moiety homodimer”, or they can be different, interacting together as a “binding moiety heterodimer”. In some embodiments, the binding moieties do not bind to any of the cytokine or cytokine receptor portions of the homodimers described herein.

General examples of binding moieties are provided in Table M1 below.

TABLE M1
Exemplary Binding Moieties

Short peptide
Leucine zipper peptide
VH
VL
VH-CH1
VL-CL
VH-CL
VL-CH1
CH3
CH2CH3
Fab-CH3
Fab-CH2CH3
Antigen binding domain-CH3
Antigen binding domain-CH2CH3
CH3 variant
CH2CH3 variant
Fab-CH3 variant
Fab-CH2CH3 variant
Antigen binding domain-CH3 variant
Antigen binding domain-CH2CH3 variant

Thus, in certain embodiments, a binding moiety is selected from Table M1.

In particular embodiments, a binding moiety comprises an antigen binding domain of an immunoglobulin, including antigen binding fragments and variants thereof, such as a VL domain and/or a VH domain. In some embodiments, an antigen binding domain does not bind to an antigen, for example, a human antigen. In some embodiments, an antigen binding domain binds to an antigen, for example, a human antigen.

In some embodiments, a binding moiety comprises a constant domain of an immunoglobulin, or a fragment or variant thereof. For example, in certain embodiments a binding moiety comprises a CH1, CH2, CH3, CH1CH3, CH2CH3, CH1CH2CH3, and/or CL domain of an immunoglobulin, including fragments and variants thereof, and combinations thereof. In some instances, the light chain (CL) is a lambda or kappa chain. In some embodiments, the constant domains present in a binding moiety of an activatable proprotein homodimer provided herein is glycosylated. In some embodiments, the glycosylation is N-glycosylation. In some embodiments, the glycosylation is O-glycosylation.

In specific embodiments, a binding moiety comprises, in an N- to C-terminal orientation: (1) an antigen binding domain of an immunoglobulin, including antigen binding fragments and variants thereof, and (2) an immunoglobulin constant domain, including fragments and variants thereof, for example, a CH1, CH2, CH3, CH1CH3, CH2CH3, CH1CH2CH3, and/or CL domain of an immunoglobulin, including combinations thereof. In specific embodiments, a binding moiety comprises, consists, or consists essentially of a CH2CH3 domain of an immunoglobulin.

The immunoglobulin domains used herein (antigen-binding domains, constant domains) optionally comprise IgG domains. However, certain embodiments comprise alternate immunoglobulins such as IgM, IgA, IgD, and IgE. Furthermore, all possible isotypes of the various immunoglobulins are also encompassed within the current embodiments. Thus, IgG1, IgG2, IgG3, etc., are all possible molecules in the binding domains. In addition to choice in selection of the type of immunoglobulin and isotype, certain embodiments comprise various hinge regions (or functional equivalents thereof). Such hinge regions provide flexibility between the different domains of the proproteins described herein. In some embodiments, the immunoglobulin portion of the binding domain (or larger masking moiety) is from an immunoglobulin class selected from IgG1, IgG2, IgG3, IgG4, IgD, IgA, and IgM.

Linkers. As noted above, in certain embodiments, an activatable proprotein (or procytokine) homodimer comprises a first linker and a second linker, for example, peptide linkers, including flexible linkers. In some embodiments, at least one of the linkers is a cleavable linker, for example, a cleavable linker that comprises a protease cleavage site. In some embodiments, at least one of the linkers is a non-cleavable linker, that is, a physiologically-stable linker, or a stable linker.

In some embodiments, the first linker and/or second linker are about 1-50 1-40, 1-30, 1-20, 1-10, 1-5, 1-4, 1-3 amino acids in length, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acids in length. In some embodiments, the first linker is a cleavable linker, for example, a cleavable linker that comprises a protease cleavage site, and the second linker is a non-cleavable linker, such as a physiologically-stable, flexible linker. In some embodiments, the first linker is a non-cleavable linker, such as a physiologically-stable, flexible linker, and the second linker is a cleavable linker, for example, a cleavable linker that comprises a protease cleavage site. In some embodiments, the cleavable linker is a flexible linker.

In some embodiments, a cleavable linker comprises at least one protease cleavage site, or is a low pH-sensitive linker. Suitable protease cleavages sites and self-cleaving peptides are known to the skilled person (see, e.g., Ryan et al., J. Gener. Virol. 78:699-722, 1997; and Scymczak et al., Nature Biotech. 5:589-594, 2004). In some embodiments, the protease cleavage site is cleavable by a protease selected from one or more of a metalloprotease, a serine protease, a cysteine protease, and an aspartic acid protease. In particular embodiments, the protease cleavage site is cleavable by a protease selected from one or more of MMP1, MMP2, MMP3, MMP4, MMP5, MMP6, MMP7, MMP8, MMP9, MMP10, MMP11, MMP12, MMP13, MMP14, TEV protease, matriptase, uPA, FAP, Legumain, PSA, Kallikrein, Cathepsin A, and Cathepsin B.

Exemplary stable linker sequences, including flexible linkers, are provided in Table L1.

TABLE L1
Exemplary stable linkers

| | | SEQ ID |
|---|---|---|
| Name | Sequence | NO: |
| | [G]ₓ | |
| | [S]ₓ | |
| | [N]ₓ | |
| | [GS]ₓ | |
| | [GGS]ₓ | |
| | [GSS]ₓ | |
| | [GSGS]ₓ | |
| | [GGSG]ₓ | |
| | [GGGS]ₓ | |
| | [GSGGG]ₓ | |
| | [GGGGS]ₓ | |
| | [GN]ₓ | |
| | [GGN]ₓ | |
| | [GNN]ₓ | |
| | [GNGN]ₓ | |
| | [GGNG]ₓ | |
| | [GGGN]ₓ | |
| | [GGGGN]ₓ | |
| | DGGGS | |
| | TGEKP | |
| | GGRR | |
| | EGKSSGSGSESKVD | |
| | KESGSVSSEQLAQFRSLD | |
| | GGRRGGGS | |
| | LRQRDGERP | |
| | LRQKDGGGSERP | |
| | LRQKD(GGGS)₂ERP | |

where ₓ is 1, 2, 3, 4, 5, 6, 7, 8, 9, 10, 11, 12, 13, 14, 15, 16, 17, 18, 19, or 20

Thus, in certain embodiment, a stable and/or flexible linker is selected from Table L1. In particular embodiments, flexible linkers can be rationally designed using a computer program capable of modeling both DNA-binding sites and the peptides themselves (Desjarlais & Berg, PNAS. 90:2256-2260, 1993; and PNAS. 91:11099-11103, 1994) or by phage display methods.

Exemplary cleavable linker sequences are provided in Table L2.

TABLE L2
Exemplary cleavable linkers

| | | SEQ ID |
|---|---|---|
| Name | Sequence | NO: |
| | GGGSENLYFQG | |
| | PLGLAG | |
| | SGRSDNR | |
| | PLGLAGSGRSDNR | |
| | GSLSGRSDNHGS | |
| | LGGSGRSANA | |
| | LSGRSANAG | |
| | GPLGLAGRSANA | |
| | PLGLSGRSANAGPA | |
| | PLGLAGRSANAGPA | |
| | GPLGLSGRSANAGPASG | |
| | GPLGLAGRSANAGPASG | |
| | SGPLGLAGRSANAGPAS | |
| | SGPASGRSANAPLGLAG | |
| | GPASGRSANAPLGLAGS | |
| | GPLGLAGRSANPGPASG | |
| | GPLGLAGRSDNHGPASG | |
| | GPLGLAGRSDNPGPASG | |
| | GPLGLAGRSENPGPASG | |
| | GPLGLAGRSDNLGPASG | |
| | GPLGLAGRNAQVGPASG | |
| | LSGRSDNA | |
| | LSGRSDND | |
| | LSGRSDNE | |
| | LSGRSDNF | |
| | LSGRSDNG | |
| | LSGRSDNI | |
| | LSGRSDNK | |
| | LSGRSDNL | |
| | LSGRSDNM | |
| | LSGRSDNN | |
| | LSGRSDNP | |
| | LSGRSDNQ | |
| | LSGRSDNR | |
| | LSGRSDNS | |
| | LSGRSDNT | |
| | LSGRSDNV | |
| | LSGRSDNW | |
| | LSGRSDNY | |
| | LSGRSAND | |
| | LSGRSANE | |
| | LSGRSANF | |
| | LSGRSANG | |
| | LSGRSANH | |
| | LSGRSANI | |
| | LSGRSANK | |
| | LSGRSANL | |
| | LSGRSANM | |
| | LSGRSANN | |
| | LSGRSANP | |
| | LSGRSANQ | |
| | LSGRSANR | |
| | LSGRSANS | |
| | LSGRSANT | |
| | LSGRSANV | |
| | LSGRSANW | |
| | LSGRSANY | |
| | PLGLAGRSDNHS | |
| | PLGLAGSGRSDNRGA | |
| | PLGLAGSGRSDNQGA | |
| | PLGLAGSGRSDNYGA | |
| | GPLGLAGSGRSDNQG | |
| | PLGLAGSGRSDNQ | |
| | PLGLAGSGRSDNH | |
| | PLGLAGSGRSDNT | |
| | SGRSDNH | |

Thus, in certain embodiment, a cleavable linker is selected from Table L2. In particular embodiments, a cleavable linker has a half life at pH 7.4, 25° C., for example, at physiological pH, human body temperature (e.g., in vivo, in serum, in a given tissue), of about or less than about 30 minutes, 1 hour, 2 hours, 3 hours, 4 hours, 5 hours, 6 hours, 12 hours, 18 hours, 24 hours, 36 hours, 48 hours, 72 hours, or 96 hours, or any intervening half-life.

Additional examples of cleavable linkers include an amino acid sequence cleaved by a serine protease such as thrombin, chymotrypsin, trypsin, elastase, kallikrein, or subtilisin. Illustrative examples of thrombin-cleavable amino acid sequences include, but are not limited to: -Gly-Arg-Gly-Asp- (SEQ ID NO: _), -Gly-Gly-Arg-, -Gly-Arg-Gly-Asp-Asn-Pro- (SEQ ID NO: _), -Gly-Arg-Gly-Asp-Ser- (SEQ ID NO: _), -Gly-Arg-Gly-Asp-Ser-Pro-Lys- (SEQ ID NO: _), -Gly-Pro-Arg-, -Val-Pro-Arg-, and -Phe-Val-Arg-. Illustrative examples of elastase-cleavable amino acid sequences include, but are not limited to: -Ala-Ala-Ala-, -Ala-Ala-Pro-Val- (SEQ ID NO: _), -Ala-Ala-Pro-Leu- (SEQ ID NO: _), -Ala-Ala-Pro-Phe- (SEQ ID NO: 121), -Ala-Ala-Pro-Ala- (SEQ ID NO: _), and -Ala-Tyr-Leu-Val- (SEQ ID NO: _).

Cleavable linkers also include amino acid sequences that can be cleaved by a matrix metalloproteinase such as collagenase, stromelysin, and gelatinase. Illustrative examples of matrix metalloproteinase-cleavable amino acid sequences include, but are not limited to: -Gly-Pro-Y-Gly-Pro-Z- (SEQ ID NO: _), -Gly-Pro-, Leu-Gly-Pro-Z- (SEQ ID NO: _), -Gly-Pro-Ile-Gly-Pro-Z- (SEQ ID NO: _), and -Ala-Pro-Gly-Leu-Z- (SEQ ID NO: _), where Y and Z are amino acids. Illustrative examples of collagenase-cleavable amino acid sequences include, but are not limited to: -Pro-Leu-Gly-Pro-D-Arg-Z- (SEQ ID NO: _), -Pro-Leu-Gly-Leu-Leu-Gly-Z- (SEQ ID NO: _), -Pro-Gln-Gly-Ile-Ala-Gly-Trp- (SEQ ID NO: _), -Pro-Leu-Gly-Cys(Me)-His- (SEQ ID NO: _), -Pro-Leu-Gly-Leu-Tyr-Ala- (SEQ ID NO: _), -Pro-Leu-Ala-Leu-Trp-Ala-Arg- (SEQ ID NO: _), and -Pro-Leu-Ala-Tyr-Trp-Ala-Arg- (SEQ ID NO: 134), where Z is an amino acid. An illustrative example of a stromelysin-cleavable amino acid sequence is -Pro-Tyr-Ala-Tyr-Tyr-Met-Arg- (SEQ ID NO: _); and an example of a gelatinase-cleavable amino acid sequence is -Pro-Leu-Gly-Met-Tyr-Ser-Arg- (SEQ ID NO: _).

Cleavable linkers also include amino acid sequences that can be cleaved by an angiotensin converting enzyme, such as, for example, -Asp-Lys-Pro-, -Gly-Asp-Lys-Pro- (SEQ ID NO: _), and -Gly-Ser-Asp-Lys-Pro- (SEQ ID NO: _). Cleavable linkers also include amino acid sequences that can be degraded by cathepsin B, such as, for example, Val-Cit, Ala-Leu-Ala-Leu- (SEQ ID NO: _), Gly-Phe-Leu-Gly- (SEQ ID NO: _) and Phe-Lys.

Any one or more of the foregoing linkers can be any of the binding moieties, cytokines, and cytokine receptors described herein, to generate one or more activatable proprotein or procytokine homodimers, or larger, multi-chain structures comprising the same.

Exemplary activatable proprotein homodimers are provided in Table S15.

TABLE S15
Exemplary Activatable Proprotein Homodimer Sequences

| | | |
|---|---|---|
| | | SEQ ID |
| Name | Sequence | NO: |

P2989 IL-7/IL-7R

| | |
|---|---|
| Chains 1 | ESGYAQNGDLEDAELDDYSFSCYSQLEVNGSQHSLTCAFEDPDVNTTNLEFE |
| and 2 | ICGALVEVKCLNFRKLQEIYFIETKKFLLIGKSNICVKVGEKSLTCKKIDLT |
| | TIVKPEAPFDLSVVYREGANDFVVTFNTSHLQKKYVKVLMHDVAYRQEKDEN |
| | KWTHVNLSSTKLTLLQRKLQPAAMYEIKVRSIPDHYFKGFWSEWSPSYYFRT |
| | PEINNSSGEMD [symbol] DCDIEGKDGKQYESVLMVSIDQLLDSMKEIGSN |
| | CLNNEFNFFKRHICDANKEGMFLFRAARKLRQFLKMNSTGDFDLHLLKVSEG |
| | TTILLNCTGQVKGRKPAALGAAQPTKSLEENKSLKEQKKLNDLCFLKRLLQE |
| | IKTCWNKILMGTKEH [symbol] EPKSSDKTHTCPPCPAPEAAGGPSVF |
| | LFPPKPKDTLMISRTPEVTCVVVDVSHEDPEVKFNWYVDGVEVHNAKTKPRE |
| | EQYNSTYRVVSVLTVLHQDWLNGKEYKCKVSNKALAAPIEKTISKAKGQPRE |
| | PQVYTLPPSRDELTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |

P2990 IL-7/IL-7R

| | |
|---|---|
| Chains 1 | ESGYAQNGDLEDAELDDYSFSCYSQLEVNGSQHSLTCAFEDPDVNTTNLEFE |
| and 2 | ICGALVEVKCLNFRKLQEIYFIETAKFLLIGKSNICVKVGEKSLTCKKIDLT |
| | TIVKPEAPFDLSVVYREGANDFVVTFNTSHLQKKYVKVLMHDVAYRQEKDEN |
| | KWTHVNLSSTKLTLLQRKLQPAAMYEIKVRSIPDHYFKGFWSEWSPSYYFRT |
| | PEINNSSGEMD [symbol] DCDIEGKDGKQYESVLMVSIDQLLDSMKEIGSN |
| | CLNNEFNFFKRHICDANKEGMFLFRAARKLRQFLKMNSTGDFDLHLLKVSEG |
| | TTILLNCTGQVKGRKPAALGAAQPTKSLEENKSLKEQKKLNDLCFLKRLLQE |
| | IKTCWNKILMGTKEH [symbol] EPKSSDKTHTCPPCPAPEAAGGPSVF |
| | LFPPKPKDTLMISRTPEVTCVVVDVSHEDPEVKFNWYVDGVEVHNAKTKPRE |
| | EQYNSTYRVVSVLTVLHQDWLNGKEYKCKVSNKALAAPIEKTISKAKGQPRE |
| | PQVYTLPPSRDELTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |

P2991 IL-7/IL-7R

| | |
|---|---|
| Chains 1 | ESGYAQNGDLEDAELDDYSFSCYSQLEVNGSQHSLTCAFEDPDVNTTNLEFE |
| and 2 | ICGALVEVKCLNFRKLQEIYFIETKKFALIGKSNICVKVGEKSLTCKKIDLT |
| | TIVKPEAPFDLSVVYREGANDFVVTFNTSHLQKKYVKVLMHDVAYRQEKDEN |
| | KWTHVNLSSTKLTLLQRKLQPAAMYEIKVRSIPDHYFKGFWSEWSPSYYFRT |
| | PEINNSSGEMD [symbol] DCDIEGKDGKQYESVLMVSIDQLLDSMKEIGSN |
| | CLNNEFNFFKRHICDANKEGMFLFRAARKLRQFLKMNSTGDFDLHLLKVSEG |
| | TTILLNCTGQVKGRKPAALGAAQPTKSLEENKSLKEQKKLNDLCFLKRLLQE |
| | IKTCWNKILMGTKEH [symbol] EPKSSDKTHTCPPCPAPEAAGGPSVF |
| | LFPPKPKDTLMISRTPEVTCVVVDVSHEDPEVKFNWYVDGVEVHNAKTKPRE |
| | EQYNSTYRVVSVLTVLHQDWLNGKEYKCKVSNKALAAPIEKTISKAKGQPRE |
| | PQVYTLPPSRDELTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |

P2992 IL-7/IL-7R

| | |
|---|---|
| Chains 1 | ESGYAQNGDLEDAELDDYSFSCYSQLEVNGSQHSLTCAFEDPDVNTTNLEFE |
| and 2 | ICGALVEVKCLNFRKLQEIYFIETKKFLLIGKSNICVKVGEKSLTCKKIDLT |
| | TIVKPEAPFDLSVVYREGANDFVVTFNTSHLQKAAYKVLMHDVAYRQEKDEN |
| | KWTHVNLSSTKLTLLQRKLQPAAMYEIKVRSIPDHYFKGFWSEWSPSYYFRT |
| | PEINNSSGEMD [symbol] DCDIEGKDGKQYESVLMVSIDQLLDSMKEIGSN |
| | CLNNEFNFFKRHICDANKEGMFLFRAARKLRQFLKMNSTGDFDLHLLKVSEG |
| | TTILLNCTGQVKGRKPAALGAAQPTKSLEENKSLKEQKKLNDLCFLKRLLQE |
| | IKTCWNKILMGTKEH [symbol] EPKSSDKTHTCPPCPAPEAAGGPSVF |
| | LFPPKPKDTLMISRTPEVTCVVVDVSHEDPEVKFNWYVDGVEVHNAKTKPRE |
| | EQYNSTYRVVSVLTVLHQDWLNGKEYKCKVSNKALAAPIEKTISKAKGQPRE |
| | PQVYTLPPSRDELTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |

P2993 IL-7/IL-7R

| | |
|---|---|
| Chains 1 | ESGYAQNGDLEDAELDDYSFSCYSQLEVNGSQHSLTCAFEDPDVNTTNLEFE |
| and 2 | ICGALVEVKCLNFRKLQEIYFIETKKFLLIGKSNICVKVGEKSLTCKKIDLT |
| | TIVKPEAPFDLSVVYREGANDFVVTFNTSHLQKKYVKVLMHDVAYRQEKDEN |
| | KWTHVNLSSTKLTLLQRKLQPAAMYEIKVRSIPDHYFKGFWSEWSPSYYFRT |
| | PEINNSSGEMD [symbol] DCDIEGKDGKQYESVLMVSIDQLLDSMKE |
| | IGSNCLNNEFNFFKRHICDANKEGMFLFRAARKLRQFLKMNSTGDFDLHLLK |
| | VSEGTTILLNCTGQVKGRKPAALGAAQPTKSLEENKSLKEQKKLNDLCFLKR |
| | LLQEIKTCWNKILMGTKEH [symbol] EPKSSDKTHTCPPCPAPEAAGG |
| | PSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALAAPIEKTISKAKG |
| | QPREPQVYTLPPSRDELTKNQVSLTCLVKGFYPSDIAVEWESNGQPENNYKT |
| | TPPVLDSDGSFFLYSKLTVDKSRWQQGNVFSCSVMHEALHNHYTQKSLSLSP |
| | GK |

P2994 IL-7/IL-7R

| | |
|---|---|
| Chains 1 | ESGYAQNGDLEDAELDDYSFSCYSQLEVNGSQHSLTCAFEDPDVNTTNLEFE |
| and 2 | ICGALVEVKCLNFRKLQEIYFIETAKFLLIGKSNICVKVGEKSLTCKKIDLT |
| | TIVKPEAPFDLSVVYREGANDFVVTFNTSHLQKKYVKVLMHDVAYRQEKDEN |
| | KWTHVNLSSTKLTLLQRKLQPAAMYEIKVRSIPDHYFKGFWSEWSPSYYFRT |
| | PEINNSSGEMD [symbol] DCDIEGKDGKQYESVLMVSIDQLLDSMKE |
| | IGSNCLNNEFNFFKRHICDANKEGMFLFRAARKLRQFLKMNSTGDFDLHLLK |
| | VSEGTTILLNCTGQVKGRKPAALGAAQPTKSLEENKSLKEQKKLNDLCFLKR |
| | LLQEIKTCWNKILMGTKEH [symbol] EPKSSDKTHTCPPCPAPEAAGG |
| | PSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALAAPIEKTISKAKG |
| | QPREPQVYTLPPSRDELTKNQVSLTCLVKGFYPSDIAVEWESNGQPENNYKT |
| | TPPVLDSDGSFFLYSKLTVDKSRWQQGNVFSCSVMHEALHNHYTQKSLSLSP |
| | GK |

P2995 IL-7/IL-7R

| | |
|---|---|
| Chains 1 | ESGYAQNGDLEDAELDDYSFSCYSQLEVNGSQHSLTCAFEDPDVNTTNLEFE |
| and 2 | ICGALVEVKCLNFRKLQEIYFIETKKFALIGKSNICVKVGEKSLTCKKIDLT |
| | TIVKPEAPFDLSVVYREGANDFVVTFNTSHLQKKYVKVLMHDVAYRQEKDEN |
| | KWTHVNLSSTKLTLLQRKLQPAAMYEIKVRSIPDHYFKGFWSEWSPSYYFRT |
| | PEINNSSGEMD [symbol] DCDIEGKDGKQYESVLMVSIDQLLDSMKE |
| | IGSNCLNNEFNFFKRHICDANKEGMFLFRAARKLRQFLKMNSTGDFDLHLLK |
| | VSEGTTILLNCTGQVKGRKPAALGAAQPTKSLEENKSLKEQKKLNDLCFLKR |
| | LLQEIKTCWNKILMGTKEH [symbol] EPKSSDKTHTCPPCPAPEAAGG |
| | PSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALAAPIEKTISKAKG |
| | QPREPQVYTLPPSRDELTKNQVSLTCLVKGFYPSDIAVEWESNGQPENNYKT |
| | TPPVLDSDGSFFLYSKLTVDKSRWQQGNVFSCSVMHEALHNHYTQKSLSLSP |
| | GK |

P2996 IL-7/IL-7R

| | |
|---|---|
| Chains 1 | ESGYAQNGDLEDAELDDYSFSCYSQLEVNGSQHSLTCAFEDPDVNTTNLEFE |
| and 2 | ICGALVEVKCLNFRKLQEIYFIETKKFLLIGKSNICVKVGEKSLTCKKIDLT |
| | TIVKPEAPFDLSVVYREGANDFVVTFNTSHLQKAYVKVLMHDVAYRQEKDEN |
| | KWTHVNLSSTKLTLLQRKLQPAAMYEIKVRSIPDHYFKGFWSEWSPSYYFRT |
| | PEINNSSGEMD [symbol] DCDIEGKDGKQYESVLMVSIDQLLDSMKE |
| | IGSNCLNNEFNFFKRHICDANKEGMFLFRAARKLRQFLKMNSTGDFDLHLLK |
| | VSEGTTILLNCTGQVKGRKPAALGAAQPTKSLEENKSLKEQKKLNDLCFLKR |
| | LLQEIKTCWNKILMGTKEH [symbol] EPKSSDKTHTCPPCPAPEAAGG |
| | PSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALAAPIEKTISKAKG |
| | QPREPQVYTLPPSRDELTKNQVSLTCLVKGFYPSDIAVEWESNGQPENNYKT |
| | TPPVLDSDGSFFLYSKLTVDKSRWQQGNVFSCSVMHEALHNHYTQKSLSLSP |
| | GK |

P2997 IL-21/IL-21R

| | |
|---|---|
| Chains 1 | CPDLVCYTDYLQTVICILEMWNLHPSTLTLTWQDQYEELKDEATSCSLHRSA |
| and 2 | HNATHATYTCHMDVFHFMADDIFSVNITDQSGNYSQECGSFLLAESIKPAPP |
| | FNVTVTFSGQYNISWRSDYEDPAFYMLKGKLQYELQYRNRGDPWAVSPRRKL |
| | ISVDSRSVSLLPLEFRKDSSYELQVRAGPMPGSSYQGTWSEWSDPVIFQTQS |
| | EELKE [symbol] QGQDRHMIRMRQLIDIVDQLKNYVNDLVPEFLPAPEDVE |
| | TNCEWSAFSCFQKAQLKSANTGNNERIINVSIKKLKRKPPSTNAGRRQKHRL |
| | TCPSCDSYEKKPPKEFLERFKSLLQKMIHQHLSSRTHGSEDS [symbol] |
| | EPKSSDKTHTCPPCPAPEAAGGPSVFLFPPKPKDTLMISRTPEVTCVVVDV |
| | SHEDPEVKFNWYVDGVEVHNAKTKPREEQYNSTYRVVSVLTVLHQDWLNGKE |
| | YKCKVSNKALAAPIEKTISKAKGQPREPQVYTLPPSRDELTKNQVSLTCLVK |
| | GFYPSDIAVEWESNGQPENNYKTTPPVLDSDGSFFLYSKLTVDKSRWQQGNV |
| | FSCSVMHEALHNHYTQKSLSLSPGK |

P2998 IL-21/IL-21R

| | |
|---|---|
| Chains 1 | CPDLVCYTDYLQTVICILEMWNLHPSTLTLTWQDQAEELKDEATSCSLHRSA |
| and 2 | HNATHATYTCHMDVFHFMADDIFSVNITDQSGNYSQECGSFLLAESIKPAPP |
| | FNVTVTFSGQYNISWRSDYEDPAFYMLKGKLQYELQYRNRGDPWAVSPRRKL |
| | ISVDSRSVSLLPLEFRKDSSYELQVRAGPMPGSSYQGTWSEWSDPVIFQTQS |
| | EELKE [symbol] QGQDRHMIRMRQLIDIVDQLKNYVNDLVPEFLPAPEDVE |
| | TNCEWSAFSCFQKAQLKSANTGNNERIINVSIKKLKRKPPSTNAGRRQKHRL |
| | TCPSCDSYEKKPPKEFLERFKSLLQKMIHQHLSSRTHGSEDS [symbol] |
| | EPKSSDKTHTCPPCPAPEAAGGPSVFLFPPKPKDTLMISRTPEVTCVVVDV |
| | SHEDPEVKFNWYVDGVEVHNAKTKPREEQYNSTYRVVSVLTVLHQDWLNGKE |
| | YKCKVSNKALAAPIEKTISKAKGQPREPQVYTLPPSRDELTKNQVSLTCLVK |
| | GFYPSDIAVEWESNGQPENNYKTTPPVLDSDGSFFLYSKLTVDKSRWQQGNV |
| | FSCSVMHEALHNHYTQKSLSLSPGK |

P2999 IL-21/IL-21R

| | |
|---|---|
| Chains 1 | CPDLVCYTDYLQTVICILEMWNLHPSTLTLTWQDQYEELKDEATSCSLHRSA |
| and 2 | HNATHATYTCHMDVFHFAADDIFSVNITDQSGNYSQECGSFLLAESIKPAPP |
| | FNVTVTFSGQYNISWRSDYEDPAFYMLKGKLQYELQYRNRGDPWAVSPRRKL |
| | ISVDSRSVSLLPLEFRKDSSYELQVRAGPMPGSSYQGTWSEWSDPVIFQTQS |
| | EELKE [symbol] QGQDRHMIRMRQLIDIVDQLKNYVNDLVPEFLPAPEDVE |
| | TNCEWSAFSCFQKAQLKSANTGNNERIINVSIKKLKRKPPSTNAGRRQKHRL |
| | TCPSCDSYEKKPPKEFLERFKSLLQKMIHQHLSSRTHGSEDS [symbol] |
| | EPKSSDKTHTCPPCPAPEAAGGPSVFLFPPKPKDTLMISRTPEVTCVVVDV |
| | SHEDPEVKFNWYVDGVEVHNAKTKPREEQYNSTYRVVSVLTVLHQDWLNGKE |
| | YKCKVSNKALAAPIEKTISKAKGQPREPQVYTLPPSRDELTKNQVSLTCLVK |
| | GFYPSDIAVEWESNGQPENNYKTTPPVLDSDGSFFLYSKLTVDKSRWQQGNV |
| | FSCSVMHEALHNHYTQKSLSLSPGK |

P3000 IL-21/IL-21R

| | |
|---|---|
| Chains 1 | CPDLVCYTDYLQTVICILEMWNLHPSTLTLTWQDQYEELKDEATSCSLHRSA |
| and 2 | HNATHATYTCHMDVFHFMAADIFSVNITDQSGNYSQECGSFLLAESIKPAPP |
| | FNVTVTFSGQYNISWRSDYEDPAFYMLKGKLQYELQYRNRGDPWAVSPRRKL |
| | ISVDSRSVSLLPLEFRKDSSYELQVRAGPMPGSSYQGTWSEWSDPVIFQTQS |
| | EELKE [symbol] QGQDRHMIRMRQLIDIVDQLKNYVNDLVPEFLPAPEDVE |
| | TNCEWSAFSCFQKAQLKSANTGNNERIINVSIKKLKRKPPSTNAGRRQKHRL |
| | TCPSCDSYEKKPPKEFLERFKSLLQKMIHQHLSSRTHGSEDS [symbol] |
| | EPKSSDKTHTCPPCPAPEAAGGPSVFLFPPKPKDTLMISRTPEVTCVVVDV |
| | SHEDPEVKFNWYVDGVEVHNAKTKPREEQYNSTYRVVSVLTVLHQDWLNGKE |
| | YKCKVSNKALAAPIEKTISKAKGQPREPQVYTLPPSRDELTKNQVSLTCLVK |
| | GFYPSDIAVEWESNGQPENNYKTTPPVLDSDGSFFLYSKLTVDKSRWQQGNV |
| | FSCSVMHEALHNHYTQKSLSLSPGK |

P3001 IL-21/IL-21R

| | |
|---|---|
| Chains 1 | CPDLVCYTDYLQTVICILEMWNLHPSTLTLTWQDQYEELKDEATSCSLHRSA |
| and 2 | HNATHATYTCHMDVFHFMADAIFSVNITDQSGNYSQECGSFLLAESIKPAPP |
| | FNVTVTFSGQYNISWRSDYEDPAFYMLKGKLQYELQYRNRGDPWAVSPRRKL |
| | ISVDSRSVSLLPLEFRKDSSYELQVRAGPMPGSSYQGTWSEWSDPVIFQTQS |
| | EELKE [symbol] QGQDRHMIRMRQLIDIVDQLKNYVNDLVPEFLPAPEDVE |
| | TNCEWSAFSCFQKAQLKSANTGNNERIINVSIKKLKRKPPSTNAGRRQKHRL |
| | TCPSCDSYEKKPPKEFLERFKSLLQKMIHQHLSSRTHGSEDS [symbol] |
| | EPKSSDKTHTCPPCPAPEAAGGPSVFLFPPKPKDTLMISRTPEVTCVVVDV |
| | SHEDPEVKFNWYVDGVEVHNAKTKPREEQYNSTYRVVSVLTVLHQDWLNGKE |
| | YKCKVSNKALAAPIEKTISKAKGQPREPQVYTLPPSRDELTKNQVSLTCLVK |
| | GFYPSDIAVEWESNGQPENNYKTTPPVLDSDGSFFLYSKLTVDKSRWQQGNV |
| | FSCSVMHEALHNHYTQKSLSLSPGK |

P3002 IL-21/IL-21R

| | |
|---|---|
| Chains 1 | CPDLVCYTDYLQTVICILEMWNLHPSTLTLTWQDQYEELKDEATSCSLHRSA |
| and 2 | HNATHATYTCHMDVFHFMADDIFSVNITDQSGNYSQECGSFLLAESIKPAPP |
| | FNVTVTFSGQYNISWRSDYEDPAFAMLKGKLQYELQYRNRGDPWAVSPRRKL |
| | ISVDSRSVSLLPLEFRKDSSYELQVRAGPMPGSSYQGTWSEWSDPVIFQTQS |
| | EELKE [symbol] QGQDRHMIRMRQLIDIVDQLKNYVNDLVPEFLPAPEDVE |
| | TNCEWSAFSCFQKAQLKSANTGNNERIINVSIKKLKRKPPSTNAGRRQKHRL |
| | TCPSCDSYEKKPPKEFLERFKSLLQKMIHQHLSSRTHGSEDS [symbol] |
| | EPKSSDKTHTCPPCPAPEAAGGPSVFLFPPKPKDTLMISRTPEVTCVVVDV |
| | SHEDPEVKFNWYVDGVEVHNAKTKPREEQYNSTYRWSVLTVLHQDWLNGKE |
| | YKCKVSNKALAAPIEKTISKAKGQPREPQVYTLPPSRDELTKNQVSLTCLVK |
| | GFYPSDIAVEWESNGQPENNYKTTPPVLDSDGSFFLYSKLTVDKSRWQQGNV |
| | FSCSVMHEALHNHYTQKSLSLSPGK |

P3003 IL-21/IL-21R

| | |
|---|---|
| Chains 1 | CPDLVCYTDYLQTVICILEMWNLHPSTLTLTWQDQAEELKDEATSCSLHRSA |
| and 2 | HNATHATYTCHMDVFHFMADDIFSVNITDQSGNYSQECGSFLLAESIKPAPP |
| | FNVTVTFSGQYNISWRSDYEDPAFAMLKGKLQYELQYRNRGDPWAVSPRRKL |
| | ISVDSRSVSLLPLEFRKDSSYELQVRAGPMPGSSYQGTWSEWSDPVIFQTQS |
| | EELKE [symbol] QGQDRHMIRMRQLIDIVDQLKNYVNDLVPEFLPAPEDVE |
| | TNCEWSAFSCFQKAQLKSANTGNNERIINVSIKKLKRKPPSTNAGRRQKHRL |
| | TCPSCDSYEKKPPKEFLERFKSLLQKMIHQHLSSRTHGSEDS [symbol] |
| | EPKSSDKTHTCPPCPAPEAAGGPSVFLFPPKPKDTLMISRTPEVTCVVVDV |
| | SHEDPEVKFNWYVDGVEVHNAKTKPREEQYNSTYRWSVLTVLHQDWLNGKE |
| | YKCKVSNKALAAPIEKTISKAKGQPREPQVYTLPPSRDELTKNQVSLTCLVK |
| | GFYPSDIAVEWESNGQPENNYKTTPPVLDSDGSFFLYSKLTVDKSRWQQGNV |
| | FSCSVMHEALHNHYTQKSLSLSPGK |

P3004 IL-21/IL-21R

| | |
|---|---|
| Chains 1 | CPDLVCYTDYLQTVICILEMWNLHPSTLTLTWQDQYEELKDEATSCSLHRSA |
| and 2 | HNATHATYTCHMDVFHFMADDIFSVNITDQSGNYSQECGSFLLAESIKPAPP |
| | FNVTVTFSGQYNISWRSDYEDPAFYMLKGKLQYELQYRNRGDPWAVSPRRKL |
| | ISVDSRSVSLLPLEFRKDSSYELQVRAGPMPGSSYQGTWSEWSDPVIFQTQS |
| | EELKE [symbol] QGQDRHMIRMRQLIDIVDQLKNYVNDLVPEFLPAP |
| | EDVETNCEWSAFSCFQKAQLKSANTGNNERIINVSIKKLKRKPPSTNAGRRQ |
| | KHRLTCPSCDSYEKKPPKEFLERFKSLLQKMIHQHLSSRTHGSEDS [symbol] |
| | EPKSSDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWL |
| | NGKEYKCKVSNKALAAPIEKTISKAKGQPREPQVYTLPPSRDELTKNQVSLT |
| | CLVKGFYPSDIAVEWESNGQPENNYKTTPPVLDSDGSFFLYSKLTVDKSRWQ |
| | QGNVFSCSVMHEALHNHYTQKSLSLSPGK |

P3005 IL-21/IL-21R

| | |
|---|---|
| Chains 1 | CPDLVCYTDYLQTVICILEMWNLHPSTLTLTWQDQAEELKDEATSCSLHRSA |
| and 2 | HNATHATYTCHMDVFHFMADDIFSVNITDQSGNYSQECGSFLLAESIKPAPP |
| | FNVTVTFSGQYNISWRSDYEDPAFYMLKGKLQYELQYRNRGDPWAVSPRRKL |
| | ISVDSRSVSLLPLEFRKDSSYELQVRAGPMPGSSYQGTWSEWSDPVIFQTQS |
| | EELKE [symbol] QGQDRHMIRMRQLIDIVDQLKNYVNDLVPEFLPAP |
| | EDVETNCEWSAFSCFQKAQLKSANTGNNERIINVSIKKLKRKPPSTNAGRRQ |
| | KHRLTCPSCDSYEKKPPKEFLERFKSLLQKMIHQHLSSRTHGSEDS [symbol] |
| | EPKSSDKTHTCPPCPAPEAAGGPSVFLFPPKPKDTLMISRTPEVTCV |
| | VVDVSHEDPEVKFNWYVDGVEVHNAKTKPREEQYNSTYVVWSVLTVLHQDWL |
| | NGKEYKCKVSNKALAAPIEKTISKAKGQPREPQVYTLPPSRDELTKNQVSLT |
| | CLVKGFYPSDIAVEWESNGQPENNYKTTPPVLDSDGSFFLYSKLTVDKSRWQ |
| | QGNVFSCSVMHEALHNHYTQKSLSLSPGK |

P3006 IL-21/IL-21R

| | |
|---|---|
| Chains 1 | CPDLVCYTDYLQTVICILEMWNLHPSTLTLTWQDQYEELKDEATSCSLHRSA |
| and 2 | HNATHATYTCHMDVFHFAADDIFSVNITDQSGNYSQECGSFLLAESIKPAPP |
| | FNVTVTFSGQYNISWRSDYEDPAFYMLKGKLQYELQYRNRGDPWAVSPRRKL |
| | ISVDSRSVSLLPLEFRKDSSYELQVRAGPMPGSSYQGTWSEWSDPVIFQTQS |
| | EELKE [symbol] QGQDRHMIRMRQLIDIVDQLKNYVNDLVPEFLPAP |
| | EDVETNCEWSAFSCFQKAQLKSANTGNNERIINVSIKKLKRKPPSTNAGRRQ |
| | KHRLTCPSCDSYEKKPPKEFLERFKSLLQKMIHQHLSSRTHGSEDS [symbol] |
| | EPKSSDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWL |
| | NGKEYKCKVSNKALAAPIEKTISKAKGQPREPQVYTLPPSRDELTKNQVSLT |
| | CLVKGFYPSDIAVEWESNGQPENNYKTTPPVLDSDGSFFLYSKLTVDKSRWQ |
| | QGNVFSCSVMHEALHNHYTQKSLSLSPGK |

P3007 IL-21/IL-21R

| | |
|---|---|
| Chains 1 | CPDLVCYTDYLQTVICILEMWNLHPSTLTLTWQDQYEELKDEATSCSLHRSA |
| and 2 | HNATHATYTCHMDVFHFMAADIFSVNITDQSGNYSQECGSFLLAESIKPAPP |
| | FNVTVTFSGQYNISWRSDYEDPAFYMLKGKLQYELQYRNRGDPWAVSPRRKL |
| | ISVDSRSVSLLPLEFRKDSSYELQVRAGPMPGSSYQGTWSEWSDPVIFQTQS |
| | EELKE [symbol] QGQDRHMIRMRQLIDIVDQLKNYVNDLVPEFLPAP |
| | EDVETNCEWSAFSCFQKAQLKSANTGNNERIINVSIKKLKRKPPSTNAGRRQ |
| | KHRLTCPSCDSYEKKPPKEFLERFKSLLQKMIHQHLSSRTHGSEDS [symbol] |
| | EPKSSDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWL |
| | NGKEYKCKVSNKALAAPIEKTISKAKGQPREPQVYTLPPSRDELTKNQVSLT |
| | CLVKGFYPSDIAVEWESNGQPENNYKTTPPVLDSDGSFFLYSKLTVDKSRWQ |
| | QGNVFSCSVMHEALHNHYTQKSLSLSPGK |

P3008 IL-21/IL-21R

| | |
|---|---|
| Chains 1 | CPDLVCYTDYLQTVICILEMWNLHPSTLTLTWQDQYEELKDEATSCSLHRSA |
| and 2 | HNATHATYTCHMDVFHFMADAIFSVNITDQSGNYSQECGSFLLAESIKPAPP |
| | FNVTVTFSGQYNISWRSDYEDPAFYMLKGKLQYELQYRNRGDPWAVSPRRKL |
| | ISVDSRSVSLLPLEFRKDSSYELQVRAGPMPGSSYQGTWSEWSDPVIFQTQS |
| | EELKE [symbol] QGQDRHMIRMRQLIDIVDQLKNYVNDLVPEFLPAP |
| | EDVETNCEWSAFSCFQKAQLKSANTGNNERIINVSIKKLKRKPPSTNAGRRQ |
| | KHRLTCPSCDSYEKKPPKEFLERFKSLLQKMIHQHLSSRTHGSEDS [symbol] |
| | EPKSSDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWL |
| | NGKEYKCKVSNKALAAPIEKTISKAKGQPREPQVYTLPPSRDELTKNQVSLT |
| | CLVKGFYPSDIAVEWESNGQPENNYKTTPPVLDSDGSFFLYSKLTVDKSRWQ |
| | QGNVFSCSVMHEALHNHYTQKSLSLSPGK |

P3009 IL-21/IL-21R

| | |
|---|---|
| Chains 1 | CPDLVCYTDYLQTVICILEMWNLHPSTLTLTWQDQYEELKDEATSCSLHRSA |
| and 2 | HNATHATYTCHMDVFHFMADDIFSVNITDQSGNYSQECGSFLLAESIKPAPP |
| | FNVTVTFSGQYNISWRSDYEDPAFAMLKGKLQYELQYRNRGDPWAVSPRRKL |
| | ISVDSRSVSLLPLEFRKDSSYELQVRAGPMPGSSYQGTWSEWSDPVIFQTQS |
| | EELKE [symbol] QGQDRHMIRMRQLIDIVDQLKNYVNDLVPEFLPAP |
| | EDVETNCEWSAFSCFQKAQLKSANTGNNERIINVSIKKLKRKPPSTNAGRRQ |
| | KHRLTCPSCDSYEKKPPKEFLERFKSLLQKMIHQHLSSRTHGSEDS [symbol] |
| | EPKSSDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWL |
| | NGKEYKCKVSNKALAAPIEKTISKAKGQPREPQVYTLPPSRDELTKNQVSLT |
| | CLVKGFYPSDIAVEWESNGQPENNYKTTPPVLDSDGSFFLYSKLTVDKSRWQ |
| | QGNVFSCSVMHEALHNHYTQKSLSLSPGK |

P3010 IL-21/IL-21R

| | |
|---|---|
| Chains 1 | CPDLVCYTDYLQTVICILEMWNLHPSTLTLTWQDQAEELKDEATSCSLHRSA |
| and 2 | HNATHATYTCHMDVFHFMADDIFSVNITDQSGNYSQECGSFLLAESIKPAPP |
| | FNVTVTFSGQYNISWRSDYEDPAFAMLKGKLQYELQYRNRGDPWAVSPRRKL |
| | ISVDSRSVSLLPLEFRKDSSYELQVRAGPMPGSSYQGTWSEWSDPVIFQTQS |
| | EELKE [symbol] QGQDRHMIRMRQLIDIVDQLKNYVNDLVPEFLPAP |
| | EDVETNCEWSAFSCFQKAQLKSANTGNNERIINVSIKKLKRKPPSTNAGRRQ |
| | KHRLTCPSCDSYEKKPPKEFLERFKSLLQKMIHQHLSSRTHGSEDS [symbol] |
| | EPKSSDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWL |
| | NGKEYKCKVSNKALAAPIEKTISKAKGQPREPQVYTLPPSRDELTKNQVSLT |
| | CLVKGFYPSDIAVEWESNGQPENNYKTTPPVLDSDGSFFLYSKLTVDKSRWQ |
| | QGNVFSCSVMHEALHNHYTQKSLSLSPGK |

P3011 IFN&/IFNAR

| | |
|---|---|
| Chains 1 | ESCTFKISLRNFRSILSWELKNHSIVPTHYTLLYTIMSKPEDLKVVKNCANT |
| and 2 | TRSFCDLTDEWRSTHEAYVTVLEGFSGNTTLFSCSHNFWLAIDMSFEPPEFE |
| | IVGFTNHINVMVKFPSIVEEELQFDLSLVIEEQSEGIVKKHKPEIKGNMSGN |
| | FTYIIDKLIPNTNYCVSVYLEHSDEQAVIKSPLKCTLLPP [symbol] CDLP |
| | QTHSLGSRRTLMLLAQMRRISLFSCLKDRHDFGFPQEEFGNQFQKAETIPVL |
| | HEMIQQIFNLFSTKDSSAAWDETLLDKFYTELYQQLNDLEACVIQGVGVTET |
| | PLMKEDSILAVRKYFQRITLYLKEKKYSPCAWEVVRAEIMRSFSLSTNLQES |
| | LRSKE [symbol] EPKSSDKTHTCPPCPAPEAAGGPSVFLFPPKPKDTL |
| | MISRTPEVTCVVVDVSHEDPEVKFNWYVDGVEVHNAKTKPREEQYNSTYRVV |
| | SVLTVLHQDWLNGKEYKCKVSNKALAAPIEKTISKAKGQPREPQVYTLPPSR |
| | DELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |

P3012 IFNα/IFNAR

| | |
|---|---|
| Chains 1 | ESCTFKISLRNFRSILSWELKNHSIVPTHYTLLYTIASKPEDLKVVKNCANT |
| and 2 | TRSFCDLTDEWRSTHEAYVTVLEGFSGNTTLFSCSHNFWLAIDMSFEPPEFE |
| | IVGFTNHINVMVKFPSIVEEELQFDLSLVIEEQSEGIVKKHKPEIKGNMSGN |
| | FTYIIDKLIPNTNYCVSVYLEHSDEQAVIKSPLKCTLLPP [symbol] CDLP |
| | QTHSLGSRRTLMLLAQMRRISLFSCLKDRHDFGFPQEEFGNQFQKAETIPVL |
| | HEMIQQIFNLFSTKDSSAAWDETLLDKFYTELYQQLNDLEACVIQGVGVTET |
| | PLMKEDSILAVRKYFQRITLYLKEKKYSPCAWEVVRAEIMRSFSLSTNLQES |
| | LRSKE [symbol] EPKSSDKTHTCPPCPAPEAAGGPSVFLFPPKPKDTL |
| | MISRTPEVTCVVVDVSHEDPEVKFNWYVDGVEVHNAKTKPREEQYNSTYRVV |
| | SVLTVLHQDWLNGKEYKCKVSNKALAAPIEKTISKAKGQPREPQVYTLPPSR |
| | DELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |

P3013 IFNα/IFNAR

| | |
|---|---|
| Chains 1 | ESCTFKISLRNFRSILSWELKNHSIVPTHYTLLYTIMSKPEDLKVVKNCANT |
| and 2 | TRSFCDLTDEWRSTHAAYVTVLEGFSGNTTLFSCSHNFWLAIDMSFEPPEFE |
| | IVGFTNHINVMVKFPSIVEEELQFDLSLVIEEQSEGIVKKHKPEIKGNMSGN |
| | FTYIIDKLIPNTNYCVSVYLEHSDEQAVIKSPLKCTLLPP [symbol] CDLP |
| | QTHSLGSRRTLMLLAQMRRISLFSCLKDRHDFGFPQEEFGNQFQKAETIPVL |
| | HEMIQQIFNLFSTKDSSAAWDETLLDKFYTELYQQLNDLEACVIQGVGVTET |
| | PLMKEDSILAVRKYFQRITLYLKEKKYSPCAWEVVRAEIMRSFSLSTNLQES |
| | LRSKE [symbol] EPKSSDKTHTCPPCPAPEAAGGPSVFLFPPKPKDTL |
| | MISRTPEVTCVVVDVSHEDPEVKFNWYVDGVEVHNAKTKPREEQYNSTYRVV |
| | SVLTVLHQDWLNGKEYKCKVSNKALAAPIEKTISKAKGQPREPQVYTLPPSR |
| | DELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |

P3014 IFNα/IFNAR

| | |
|---|---|
| Chains 1 | ESCTFKISLRNFRSILSWELKNHSIVPTHYTLLYTIVSKPEDLKVVKNCANT |
| and 2 | TRSFCDLTDEWRSTHAAYVTVLEGFSGNTTLFSCSHNFWLAIDMSFEPPEFE |
| | IVGFTNHINVMVKFPSIVEEELQFDLSLVIEEQSEGIVKKHKPEIKGNMSGN |
| | FTYIIDKLIPNTNYCVSVYLEHSDEQAVIKSPLKCTLLPP [symbol] CDLP |
| | QTHSLGSRRTLMLLAQMRRISLFSCLKDRHDFGFPQEEFGNQFQKAETIPVL |
| | HEMIQQIFNLFSTKDSSAAWDETLLDKFYTELYQQLNDLEACVIQGVGVTET |
| | PLMKEDSILAVRKYFQRITLYLKEKKYSPCAWEVVRAEIMRSFSLSTNLQES |
| | LRSKE [symbol] EPKSSDKTHTCPPCPAPEAAGGPSVFLFPPKPKDTL |
| | MISRTPEVTCVVVDVSHEDPEVKFNWYVDGVEVHNAKTKPREEQYNSTYRVV |
| | SVLTVLHQDWLNGKEYKCKVSNKALAAPIEKTISKAKGQPREPQVYTLPPSR |
| | DELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |

P3015 IFNα/IFNAR

| | |
|---|---|
| Chains 1 | EPKSSDKTHTCPPCPAPEAAGGPSVFLFPPKPKDTLMISRTPEVTCVVVDVS |
| and 2 | HEDPEVKFNWYVDGVEVHNAKTKPREEQYNSTYRVVSVLTVLHQDWLNGKEY |
| | KCKVSNKALAAPIEKTISKAKGQPREPQVYTLPPSRDELTKNQVSLTCLVKG |
| | FYPSDIAVEWESNGQPENNYKTTPPVLDSDGSFFLYSKLTVDKSRWQQGNVF |
| | SCSVMHEALHNHYTQKSLSLSPGK [symbol] SCTFKISLRNFR |
| | SILSWELKNHSIVPTHYTLLYTIVSKPEDLKVVKNCANTTRSFCDLTDEWRS |
| | THAAYVTVLEGFSGNTTLFSCSHNFWLAIDMSFEPPEFEIVGFTNHINVMVK |
| | FPSIVEEELQFDLSLVIEEQSEGIVKKHKPEIKGNMSGNFTYIIDKLIPNTN |
| | YCVSVYLEHSDEQAVIKSPLKCTLLPP [symbol] CDLPQTHSLGSRRTLML |
| | LAQMRRISLFSCLKDRHDFGFPQEEFGNQFQKAETIPVLHEMIQQIFNLFST |
| | KDSSAAWDETLLDKFYTELYQQLNDLEACVIQGVGVTETPLMKEDSILAVRK |
| | YFQRITLYLKEKKYSPCAWEVVRAEIMRSFSLSTNLQESLRSKE |

P3016 IFNα/IFNAR

| | |
|---|---|
| Chains 1 | ESCTFKISLRNFRSILSWELKNHSIVPTHYTLLYTIMSKPEDLKVVKNCANT |
| and 2 | TRSFCDLTDEWRSTHEAYVTVLEGFSGNTTLFSCSHNFWLAIDMSFEPPEFE |
| | IVGFTNHINVMVKFPSIVEEELQFDLSLVIEEQSEGIVKKHKPEIKGNMSGN |
| | FTYIIDKLIPNTNYCVSVYLEHSDEQAVIKSPLKCTLLPP [symbol] |
| | CDLPQTHSLGSRRTLMLLAQMRRISLFSCLKDRHDFGFPQEEFGNQFQKAET |
| | IPVLHEMIQQIFNLFSTKDSSAAWDETLLDKFYTELYQQLNDLEACVIQGVG |
| | VTETPLMKEDSILAVRKYFQRITLYLKEKKYSPCAWEVVRAEIMRSFSLSTN |
| | LQESLRSKE [symbol] EPKSSDKTHTCPPCPAPEAAGGPSVFLFPPKP |
| | KDTLMISRTPEVTCVVVDVSHEDPEVKFNWYVDGVEVHNAKTKPREEQYNST |
| | YRVVSVLTVLHQDWLNGKEYKCKVSNKALAAPIEKTISKAKGQPREPQVYTL |
| | PPSRDELTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |

P3017 IFNα/IFNAR

| | |
|---|---|
| Chains 1 | ESCTFKISLRNFRSILSWELKNHSIVPTHYTLLYTIASKPEDLKVVKNCANT |
| and 2 | TRSFCDLTDEWRSTHEAYVTVLEGFSGNTTLFSCSHNFWLAIDMSFEPPEFE |
| | IVGFTNHINVMVKFPSIVEEELQFDLSLVIEEQSEGIVKKHKPEIKGNMSGN |
| | FTYIIDKLIPNTNYCVSVYLEHSDEQAVIKSPLKCTLLPP [symbol] |
| | CDLPQTHSLGSRRTLMLLAQMRRISLFSCLKDRHDFGFPQEEFGNQFQKAET |
| | IPVLHEMIQQIFNLFSTKDSSAAWDETLLDKFYTELYQQLNDLEACVIQGVG |
| | VTETPLMKEDSILAVRKYFQRITLYLKEKKYSPCAWEVVRAEIMRSFSLSTN |
| | LQESLRSKE [symbol] EPKSSDKTHTCPPCPAPEAAGGPSVFLFPPKP |
| | KDTLMISRTPEVTCVVVDVSHEDPEVKFNWYVDGVEVHNAKTKPREEQYNST |
| | YRVVSVLTVLHQDWLNGKEYKCKVSNKALAAPIEKTISKAKGQPREPQVYTL |
| | PPSRDELTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |

P3018 IFNα/IFNAR

| | |
|---|---|
| Chains 1 | ESCTFKISLRNFRSILSWELKNHSIVPTHYTLLYTIMSKPEDLKVVKNCANT |
| and 2 | TRSFCDLTDEWRSTHAAYVTVLEGFSGNTTLFSCSHNFWLAIDMSFEPPEFE |
| | IVGFTNHINVMVKFPSIVEEELQFDLSLVIEEQSEGIVKKHKPEIKGNMSGN |
| | FTYIIDKLIPNTNYCVSVYLEHSDEQAVIKSPLKCTLLPP [symbol] |
| | CDLPQTHSLGSRRTLMLLAQMRRISLFSCLKDRHDFGFPQEEFGNQFQKAET |
| | IPVLHEMIQQIFNLFSTKDSSAAWDETLLDKFYTELYQQLNDLEACVIQGVG |
| | VTETPLMKEDSILAVRKYFQRITLYLKEKKYSPCAWEVVRAEIMRSFSLSTN |
| | LQESLRSKE [symbol] EPKSSDKTHTCPPCPAPEAAGGPSVFLFPPKP |
| | KDTLMISRTPEVTCVVVDVSHEDPEVKFNWYVDGVEVHNAKTKPREEQYNST |
| | YRWSVLTVLHQDWLNGKEYKCKVSNKALAAPIEKTISKAKGQPREPQVYTL |
| | PPSRDELTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |

P3019 IFNα/IFNAR

| | |
|---|---|
| Chains 1 | ESCTFKISLRNFRSILSWELKNHSIVPTHYTLLYTIVSKPEDLKVVKNCANT |
| and 2 | TRSFCDLTDEWRSTHAAYVTVLEGFSGNTTLFSCSHNFWLAIDMSFEPPEFE |
| | IVGFTNHINVMVKFPSIVEEELQFDLSLVIEEQSEGIVKKHKPEIKGNMSGN |
| | FTYIIDKLIPNTNYCVSVYLEHSDEQAVIKSPLKCTLLPP [symbol] |
| | CDLPQTHSLGSRRTLMLLAQMRRISLFSCLKDRHDFGFPQEEFGNQFQKAET |
| | IPVLHEMIQQIFNLFSTKDSSAAWDETLLDKFYTELYQQLNDLEACVIQGVG |
| | VTETPLMKEDSILAVRKYFQRITLYLKEKKYSPCAWEVVRAEIMRSFSLSTN |
| | LQESLRSKE [symbol] EPKSSDKTHTCPPCPAPEAAGGPSVFLFPPKP |
| | KDTLMISRTPEVTCVVVDVSHEDPEVKFNWYVDGVEVHNAKTKPREEQYNST |
| | YRVVSVLTVLHQDWLNGKEYKCKVSNKALAAPIEKTISKAKGQPREPQVYTL |
| | PPSRDELTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |

P3020 IFNα/IFNAR

| | |
|---|---|
| Chains 1 | EPKSSDKTHTCPPCPAPEAAGGPSVFLFPPKPKDTLMISRTPEVTCVVVDVS |
| and 2 | HEDPEVKFNWYVDGVEVHNAKTKPREEQYNSTYRVVSVLTVLHQDWLNGKEY |
| | KCKVSNKALAAPIEKTISKAKGQPREPQVYTLPPSRDELTKNQVSLTCLVKG |
| | FYPSDIAVEWESNGQPENNYKTTPPVLDSDGSFFLYSKLTVDKSRWQQGNVF |
| | SCSVMHEALHNHYTQKSLSLSPGK [symbol] ESCTFKISLRNFR |
| | SILSWELKNHSIVPTHYTLLYTIVSKPEDLKVVKNCANTTRSFCDLTDEWRS |
| | THAAYVTVLEGFSGNTTLFSCSHNFWLAIDMSFEPPEFEIVGFTNHINVMVK |
| | FPSIVEEELQFDLSLVIEEQSEGIVKKHKPEIKGNMSGNFTYIIDKLIPNTN |
| | YCVSVYLEHSDEQAVIKSPLKCTLLPP [symbol] CDLPQTHSLGSRR |
| | TLMLLAQMRRISLFSCLKDRHDFGFPQEEFGNQFQKAETIPVLHEMIQQIFN |
| | LFSTKDSSAAWDETLLDKFYTELYQQLNDLEACVIQGVGVTETPLMKEDSIL |
| | AVRKYFQRITLYLKEKKYSPCAWEVVRAEIMRSFSLSTNLQESLRSKE |

Thus, in certain embodiments, an activatable proprotein homodimer comprises a first polypeptide and a second polypeptide that comprises, consists, or consists essentially of an amino acid sequence that is at least 80, 85, 90, 95, 98, or 100% identical to a sequence selected from Table S15. In certain embodiments, the protease cleavage site of any one or more of the foregoing sequences (from Table S15) is substituted with a human protease cleavage site, that is, a cleavage site cleavable by a human protease, for example, a human protease expressed in a cancer tissue or cancer cell (see, for example, Table L2 for exemplary cleavable linkers).

Methods of Use and Pharmaceutical Compositions

Certain embodiments include methods of treating, ameliorating the symptoms of, and/or reducing the progression of, a disease or condition in a subject in need thereof, comprising administering to the subject at least one activatable proprotein homodimer, as described herein. Also included are methods of enhancing an immune response in a subject comprising administering to the subject at least one activatable proprotein homodimer, as described herein. In particular embodiments, the disease is selected from one or more of a cancer, a viral infection, and an immune disorder.

In some embodiments, following administration, the activatable proprotein is activated through protease cleavage in a cell or tissue, which exposes the binding site of the cytokine protein that binds to its wild-type cognate receptor present on the surface of the immune cell in vitro or in vivo, and thereby generates an activated protein. For instance, in some embodiments, the activated protein binds via the IL-7 protein to IL-7Rα/λc on the surface of the immune cell; or the activated protein binds via the IL-21 protein to IL-21R/λc on the surface of the immune cell; or the activated protein binds via the type I IFN protein to IFNAR1/IFNAR2 on the surface of the immune cell; or the activated protein binds via the type II IFN protein to IFNGR1/IFNGR2 on the surface of the immune cell; or the activated protein binds via the type III IFN protein to IL10Rβ/IFN-λR1 on the surface of the immune cell. Typically, the activated protein has at least one immune-stimulating activity, for example, by binding to its cognate receptor present on the surface of an immune cell in vivo, and thereby stimulating the immune cell. In particular embodiments, the immune cell is selected from one or more of a T cell, a B cell, a natural killer cell, a monocyte, and a macrophage.

In some embodiments, administration and activation of the activatable proprotein homodimer, to generate an activated protein, increases an immune response in the subject by about or at least about 5, 10, 15, 20, 25, 30, 35, 40, 45, 50, 60, 70, 80, 90, 100, 200, 300, 400, 500, 600, 700, 800, 900, 1000, 2000/o or more, relative to a control. In some instances, the immune response is an anti-cancer or anti-viral immune response. In some embodiments, administration and activation of the activatable proprotein homodimer, to generate an activated protein, increases cell-killing in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control. In some embodiments, wherein the cell-killing is cancer cell-killing or virally-infected cell-killing.

In some embodiments, the disease is a cancer, that is, the subject in need thereof has or is suspected of having a cancer. Certain embodiments thus include methods of treating, ameliorating the symptoms of, or inhibiting the progression of, a cancer in a subject in need thereof, comprising administering to the subject at least one activatable proprotein, as described herein. In particular embodiments, the cancer is a primary cancer or a metastatic cancer. In specific embodiments, the cancer is selected from one or more of melanoma (optionally metastatic melanoma), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer

In some embodiments, as noted above, the cancer is a metastatic cancer. Further to the above cancers, exemplary metastatic cancers include, without limitation, bladder cancers which have metastasized to the bone, liver, and/or lungs; breast cancers which have metastasized to the bone, brain, liver, and/or lungs; colorectal cancers which have metastasized to the liver, lungs, and/or peritoneum; kidney cancers which have metastasized to the adrenal glands, bone, brain, liver, and/or lungs; lung cancers which have metastasized to the adrenal glands, bone, brain, liver, and/or other lung sites; melanomas which have metastasized to the bone, brain, liver, lung, and/or skin/muscle; ovarian cancers which have metastasized to the liver, lung, and/or peritoneum; pancreatic cancers which have metastasized to the liver, lung, and/or peritoneum; prostate cancers which have metastasized to the adrenal glands, bone, liver, and/or lungs; stomach cancers which have metastasized to the liver, lung, and/or peritoneum; thyroid cancers which have metastasized to the bone, liver, and/or lungs; and uterine cancers which have metastasized to the bone, liver, lung, peritoneum, and/or vagina; among others.

The methods for treating cancers can be combined with other therapeutic modalities. For example, a combination therapy described herein can be administered to a subject before, during, or after other therapeutic interventions, including symptomatic care, radiotherapy, surgery, transplantation, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

Certain embodiments thus include combination therapies for treating cancers, including methods of treating ameliorating the symptoms of, or inhibiting the progression of, a cancer in a subject in need thereof, comprising administering to the subject at least one activatable proprotein described herein in combination with at least one additional agent, for example, a chemotherapeutic agent, a hormonal therapeutic agent, and/or a kinase inhibitor. In some embodiments, administering the at least one activatable proprotein enhances the susceptibility of the cancer to the additional agent (for example, chemotherapeutic agent, hormonal therapeutic agent, and or kinase inhibitor) by about or at least about 5, 10, 15, 20, 25, 30, 35, 40, 45, 50, 60, 70, 80, 90, 100, 200, 300, 400, 500, 600, 700, 800, 900, 1000, 2000% or more relative to the additional agent alone.

Certain combination therapies employ one or more chemotherapeutic agents, for example, small molecule chemotherapeutic agents. Non-limiting examples of chemotherapeutic agents include alkylating agents, anti-metabolites, cytotoxic antibiotics, topoisomerase inhibitors (type I or type II), an anti-microtubule agents, among others.

Examples of alkylating agents include nitrogen mustards (e.g., mechlorethamine, cyclophosphamide, mustine, melphalan, chlorambucil, ifosfamide, and busulfan), nitrosoureas (e.g., N-Nitroso-N-methylurea (MNU), carmustine (BCNU), lomustine (CCNU), semustine (MeCCNU), fotemustine, and streptozotocin), tetrazines (e.g., dacarbazine, mitozolomide, and temozolomide), aziridines (e.g., thiotepa, mytomycin, and diaziquone (AZQ)), cisplatins and derivatives thereof (e.g., carboplatin and oxaliplatin), and non-classical alkylating agents (optionally procarbazine and hexamethylmelamine).

Examples of anti-metabolites include anti-folates (e.g., methotrexate and pemetrexed), fluoropyrimidines (e.g., 5-fluorouracil and capecitabine), deoxynucleoside analogues (e.g., ancitabine, enocitabine, cytarabine, gemcitabine, decitabine, azacitidine, fludarabine, nelarabine, cladribine, clofarabine, fludarabine, and pentostatin), and thiopurines (e.g., thioguanine and mercaptopurine);

Examples of cytotoxic antibiotics include anthracyclines (e.g., doxorubicin, daunorubicin, epirubicin, idarubicin, pirarubicin, aclarubicin, and mitoxantrone), bleomycins, mitomycin C, mitoxantrone, and actinomycin. Examples of topoisomerase inhibitors include camptothecin, irinotecan, topotecan, etoposide, doxorubicin, mitoxantrone, teniposide, novobiocin, merbarone, and aclarubicin.

Examples of anti-microtubule agents include taxanes (e.g., paclitaxel and docetaxel) and vinca alkaloids (e.g., vinblastine, vincristine, vindesine, vinorelbine).

The skilled artisan will appreciate that the various chemotherapeutic agents described herein can be combined with any one or more of the activatable proproteins described herein, and used according to any one or more of the methods or compositions described herein.

Certain combination therapies employ at least one hormonal therapeutic agent. General examples of hormonal therapeutic agents include hormonal agonists and hormonal antagonists. Particular examples of hormonal agonists include progestogen (progestin), corticosteroids (e.g., prednisolone, methylprednisolone, dexamethasone), insulin like growth factors, VEGF derived angiogenic and lymphangiogenic factors (e.g., VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, androgens, estrogens, and somatostatin analogs. Examples of hormonal antagonists include hormone synthesis inhibitors such as aromatase inhibitors and gonadotropin-releasing hormone (GnRH)s agonists (e.g., leuprolide, goserelin, triptorelin, histrelin) including analogs thereof. Also included are hormone receptor antagonist such as selective estrogen receptor modulators (SERMs; e.g., tamoxifen, raloxifene, toremifene) and anti-androgens (e.g., flutamide, bicalutamide, nilutamide).

Also included are hormonal pathway inhibitors such as antibodies directed against hormonal receptors. Examples include inhibitors of the the IGF receptor (e.g., IGF-IR1) such as cixutumumab, dalotuzumab, figitumumab, ganitumab, istiratumab, and robatumumab; inhibitors of the vascular endothelial growth factor receptors 1, 2 or 3 (VEGFR1, VEGFR2 or VEGFR3) such as alacizumab pegol, bevacizumab, icrucumab, ramucirumab; inhibitors of the TGF-beta receptors R1, R2, and R3 such as fresolimumab and metelimumab; inhibitors of c-Met such as naxitamab; inhibitors of the EGF receptor such as cetuximab, depatuxizumab mafodotin, futuximab, imgatuzumab, laprituximab emtansine, matuzumab, modotuximab, necitumumab, nimotuzumab, panitumumab, tomuzotuximab, and zalutumumab; inhibitors of the FGF receptor such as aprutumab ixadotin and bemarituzumab; and inhibitors of the PDGF receptor such as olaratumab and tovetumab.

The skilled artisan will appreciate that the various hormonal therapeutic agents described herein can be combined with any one or more of the various activatable proproteins described herein, and used according to any one or more of the methods or compositions described herein.

Certain combination therapies employ at least one kinase inhibitor, including tyrosine kinase inhibitors. Examples of kinase inhibitors include, without limitation, adavosertib, afanitib, aflibercept, axitinib, bevacizumab, bosutinib, cabozantinib, cetuximab, cobimetinib, crizotinib, dasatinib, entrectinib, erdafitinib, erlotinib, fostamitinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, tofacitinib, trastuzumab, vandetanib, and vemuafenib.

The skilled artisan will appreciate that the various kinase inhibitors described herein can be combined with any one or more of the various activatable proproteins described herein, and used according to any one or more of the methods or compositions described herein.

In some embodiments, the methods and pharmaceutical compositions described herein increase median survival time of a subject by 4 weeks, 5 weeks, 6 weeks, 7 weeks, 8 weeks, 9 weeks, 10 weeks, 15 weeks, 20 weeks, 25 weeks, 30 weeks, 40 weeks, or longer. In certain embodiments, the methods and pharmaceutical compositions described herein increase median survival time of a subject by 1 year, 2 years, 3 years, or longer. In some embodiments, the methods and pharmaceutical compositions increase progression-free survival by 2 weeks, 3 weeks, 4 weeks, 5 weeks, 6 weeks, 7 weeks, 8 weeks, 9 weeks, 10 weeks or longer. In certain embodiments, the methods and pharmaceutical compositions described herein increase progression-free survival by 1 year, 2 years, 3 years, or longer.

In certain embodiments, the methods and therapeutic compositions described herein are sufficient to result in tumor regression, as indicated by a statistically significant decrease in the amount of viable tumor, for example, at least a 10%, 20%, 30%, 40%, 50% or greater decrease in tumor mass, or by altered (e.g., decreased with statistical significance) scan dimensions. In certain embodiments, the methods and therapeutic compositions described herein are sufficient to result in stable disease.

In some embodiments, the disease is a viral disease or viral infection. In certain embodiments, the viral infection is selected from one or more of human immunodeficiency virus (HIV), Hepatitis A, Hepatitis B, Hepatitis C, Hepatitis E, Caliciviruses associated diarrhoea, Rotavirus diarrhoea, Haemophilus influenzae B pneumonia and invasive disease, influenza, measles, mumps, rubella, Parainfluenza associated pneumonia, Respiratory syncytial virus (RSV) pneumonia, Severe Acute Respiratory Syndrome (SARS), Human papillomavirus, Herpes simplex type 2 genital ulcers, Dengue Fever, Japanese encephalitis, Tick-borne encephalitis, West-Nile virus associated disease, Yellow Fever, Epstein-Barr virus, Lassa fever, Crimean-Congo haemorrhagic fever, Ebola haemorrhagic fever, Marburg haemorrhagic fever, Rabies, Rift Valley fever, Smallpox, upper and lower respiratory infections, and poliomyelitis. In specific embodiments, the subject is HIV-positive. In some embodiments, the methods and pharmaceutical compositions described herein increase an anti-viral immune response by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control.

In some embodiments, the immune disorder is selected from one or more of type 1 diabetes, vasculitis, and an immunodeficiency. In some embodiments, the methods and pharmaceutical compositions described herein improve immune function in the subject, for example, by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control.

In certain embodiments, the methods and therapeutic compositions described herein are sufficient to result in clinically relevant reduction in symptoms of a particular disease indication known to the skilled clinician.

For in vivo use, as noted above, for the treatment of human or non-human mammalian disease or testing, the agents described herein are generally incorporated into one or more therapeutic or pharmaceutical compositions prior to administration, including veterinary therapeutic compositions.

Thus, certain embodiments relate to pharmaceutical or therapeutic compositions that comprise at least one activatable proprotein, as described herein. In some instances, a pharmaceutical or therapeutic composition comprises one or more of the activatable proproteins described herein in combination with a pharmaceutically- or physiologically-acceptable carrier or excipient. Certain pharmaceutical or therapeutic compositions further comprise at least one additional agent, for example, a chemotherapeutic agent, a hormonal therapeutic agent, and/or a kinase inhibitor as described herein.

Some therapeutic compositions comprise (and certain methods utilize) only one activatable proprotein. Certain therapeutic compositions comprise (and certain methods utilize) a mixture of at least two, three, four, or five different activatable proproteins.

In particular embodiments, the pharmaceutical or therapeutic compositions comprising at least one activatable proprotein is substantially pure on a protein basis or a weight-weight basis, for example, the composition has a purity of at least about 80%, 85%, 90%, 95%, 98%, or 99% on a protein basis or a weight-weight basis.

In some embodiments, the activatable proproteins described herein do not form aggregates, have a desired solubility, and/or have an immunogenicity profile that is suitable for use in humans, as known in the art. Thus, in some embodiments, the therapeutic composition comprising an activatable proprotein is substantially aggregate-free. For example, certain compositions comprise less than about 10% (on a protein basis) high molecular weight aggregated proteins, or less than about 5% high molecular weight aggregated proteins, or less than about 4% high molecular weight aggregated proteins, or less than about 3% high molecular weight aggregated proteins, or less than about 2% high molecular weight aggregated proteins, or less than about 1% high molecular weight aggregated proteins. Some compositions comprise an activatable proprotein that is at least about 50%, about 60%, about 70%, about 80%, about 90% or about 95% monodisperse with respect to its apparent molecular mass.

In some embodiments, the activatable proprotein are concentrated to about or at least about 0.1 mg/ml, 0.2 mg/ml, 0.3 mg/ml, 0.4 mg/ml, 0.5 mg/ml, 0.6, 0.7, 0.8, 0.9, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11, 12, 13, 14 or 15 mg/ml and are formulated for biotherapeutic uses.

To prepare a therapeutic or pharmaceutical composition, an effective or desired amount of one or more agents is mixed with any pharmaceutical carrier(s) or excipient known to those skilled in the art to be suitable for the particular agent and/or mode of administration. A pharmaceutical carrier may be liquid, semi-liquid or solid. Solutions or suspensions used for parenteral, intradermal, subcutaneous or topical application may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously (e.g., by IV infusion), suitable carriers include physiological saline or phosphate buffered saline (PBS), and solutions containing thickening and solubilizing agents, such as glucose, polyethylene glycol, polypropylene glycol and mixtures thereof.

Administration of agents described herein, in pure form or in an appropriate therapeutic or pharmaceutical composition, can be carried out via any of the accepted modes of administration of agents for serving similar utilities. The therapeutic or pharmaceutical compositions can be prepared by combining an agent-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other small molecules as described elsewhere herein) and/or suitable excipients such as salts, buffers and stabilizers may, but need not, be present within the composition.

Administration may be achieved by a variety of different routes, including oral, parenteral, nasal, intravenous, intradermal, intramuscular, subcutaneous or topical. Preferred modes of administration depend upon the nature of the condition to be treated or prevented. Particular embodiments include administration by IV infusion.

Carriers can include, for example, pharmaceutically- or physiologically-acceptable carriers, excipients, or stabilizers that are non-toxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In some embodiments, one or more agents can be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). The particle(s) or liposomes may further comprise other therapeutic or diagnostic agents.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Typical routes of administering these and related therapeutic or pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Therapeutic or pharmaceutical compositions according to certain embodiments of the present disclosure are formulated so as to allow the active ingredients contained therein to be bioavailable upon administration of the composition to a subject or patient. Compositions that will be administered to a subject or patient may take the form of one or more dosage units, where for example, a tablet may be a single dosage unit, and a container of a herein described agent in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will typically contain a therapeutically effective amount of an agent described herein, for treatment of a disease or condition of interest.

A therapeutic or pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid. Certain embodiments include sterile, injectable solutions.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The therapeutic or pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid therapeutic or pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid therapeutic or pharmaceutical composition intended for either parenteral or oral administration should contain an amount of an agent such that a suitable dosage will be obtained. Typically, this amount is at least 0.01% of the agent of interest in the composition. When intended for oral administration, this amount may be varied to be between 0.1 and about 70% of the weight of the composition. Certain oral therapeutic or pharmaceutical compositions contain between about 4% and about 75% of the agent of interest. In certain embodiments, therapeutic or pharmaceutical compositions and preparations are prepared so that a parenteral dosage unit contains between 0.01 to 10% by weight of the agent of interest prior to dilution.

The therapeutic or pharmaceutical compositions may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a therapeutic or pharmaceutical composition for topical administration. If intended for transdermal administration, the composition may include a transdermal patch or iontophoresis device.

The therapeutic or pharmaceutical compositions may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter, and polyethylene glycol.

The therapeutic or pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The therapeutic or pharmaceutical compositions in solid or liquid form may include a component that binds to agent and thereby assists in the delivery of the compound. Suitable components that may act in this capacity include monoclonal or polyclonal antibodies, one or more proteins or a liposome.

The therapeutic or pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The compositions described herein may be prepared with carriers that protect the agents against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The therapeutic or pharmaceutical compositions may be prepared by methodology well known in the pharmaceutical art. For example, a therapeutic or pharmaceutical composition intended to be administered by injection may comprise one or more of salts, buffers and/or stabilizers, with sterile, distilled water so as to form a solution. A surfactant may be added to facilitate the formation of a homogeneous solution or suspension. Surfactants are compounds that non-covalently interact with the agent so as to facilitate dissolution or homogeneous suspension of the agent in the aqueous delivery system.

The therapeutic or pharmaceutical or IVIG compositions may be administered in a therapeutically effective amount, which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy. In some instances, a therapeutically effective daily dose is (for a 70 kg mammal) from about 0.001 mg/kg (i.e., ˜0.07 mg) to about 100 mg/kg (i.e., ˜7.0 g); preferably a therapeutically effective dose is (for a 70 kg mammal) from about 0.01 mg/kg (i.e., ˜0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., ˜1.75 g). In some embodiments, the therapeutically effective dose is administered on a weekly, bi-weekly, or monthly basis. In specific embodiments, the therapeutically effective dose is administered on a weekly, bi-weekly, or monthly basis, for example, at a dose of about 1-10 or 1-5 mg/kg, or about 1, 2, 3, 4, 5, 6, 7, 8, 9, or 10 mg/kg.

The combination therapies described herein may include administration of a single pharmaceutical dosage formulation, which contains an activatable proprotein and an additional therapeutic agent (e.g., chemotherapeutic agent, hormonal therapeutic agent, kinase inhibitor), as well as administration of compositions comprising an activatable proprotein and an additional therapeutic agent in its own separate pharmaceutical dosage formulation. For example, an activatable proprotein and additional therapeutic agent can be administered to the subject together in a single oral dosage composition such as a tablet or capsule, or each agent administered in separate oral dosage formulations. Similarly, an activatable proprotein and additional therapeutic agent can be administered to the subject together in a single parenteral dosage composition such as in a saline solution or other physiologically acceptable solution, or each agent administered in separate parenteral dosage formulations. As another example, for cell-based therapies, an activatable proprotein can be mixed with the cells prior to administration, administered as part of a separate composition, or both. Where separate dosage formulations are used, the compositions can be administered at essentially the same time, i.e., concurrently, or at separately staggered times, i.e., sequentially and in any order; combination therapy is understood to include all these regimens.

Also included are patient care kits, comprising (a) at least one activatable proprotein, as described herein; and optionally (b) at least one additional therapeutic agent (e.g., chemotherapeutic agent, hormonal therapeutic agent, kinase inhibitor). In certain kits, (a) and (b) are in separate therapeutic compositions. In some kits, (a) and (b) are in the same therapeutic composition.

The kits herein may also include a one or more additional therapeutic agents or other components suitable or desired for the indication being treated, or for the desired diagnostic application. The kits herein can also include one or more syringes or other components necessary or desired to facilitate an intended mode of delivery (e.g., stents, implantable depots, etc.).

In some embodiments, a patient care kit contains separate containers, dividers, or compartments for the composition(s) and informational material(s). For example, the composition(s) can be contained in a bottle, vial, or syringe, and the informational material(s) can be contained in association with the container. In some embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of an activatable proprotein and optionally at least one additional therapeutic agent. For example, the kit includes a plurality of syringes, ampules, foil packets, or blister packs, each containing a single unit dose of an activatable proprotein and optionally at least one additional therapeutic agent. The containers of the kits can be air tight, waterproof (e.g., impermeable to changes in moisture or evaporation), and/or light-tight.

The patient care kit optionally includes a device suitable for administration of the composition, e.g., a syringe, inhalant, dropper (e.g., eye dropper), swab (e.g., a cotton swab or wooden swab), or any such delivery device. In some embodiments, the device is an implantable device that dispenses metered doses of the agent(s). Also included are methods of providing a kit, e.g., by combining the components described herein.

Expression and Purification Systems

Certain embodiments include methods and related compositions for expressing and purifying an activatable proprotein homodimer described herein. Such recombinant activatable proproteins can be conveniently prepared using standard protocols as described for example in Sambrook, et al., (1989, supra), in particular Sections 16 and 17; Ausubel et al., (1994, supra), in particular Chapters 10 and 16; and Coligan et al., Current Protocols in Protein Science (John Wiley & Sons, Inc. 1995-1997), in particular Chapters 1, 5 and 6. As one general example, activatable proproteins may be prepared by a procedure including one or more of the steps of: (a) preparing one or more vectors or constructs comprising one or more polynucleotide sequences that encode a first and second polypeptide described herein (see, e.g., Table S15), which is/are operably linked to one or more regulatory elements; (b) introducing the one or more vectors or constructs into one or more host cells; (c) culturing the one or more host cell to express the first and second polypeptides, which bind together to form an activatable proprotein; and (d) isolating the activatable proprotein from the host cell.

To express a desired polypeptide, a nucleotide sequence encoding a first and/or second polypeptide chain of an activatable proprotein may be inserted into appropriate expression vector(s), i.e., vector(s) which contain the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems are known and may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems, including mammalian cell and more specifically human cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, vectors which direct high level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of β-galactosidase so that a hybrid protein is produced; pIN vectors (Van Heeke & Schuster, J. Biol. Chem. 264:5503 5509 (1989)); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

Certain embodiments employ E. coli-based expression systems (see, e.g., Structural Genomics Consortium et al., Nature Methods. 5:135-146, 2008). These and related embodiments may rely partially or totally on ligation-independent cloning (LIC) to produce a suitable expression vector. In specific embodiments, protein expression may be controlled by a T7 RNA polymerase (e.g., pET vector series). These and related embodiments may utilize the expression host strain BL21(DE3), a XDE3 lysogen of BL21 that supports T7-mediated expression and is deficient in ion and ompT proteases for improved target protein stability. Also included are expression host strains carrying plasmids encoding tRNAs rarely used in E. coli, such as ROSETTA™ (DE3) and Rosetta 2 (DE3) strains. Cell lysis and sample handling may also be improved using reagents sold under the trademarks BENZONASE® nuclease and BUGBUSTER® Protein Extraction Reagent. For cell culture, auto-inducing media can improve the efficiency of many expression systems, including high-throughput expression systems. Media of this type (e.g., OVERNIGHT EXPRESS™ Autoinduction System) gradually elicit protein expression through metabolic shift without the addition of artificial inducing agents such as IPTG. Particular embodiments employ hexahistidine tags (such as those sold under the trademark HIS•TAG® fusions), followed by immobilized metal affinity chromatography (IMAC) purification, or related techniques. In certain aspects, however, clinical grade proteins can be isolated from E. coli inclusion bodies, without or without the use of affinity tags (see, e.g., Shimp et al., Protein Expr Purif. 50:58-67, 2006). As a further example, certain embodiments may employ a cold-shock induced E. coli high-yield production system, because over-expression of proteins in Escherichia coli at low temperature improves their solubility and stability (see, e.g., Qing et al., Nature Biotechnology. 22:877-882, 2004).

Also included are high-density bacterial fermentation systems. For example, high cell density cultivation of Ralstonia eutropha allows protein production at cell densities of over 150 g/L, and the expression of recombinant proteins at titers exceeding 10 g/L.

In the yeast Saccharomyces cerevisiae, a number of vectors containing constitutive or inducible promoters such as alpha factor, alcohol oxidase, and PGH may be used. For reviews, see Ausubel et al. (supra) and Grant et al., Methods Enzymol. 153:516-544 (1987). Also included are Pichia pandoris expression systems (see, e.g., Li et al., Nature Biotechnology. 24, 210-215, 2006; and Hamilton et al., Science, 301:1244, 2003). Certain embodiments include yeast systems that are engineered to selectively glycosylate proteins, including yeast that have humanized N-glycosylation pathways, among others (see, e.g., Hamilton et al., Science. 313:1441-1443, 2006; Wildt et al., Nature Reviews Microbiol. 3:119-28, 2005; and Gerngross et al., Nature-Biotechnology. 22:1409-1414, 2004; U.S. Pat. Nos. 7,629,163; 7,326,681; and 7,029,872). Merely by way of example, recombinant yeast cultures can be grown in Fernbach Flasks or 15 L, 50 L, 100 L, and 200 L fermentors, among others.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, EMBO J. 6:307-311 (1987)). Alternatively, plant promoters such as the small subunit of RUBISCO or heat shock promoters may be used (Coruzzi et al., EMBO J. 3:1671-1680 (1984); Broglie et al., Science 224:838-843 (1984); and Winter et al., Results Probl. Cell Differ. 17:85-105 (1991)). These constructs can be introduced into plant cells by direct DNA transformation or pathogen-mediated transfection. Such techniques are described in a number of generally available reviews (see, e.g., Hobbs in McGraw Hill, Yearbook of Science and Technology, pp. 191-196 (1992)).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia cells. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia cells in which the polypeptide of interest may be expressed (Engelhard et al., Proc. Natl. Acad. Sci. U.S.A. 91:3224-3227 (1994)). Also included are baculovirus expression systems, including those that utilize SF9, SF21, and T. ni cells (see, e.g., Murphy and Piwnica—Worms, Curr Protoc Protein Sci. Chapter 5:Unit5.4, 2001). Insect systems can provide post-translation modifications that are similar to mammalian systems.

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan & Shenk, Proc. Natl. Acad. Sci. U.S.A. 81:3655-3659 (1984)). In addition, transcription enhancers, such as the Rous sarcoma virus (RSV) enhancer, may be used to increase expression in mammalian host cells.

Examples of useful mammalian host cell lines include monkey kidney CV1 line transformed by SV40 (COS-7, ATCC CRL 1651); human embryonic kidney line (293 or 293 cells sub-cloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR-CHO cells (Urlaub et al., PNAS USA 77:4216 (1980)); and myeloma cell lines such as NSO and Sp2/0. For a review of certain mammalian host cell lines suitable for protein production, see, e.g., Yazaki and Wu, Methods in Molecular Biology, Vol. 248 (B. K. C Lo, ed., Humana Press, Totowa, N.J., 2003), pp. 255-268. Certain preferred mammalian cell expression systems include CHO and HEK293-cell based expression systems. Mammalian expression systems can utilize attached cell lines, for example, in T-flasks, roller bottles, or cell factories, or suspension cultures, for example, in 1 L and 5 L spinners, 5 L, 14 L, 40 L, 100 L and 200 L stir tank bioreactors, or 20/50 L and 100/200 L WAVE bioreactors, among others known in the art.

Also included is the cell-free expression of proteins. These and related embodiments typically utilize purified RNA polymerase, ribosomes, tRNA and ribonucleotides; these reagents may be produced by extraction from cells or from a cell-based expression system.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf. et al., Results Probl. Cell Differ. 20:125-162 (1994)).

In addition, a host cell strain may be chosen for its ability to modulate the expression of the inserted sequences or to process the expressed protein in the desired fashion. Such modifications of the polypeptide include, but are not limited to, post-translational modifications such as acetylation, carboxylation, glycosylation, phosphorylation, lipidation, and acylation. Post-translational processing which cleaves a “prepro” form of the protein may also be used to facilitate correct insertion, folding and/or function. Different host cells such as yeast, CHO, HeLa, MDCK, HEK293, and W138, in addition to bacterial cells, which have or even lack specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for about 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type. Transient production, such as by transient transfection or infection, can also be employed. Exemplary mammalian expression systems that are suitable for transient production include HEK293 and CHO-based systems.

Any number of selection systems may be used to recover transformed or transduced cell lines. These include, but are not limited to, the herpes simplex virus thymidine kinase (Wigler et al., Cell 11:223-232 (1977)) and adenine phosphoribosyltransferase (Lowy et al., Cell 22:817-823 (1990)) genes which can be employed in tk− or aprt− cells, respectively. Also, antimetabolite, antibiotic or herbicide resistance can be used as the basis for selection; for example, dhfr which confers resistance to methotrexate (Wigler et al., Proc. Natl. Acad. Sci. U.S.A. 77:3567-70 (1980)); npt, which confers resistance to the aminoglycosides, neomycin and G-418 (Colbere-Garapin et al., J. Mol. Biol. 150:1-14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. U.S.A. 85:8047-51 (1988)). The use of visible markers has gained popularity with such markers as green fluorescent protein (GFP) and other fluorescent proteins (e.g., RFP, YFP), anthocyanins, β-glucuronidase and its substrate GUS, and luciferase and its substrate luciferin, being widely used not only to identify transformants, but also to quantify the amount of transient or stable protein expression attributable to a specific vector system (see, e.g., Rhodes et al., Methods Mol. Biol. 55:121-131 (1995)).

Also included are high-throughput protein production systems, or micro-production systems. Certain aspects may utilize, for example, hexa-histidine fusion tags for protein expression and purification on metal chelate-modified slide surfaces or MagneHis Ni-Particles (see, e.g., Kwon et al., BMC Biotechnol. 9:72, 2009; and Lin et al., Methods Mol Biol. 498:129-41, 2009)). Also included are high-throughput cell-free protein expression systems (see, e.g., Sitaraman et al., Methods Mol Biol. 498:229-44, 2009).

A variety of protocols for detecting and measuring the expression of polynucleotide-encoded products, using binding agents or antibodies such as polyclonal or monoclonal antibodies specific for the product, are known in the art. Examples include enzyme-linked immunosorbent assay (ELISA), western immunoblots, radioimmunoassays (RIA), and fluorescence activated cell sorting (FACS). These and other assays are described, among other places, in Hampton et al., Serological Methods, a Laboratory Manual (1990) and Maddox et al., J. Exp. Med. 158:1211-1216 (1983).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with one or more polynucleotide sequences of interest may be cultured under conditions suitable for the expression and recovery of the protein from cell culture. Certain specific embodiments utilize serum free cell expression systems. Examples include HEK293 cells and CHO cells that can grown on serum free medium (see, e.g., Rosser et al., Protein Expr. Purif. 40:237-43, 2005; and U.S. Pat. No. 6,210,922).

An activatable proprotein produced by a recombinant cell may be secreted or contained intracellularly depending on the sequence and/or the vector used. As will be understood by those of skill in the art, expression vectors containing polynucleotides may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification and/or detection of soluble proteins. Examples of such domains include cleavable and non-cleavable affinity purification and epitope tags such as avidin, FLAG tags, poly-histidine tags (e.g., 6×His), cMyc tags, V5-tags, glutathione S-transferase (GST) tags, and others.

The protein produced by a recombinant cell can be purified and characterized according to a variety of techniques known in the art. Exemplary systems for performing protein purification and analyzing protein purity include fast protein liquid chromatography (FPLC) (e.g., AKTA and Bio-Rad FPLC systems), high-pressure liquid chromatography (HPLC) (e.g., Beckman and Waters HPLC). Exemplary chemistries for purification include ion exchange chromatography (e.g., Q, S), size exclusion chromatography, salt gradients, affinity purification (e.g., Ni, Co, FLAG, maltose, glutathione, protein A/G), gel filtration, reverse-phase, ceramic HYPERD® ion exchange chromatography, and hydrophobic interaction columns (HIC), among others known in the art. Also included are analytical methods such as SDS-PAGE (e.g., coomassie, silver stain), immunoblot, Bradford, and ELISA, which may be utilized during any step of the production or purification process, typically to measure the purity of the protein composition.

Also included are methods of concentrating activatable proproteins, and composition comprising concentrated soluble activatable proproteina. In some aspects, such concentrated solutions of at least tone activatable proprotein comprise proteins at a concentration of about or at least about 5 mg/mL, 8 mg/mL, 10 mg/mL, 15 mg/mL. 20 mg/mL, or more.

In some aspects, such compositions may be substantially monodisperse, meaning that an activatable proprotein exists primarily (i.e., at least about 90%, or greater) in one apparent molecular weight form when assessed for example, by size exclusion chromatography, dynamic light scattering, or analytical ultracentrifugation.

In some aspects, such compositions have a purity (on a protein basis) of at least about 90%, or in some aspects at least about 95% purity, or in some embodiments, at least 98% purity. Purity may be determined via any routine analytical method as known in the art.

In some aspects, such compositions have a high molecular weight aggregate content of less than about 10%, compared to the total amount of protein present, or in some embodiments such compositions have a high molecular weight aggregate content of less than about 5%, or in some aspects such compositions have a high molecular weight aggregate content of less than about 3%, or in some embodiments a high molecular weight aggregate content of less than about 1%. High molecular weight aggregate content may be determined via a variety of analytical techniques including for example, by size exclusion chromatography, dynamic light scattering, or analytical ultracentrifugation.

Examples of concentration approaches contemplated herein include lyophilization, which is typically employed when the solution contains few soluble components other than the protein of interest. Lyophilization is often performed after HPLC run, and can remove most or all volatile components from the mixture. Also included are ultrafiltration techniques, which typically employ one or more selective permeable membranes to concentrate a protein solution. The membrane allows water and small molecules to pass through and retains the protein; the solution can be forced against the membrane by mechanical pump, gas pressure, or centrifugation, among other techniques.

In certain embodiments, an activatable proprotein in a composition has a purity of at least about 90%, as measured according to routine techniques in the art. In certain embodiments, such as diagnostic compositions or certain pharmaceutical or therapeutic compositions, an activatable proprotein composition has a purity of at least about 95%, or at least about 97% or 98% or 99%. In some embodiments, such as when being used as reference or research reagents, activatable proproteins can be of lesser purity, and may have a purity of at least about 50%, 60%, 70%, or 80%. Purity can be measured overall or in relation to selected components, such as other proteins, e.g., purity on a protein basis.

Purified activatable proproteins can also be characterized according to their biological characteristics. Binding affinity and binding kinetics can be measured according to a variety of techniques known in the art, such as Biacore® and related technologies that utilize surface plasmon resonance (SPR), an optical phenomenon that enables detection of unlabeled interactants in real time. SPR-based biosensors can be used in determination of active concentration, screening and characterization in terms of both affinity and kinetics. The presence or levels of one or more biological activities can be measured according to cell-based assays, including those that utilize at least one IL-2 receptor and/or IL-15 receptor, which is optionally functionally coupled to a readout or indicator, such as a fluorescent or luminescent indicator of biological activity, as described herein.

In certain embodiments, as noted above, an activatable proprotein composition is substantially endotoxin free, including, for example, about 95% endotoxin free, preferably about 99% endotoxin free, and more preferably about 99.99% endotoxin free. The presence of endotoxins can be detected according to routine techniques in the art, as described herein. In specific embodiments, an activatable proprotein composition is made from a eukaryotic cell such as a mammalian or human cell in substantially serum free media. In certain embodiments, as noted herein, an activatable proprotein composition has an endotoxin content of less than about 10 EU/mg of activatable proprotein, or less than about 5 EU/mg of activatable proprotein, less than about 3 EU/mg of activatable proprotein, or less than about 1 EU/mg of activatable proprotein.

In certain embodiments, an activatable proprotein composition comprises less than about 10% wt/wt high molecular weight aggregates, or less than about 5% wt/wt high molecular weight aggregates, or less than about 2% wt/wt high molecular weight aggregates, or less than about or less than about 1% wt/wt high molecular weight aggregates.

Also included are protein-based analytical assays and methods, which can be used to assess, for example, protein purity, size, solubility, and degree of aggregation, among other characteristics. Protein purity can be assessed a number of ways. For instance, purity can be assessed based on primary structure, higher order structure, size, charge, hydrophobicity, and glycosylation. Examples of methods for assessing primary structure include N- and C-terminal sequencing and peptide-mapping (see, e.g., Allen et al., Biologicals. 24:255-275, 1996)). Examples of methods for assessing higher order structure include circular dichroism (see, e.g., Kelly et al., Biochim Biophys Acta. 1751:119-139, 2005), fluorescent spectroscopy (see, e.g., Meagher et al., J. Biol. Chem. 273:23283-89, 1998), FT-IR, amide hydrogen-deuterium exchange kinetics, differential scanning calorimetry, NMR spectroscopy, immunoreactivity with conformationally sensitive antibodies. Higher order structure can also be assessed as a function of a variety of parameters such as pH, temperature, or added salts. Examples of methods for assessing protein characteristics such as size include analytical ultracentrifugation and size exclusion HPLC (SEC-HPLC), and exemplary methods for measuring charge include ion-exchange chromatography and isolectric focusing. Hydrophobicity can be assessed, for example, by reverse-phase HPLC and hydrophobic interaction chromatography HPLC. Glycosylation can affect pharmacokinetics (e.g., clearance), conformation or stability, receptor binding, and protein function, and can be assessed, for example, by mass spectrometry and nuclear magnetic resonance (NMR) spectroscopy.

As noted above, certain embodiments include the use of SEC-HPLC to assess protein characteristics such as purity, size (e.g., size homogeneity) or degree of aggregation, and/or to purify proteins, among other uses. SEC, also including gel-filtration chromatography (GFC) and gel-permeation chromatography (GPC), refers to a chromatographic method in which molecules in solution are separated in a porous material based on their size, or more specifically their hydrodynamic volume, diffusion coefficient, and/or surface properties. The process is generally used to separate biological molecules, and to determine molecular weights and molecular weight distributions of polymers. Typically, a biological or protein sample (such as a protein extract produced according to the protein expression methods provided herein and known in the art) is loaded into a selected size-exclusion column with a defined stationary phase (the porous material), preferably a phase that does not interact with the proteins in the sample. In certain aspects, the stationary phase is composed of inert particles packed into a dense three-dimensional matrix within a glass or steel column. The mobile phase can be pure water, an aqueous buffer, an organic solvent, or a mixture thereof. The stationary-phase particles typically have small pores and/or channels which only allow molecules below a certain size to enter. Large particles are therefore excluded from these pores and channels, and their limited interaction with the stationary phase leads them to elute as a “totally-excluded” peak at the beginning of the experiment. Smaller molecules, which can fit into the pores, are removed from the flowing mobile phase, and the time they spend immobilized in the stationary-phase pores depends, in part, on how far into the pores they penetrate. Their removal from the mobile phase flow causes them to take longer to elute from the column and results in a separation between the particles based on differences in their size. A given size exclusion column has a range of molecular weights that can be separated. Overall, molecules larger than the upper limit will not be trapped by the stationary phase, molecules smaller than the lower limit will completely enter the solid phase and elute as a single band, and molecules within the range will elute at different rates, defined by their properties such as hydrodynamic volume. For examples of these methods in practice with pharmaceutical proteins, see Bruner et al., Journal of Pharmaceutical and Biomedical Analysis. 15: 1929-1935, 1997.

Protein purity for clinical applications is also discussed, for example, by Anicetti et al. (Trends in Biotechnology. 7:342-349, 1989). More recent techniques for analyzing protein purity include, without limitation, the LabChip GXII, an automated platform for rapid analysis of proteins and nucleic acids, which provides high throughput analysis of titer, sizing, and purity analysis of proteins. In certain non-limiting embodiments, clinical grade activatable proproteins can be obtained by utilizing a combination of chromatographic materials in at least two orthogonal steps, among other methods (see, e.g., Therapeutic Proteins: Methods and Protocols. Vol. 308, Eds., Smales and James, Humana Press Inc., 2005). Typically, protein agents (e.g., activatable proprotein) are substantially endotoxin-free, as measured according to techniques known in the art and described herein.

Protein solubility assays are also included. Such assays can be utilized, for example, to determine optimal growth and purification conditions for recombinant production, to optimize the choice of buffer(s), and to optimize the choice of activatable proproteins and variants thereof. Solubility or aggregation can be evaluated according to a variety of parameters, including temperature, pH, salts, and the presence or absence of other additives. Examples of solubility screening assays include, without limitation, microplate-based methods of measuring protein solubility using turbidity or other measure as an end point, high-throughput assays for analysis of the solubility of purified recombinant proteins (see, e.g., Stenvall et al., Biochim Biophys Acta. 1752:6-10, 2005), assays that use structural complementation of a genetic marker protein to monitor and measure protein folding and solubility in vivo (see, e.g., Wigley et al., Nature Biotechnology. 19:131-136, 2001), and electrochemical screening of recombinant protein solubility in Escherichia coli using scanning electrochemical microscopy (SECM) (see, e.g., Nagamine et al., Biotechnology and Bioengineering. 96:1008-1013, 2006), among others. Activatable proprotein with increased solubility (or reduced aggregation) can be identified or selected for according to routine techniques in the art, including simple in vivo assays for protein solubility (see, e.g., Maxwell et al., Protein Sci. 8:1908-11, 1999).

Protein solubility and aggregation can also be measured by dynamic light scattering techniques. Aggregation is a general term that encompasses several types of interactions or characteristics, including soluble/insoluble, covalent/noncovalent, reversible/irreversible, and native/denatured interactions and characteristics. For protein therapeutics, the presence of aggregates is typically considered undesirable because of the concern that aggregates may cause an immunogenic reaction (e.g., small aggregates), or may cause adverse events on administration (e.g., particulates). Dynamic light scattering refers to a technique that can be used to determine the size distribution profile of small particles in suspension or polymers such as proteins in solution. This technique, also referred to as photon correlation spectroscopy (PCS) or quasi-elastic light scattering (QELS), uses scattered light to measure the rate of diffusion of the protein particles. Fluctuations of the scattering intensity can be observed due to the Brownian motion of the molecules and particles in solution. This motion data can be conventionally processed to derive a size distribution for the sample, wherein the size is given by the Stokes radius or hydrodynamic radius of the protein particle. The hydrodynamic size depends on both mass and shape (conformation). Dynamic scattering can detect the presence of very small amounts of aggregated protein (<0.01% by weight), even in samples that contain a large range of masses. It can also be used to compare the stability of different formulations, including, for example, applications that rely on real-time monitoring of changes at elevated temperatures. Accordingly, certain embodiments include the use of dynamic light scattering to analyze the solubility and/or presence of aggregates in a sample that contains an activatable proprotein of the present disclosure.

1. An activatable proprotein homodimer, comprising a first polypeptide and a second polypeptide, wherein the first polypeptide and the second polypeptide comprise, in a C- to N-terminal orientation, a binding moiety, a first linker, a cytokine, a second linker, and a cytokine receptor,
wherein the binding moiety of the first polypeptide binds to the binding moiety of the second polypeptide, wherein the cytokine of the first polypeptide binds to the cytokine receptor of the second polypeptide, and wherein the cytokine receptor of the first polypeptide binds to the cytokine of the second polypeptide, wherein said binding masks a binding site of the cytokine that otherwise binds to its wild-type cognate receptor on the surface of an immune cell in vitro or in vivo, and wherein at least one of the first or the second linker is a cleavable linker.

2. The activatable proprotein homodimer of claim 1, wherein the cytokine receptor is a variant that comprises one or more amino acid alterations relative to the corresponding wild-type cytokine receptor, and which has reduced binding affinity to the cytokine relative to that of the wild-type cytokine receptor.

3. The activatable proprotein homodimer of claim 2, wherein the cytokine receptor variant has reduced binding affinity to the cytokine of about or at least about 2-fold, 5-fold, 10-fold, 50-fold, 100-fold, 1000-fold or more, relative to the binding affinity of the wild-type cytokine receptor to the cytokine.

4. The activatable proprotein of any one of claims 1-3, wherein the cytokine is a variant that comprises one or more amino acid alterations relative to the corresponding wild-type cytokine, and has altered (increased, decreased) binding affinity to its wild-type cognate receptor on the surface of the immune cell in vitro or in vivo.

5. The activatable proprotein homodimer of claim 4, wherein the cytokine variant has altered (increased, decreased) binding affinity to its wild-type cognate receptor of about or at least about 2-fold, 5-fold, 10-fold, 50-fold, 100-fold, 1000-fold or more, relative to the binding affinity of the wild-type cytokine to the wild-type cognate receptor.

6. The activatable proprotein homodimer of any one of claims 1-5, wherein the cytokine receptor comprises, consists, or consists essentially of the extracellular domain (ECD) of the cytokine receptor.

7. The activatable proprotein homodimer of any one of claims 1-6, wherein:
the cytokine comprises an interleukin-2 (IL-2) protein, the cytokine receptor comprises an IL-2Rβ protein, and the wild-type cognate receptor comprises IL-2Rβ/λc on the surface of the immune cell;
the cytokine comprises an interleukin-7 (IL-7) protein, the cytokine receptor comprises an IL-7Rα protein, and the wild-type cognate receptor comprises IL-7Rα/λc on the surface of the immune cell;
the cytokine comprises an interleukin-15 (IL-15) protein, the cytokine receptor comprises an IL-15Rβ protein, and the wild-type cognate receptor comprises IL-15Rβ/γc on the surface of the immune cell;
the cytokine comprises an interleukin-21 (IL-21) protein, the cytokine receptor comprises an IL-21R protein, and the wild-type cognate receptor comprises IL-21R/λc on the surface of the immune cell;
the cytokine comprises a type I interferon (IFN) protein, the cytokine receptor comprises a IFNAR2 protein, and the wild-type cognate receptor comprises IFNAR1/IFNAR2 on the surface of the immune cell;
the cytokine comprises a type II IFN protein, the cytokine receptor comprises a IFNGR1 protein, and the wild-type cognate receptor comprises IFNGR1/IFNGR2 on the surface of the immune cell; or
the cytokine comprises a type III IFN protein, the cytokine receptor comprises a IL10Rβ or IFN-λR1 protein, and the wild-type cognate receptor comprises IL10Rβ/IFN-λR1 on the surface of the immune cell.

8. The activatable proprotein homodimer of claim 7, wherein
the IL-2 protein or variant thereof comprises, consists, or consists essentially of an amino acid sequence that is at least 80, 85, 90, 95, 98, or 100% identical to a sequence selected from Table S1; and
the IL-2Rβ protein or variant thereof comprises, consists, or consists essentially of an amino acid sequence that is at least 80, 85, 90, 95, 98, or 100% identical to a sequence selected from Table S8.

9. The activatable proprotein homodimer of claim 7, wherein
the IL-7 protein or variant thereof comprises, consists, or consists essentially of an amino acid sequence that is at least 80, 85, 90, 95, 98, or 100% identical to a sequence selected from Table S2, and optionally comprises or retains a substitution at E106, as defined by the mature IL-7 sequence, including an E106A substitution; and
the IL-7Rα protein or variant thereof comprises, consists, or consists essentially of an amino acid sequence that is at least 80, 85, 90, 95, 98, or 100% identical to a sequence selected from Table S9, which optionally comprises or retains a substitution at any one or more of 146, K77, and/or L80, as defined by the mature IL-7Rα sequence, optionally an I46T substitution, a K77A substitution, and/or an L80A substitution.

10. The activatable proprotein homodimer of claim 7, wherein
the IL-15 protein or variant thereof comprises, consists, or consists essentially of an amino acid sequence that is at least 80, 85, 90, 95, 98, or 100% identical to a sequence selected from Table S3; and
the IL-15Rβ protein or variant thereof comprises, consists, or consists essentially of an amino acid sequence that is at least 80, 85, 90, 95, 98, or 100% identical to a sequence selected from Table S10.

11. The activatable proprotein homodimer of claim 7, wherein
the IL-21 protein or variant thereof comprises, consists, or consists essentially of an amino acid sequence that is at least 80, 85, 90, 95, 98, or 100% identical to a sequence selected from Table S4; and
the IL-21R protein or variant thereof comprises, consists, or consists essentially of an amino acid sequence that is at least 80, 85, 90, 95, 98, or 100% identical to a sequence selected from Table S11, which optionally comprises or retains a substitution at any one or more of Y36, M70, D72, D73, and/or Y129, as defined by the mature IL-21R sequence, optionally any one or more of Y36A, M70A, D72A, D73A, and/or Y129A.

12. The activatable proprotein homodimer of claim 7, wherein the type I IFN protein is selected from IFNA1, IFNA2, IFNA4, IFNA5, IFNA6, IFNA7, IFNA8, IFNA10, IFNA13, IFNA14, IFNA16, IFNA17, IFNA21, IFNB1, IFNB3, IFNW1, and IFNK, including cytokine variants thereof, optionally wherein the IFNA1, IFNA2, IFNA4, IFNA5, IFNA6, IFNA7, IFNA8, IFNA10, IFNA13, IFNA14, IFNA16, IFNA17, IFNA21, IFNB1, IFNB3, IFNW1, or IFNK protein or variant thereof comprises, consists, or consists essentially of an amino acid sequence that is at least 80, 85, 90, 95, 98, or 100% identical to a sequence selected from Table S5, which is optionally a human IFNA2 variant that comprises or retains a K23 substitution, as defined by the mature IFNA2 sequence, optionally a K23R substitution; and
the IFNAR2 protein or variant thereof comprises, consists, or consists essentially of an amino acid sequence that is at least 80, 85, 90, 95, 98, or 100% identical to a sequence selected from Table S12, which optionally comprises or retains a substitution at M47 and/or E78, as defined by the mature IFNAR sequence, optionally an M47A, M47V, and/or E78A substitution.

13. The activatable proprotein homodimer of claim 7, wherein the type II IFN protein is IFNγ, optionally the IFNG1 subunit and/or the IFNG2 subunit, including cytokine variants thereof, optionally wherein the IFNγ protein or variant thereof comprises, consists, or consists essentially of an amino acid sequence that is at least 80, 85, 90, 95, 98, or 100% identical to a sequence selected from Table S6; and
the IFNGR1 protein or variant thereof comprises, consists, or consists essentially of an amino acid sequence that is at least 80, 85, 90, 95, 98, or 100% identical to a sequence selected from Table S13.

14. The activatable proprotein homodimer of claim 7, wherein the type III IFN protein is IFN-λ, optionally selected from one or more of IFN-λ1, IFN-λ2, IFN-λ3, and IFN-λ4 protein, including cytokine variants thereof.

15. The activatable proprotein homodimer of claim 14, wherein
the IFN-λ protein or variant thereof comprises, consists, or consists essentially of an amino acid sequence that is at least 80, 85, 90, 95, 98, or 100% identical to a sequence selected from Table S7; and
the IL10Rβ or IFN-λR1 protein or variant thereof comprises, consists, or consists essentially of an amino acid sequence that is at least 80, 85, 90, 95, 98, or 100% identical to a sequence selected from Table S14.

16. The activatable proprotein homodimer of any one of claims 1-15, wherein the binding moieties do not bind to the cytokine or the cytokine receptor.

17. The activatable proprotein homodimer of any one of claims 1-16, wherein the binding moieties of the first polypeptide and the second polypeptide bind together, optionally homodimerize, via at least one non-covalent interaction.

18. The activatable proprotein homodimer of any one of claims 1-17, wherein the binding moieties of the first polypeptide and the second polypeptide bind together, optionally homodimerize, via at least one covalent bond.

19. The activatable proprotein homodimer of claim 18, wherein the at least one covalent bond comprises at least one disulfide bond.

20. The activatable proprotein homodimer of any one of claims 1-19, wherein the binding moieties of the first polypeptide and the second polypeptide are selected from Table M1.

21. The activatable proprotein homodimer of any one of claims 1-20, wherein the binding moieties of the first polypeptide and the second polypeptide comprise an antigen binding domain of an immunoglobulin, including antigen binding fragments and variants thereof.

22. The activatable proprotein of any one of claims 1-21, wherein the binding moieties of the first polypeptide and the second polypeptide comprise, consist, or consist essentially of a CH1, CH2, CH3, CH1CH3, CH2CH3, CH1CH2CH3, and/or CL domain of an immunoglobulin, including fragments and variants thereof.

23. The activatable proprotein homodimer of claim 21 or 22, wherein the binding moieties of the first polypeptide and the second polypeptide comprise, in an N- to C-terminal orientation: (1) an antigen binding domain of an immunoglobulin, including antigen binding fragments and variants thereof; and (2) a CH1, CH2, CH3, CH1CH3, CH2CH3, CH1CH2CH3, and/or CL domain of an immunoglobulin, including fragments and variants thereof.

24. The activatable proprotein homodimer of any one of claims 21-23, wherein the antigen binding domain comprises a VH or VL domain of an immunoglobulin, including antigen binding fragments and variants thereof.

25. The activatable proprotein homodimer of any one of claims 1-24, wherein the binding moieties of the first polypeptide and the second polypeptide do not bind to an antigen.

26. The activatable proprotein homodimer of any one of claims 1-25, wherein the binding moieties of the first polypeptide and the second polypeptide comprise a CH2CH3 domain of an immunoglobulin.

27. The activatable proprotein homodimer of any one of claims 21-26, wherein the immunoglobulin is from an immunoglobulin class selected from IgG1, IgG2, IgG3, IgG4, IgA, IgD, IgE, and IgM.

28. The activatable proprotein homodimer of any one of claims 1-27, wherein the binding moieties of the first polypeptide and the second polypeptide comprise a leucine zipper peptide.

29. The activatable proprotein homodimer of any one of claims 1-28, wherein the cleavable linker comprises a protease cleavage site, optionally wherein the cleavable linker is selected from Table L2.

30. The activatable proprotein homodimer of claim 29, wherein the protease cleavage site is cleavable by a protease selected from one or more of a metalloprotease, a serine protease, a cysteine protease, and an aspartic acid protease.

31. The activatable proprotein homodimer of claim 29 or 30, wherein protease cleavage site is cleavable by a protease selected from one or more of MMP1, MMP2, MMP3, MMP4, MMP5, MMP6, MMP7, MMP8, MMP9, MMP10, MMP11, MMP12, MMP13, MMP14, TEV protease, matriptase, uPA, FAP, Legumain, PSA, Kallikrein, Cathepsin A, and Cathepsin B.

32. The activatable proprotein homodimer of any one of claims 1-31, wherein the first linker and/or the second linker are about 1-50 1-40, 1-30, 1-20, 1-10, 1-5, 1-4, 1-3 amino acids in length, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acids in length.

33. The activatable proprotein homodimer of any one of claims 1-32, wherein the first linker is a cleavable linker, and the second linker is a non-cleavable linker.

34. The activatable proprotein homodimer of claim 33, wherein cleavage, optionally protease cleavage, of the first linker exposes the binding site(s) of the cytokine that binds to its wild-type cognate receptor on the surface of the immune cell in vitro or in vivo.

35. The activatable proprotein homodimer of any one of claims 1-34, wherein the first linker is non-cleavable linker, and the second linker is a cleavable linker.

36. The activatable proprotein homodimer of claim 35, wherein cleavage, optionally protease cleavage, of the second linker exposes the binding site(s) of the cytokine that binds to its wild-type cognate receptor on the surface of the immune cell in vitro or in vivo.

37. The activatable proprotein homodimer of any one of claims 1-36, wherein the immune cell is selected from one or more of a T cell, a B cell, a natural killer cell, a monocyte, and a macrophage.

38. The activatable proprotein homodimer of any one of claims 1-37, wherein the first polypeptide and the second polypeptide comprise, consist, or consist essentially of an amino acid sequence that is at least 80, 85, 90, 95, 98, or 100% identical to a sequence selected from Table S15.

39. The activatable proprotein homodimer of any one of claims 1-38, which is substantially in homodimeric form in a physiological solution, or under physiological conditions, optionally in vivo conditions.

40. A recombinant nucleic acid molecule encoding the activatable proprotein homodimer of any one of claims 1-39.

41. A vector comprising the recombinant nucleic acid molecule of claim 40.

42. A host cell comprising the recombinant nucleic acid molecule of claim 40 or the vector of claim 41.

43. A method of producing an activatable proprotein, comprising culturing the host cell of claim 42 under culture conditions suitable for the expression of the activatable proprotein homodimer, and isolating the activatable proprotein from the culture.

44. A pharmaceutical composition, comprising the activatable proprotein homodimer of any one of claims 1-43, and a pharmaceutically acceptable carrier.

45. A method of treating disease in a subject, comprising administering to the subject a therapeutically effective amount of the pharmaceutical composition of claim 44.

46. The method of claim 45, wherein the disease is selected from one or more of a cancer, a viral infection, and an immune disorder.

47. The method of claim 46, wherein the cancer is a primary cancer or a metastatic cancer, and is selected from one or more of melanoma (optionally metastatic melanoma), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer.

48. The method of any one of claims 45-47, wherein following administration, the activatable proprotein homodimer is activated through protease cleavage in a cell or tissue, optionally a cancer cell or cancer tissue, which exposes the binding site(s) of the cytokine that binds its wild-type cognate receptor on the surface of the immune cell in vitro or in vivo, and thereby generates an activated protein.

49. The method of claim 48, wherein:
the activated protein binds via the IL-7 protein to IL-7Rα/λc on the surface of the immune cell;
the activated protein binds via the IL-21 protein to IL-21R/λc on the surface of the immune cell;
the activated protein binds via the type I IFN protein to IFNAR1/IFNAR2 on the surface of the immune cell;
the activated protein binds via the type II IFN protein to IFNGR1/IFNGR2 on the surface of the immune cell; or
the activated protein binds via the type III IFN protein to IL10Rβ/IFN-λR1 on the surface of the immune cell.

50. The method of claim 49, wherein the immune cell is selected from one or more of a T cell, a B cell, a natural killer cell, a monocyte, and a macrophage.

51. The method of any one of claims 45-50, wherein administration and activation of the activatable proprotein increases an immune response in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control, optionally wherein the immune response is an anti-cancer or anti-viral immune response.

52. The method of any one of claims 45-51, wherein administration and activation of the activatable proprotein increases cell-killing in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control, optionally wherein the cell-killing is cancer cell-killing or virally-infected cell-killing.

53. The method of claim 46, wherein the viral infection is selected from one or more of human immunodeficiency virus (HIV), Hepatitis A, Hepatitis B, Hepatitis C, Hepatitis E, Caliciviruses associated diarrhoea, Rotavirus diarrhoea, Haemophilus influenzae B pneumonia and invasive disease, influenza, measles, mumps, rubella, Parainfluenza associated pneumonia, Respiratory syncytial virus (RSV) pneumonia, Severe Acute Respiratory Syndrome (SARS), Human papillomavirus, Herpes simplex type 2 genital ulcers, Dengue Fever, Japanese encephalitis, Tick-borne encephalitis, West-Nile virus associated disease, Yellow Fever, Epstein-Barr virus, Lassa fever, Crimean-Congo haemorrhagic fever, Ebola haemorrhagic fever, Marburg haemorrhagic fever, Rabies, Rift Valley fever, Smallpox, upper and lower respiratory infections, and poliomyelitis, optionally wherein the subject is HIV-positive.

54. The method of claim 46, wherein the immune disorder is selected from one or more of type 1 diabetes, vasculitis, and an immunodeficiency.

55. The method of any one of claims 45-54, wherein the pharmaceutical composition is administered to the subject by parenteral administration.

56. The method of claim 55, wherein the parenteral administration is intravenous administration.

57. Use of a pharmaceutical composition of claim 44 in the preparation of a medicament for treating a disease in a subject.
